SEQUENCE LISTING (11 sequences)

SEQ ID NO: 1 - DNA, 3616 bp, Homo sapiens
  misc_binding (1)..(3616): MAR 1_68 of chromosome 1
gactctagattataccaacctcataaaataagagcatatataaaagcaaatgctcttatcttgcagatccctgaactgaggaggcaagatcagtttggcagttgaagcagctggaatctgcaattcagagaatctaagaaaagacaaccctgaagagagagacccagaaacctagcaggagtttctccaaacattcaaggctgagggataaatgttacatgcacagggtgagcctccagaggcttgtccattagcaactgctacagtttcattatctcagggatcacagattgtgctacctattgcctaccatctgaaaacagttgcttcctatatttcatccagtttaatatttatttaaaccaagaaggttaatctggcaccagctattccgttgtgagtggatgtgaaagtaccaattccattctgttttactattaactatcctttgccttaatatgtatcagtaggtggcttgttgctaggaaatattaaatgaatggcatgtttcataggttgtgtttaaagttgttttttgagttaaatctttctttaataatactttctgatgtcaaaaacacttagaagtcatggtgttgaacatctatatagggttggatctaaaatagcttcttaacctttcctaaccactgtttttgtttgtttgtttttaactaagcatccagtttgggaaattctgaattaggggaatcataaaaggtttcattttagctgggccacataaggaaagtaagatatcaaattgtaaaaatcgttaagaacttctatcccatctgaagtgtgggttaggtgcctcttctctgtgctcccttaacatcctattttatctgtatatatatatattcttccaaatatccatgcatgggaaaaaaaatctgatcataaaaatattttaggctgggagtggtggctcacgcctgtaatcccagcactttgggaggctgaggtgggcggatcatgaggtcaagagatcgagaccatcctgaccaatatggtgaaaccccatctctactaaagatacaaaactattagctggacgtggtggcacgtgcctgtagtcccagctactcgggaggctgaggcaggagaacggcttgaacccaggaggtggaggttgcagtgagctgagatcgcgccactgcactccagcctgggcgacagagcgagactctgtctcaaaaaaaaaatatatatatatatatatatacacatatatatataaaatatatatatatacacacatatatatataaaatatatatatatacacacatatatataaaatatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatataaaatatatatatacacacatatataaaatatatatatacacatatatataaaatatatatatacacatatatataaaatatatatacacacatatatataaaatatatatatacacacatatatataaaatatatatatacacatatatataaaatatatatatacacatatatataaaatatatatatatacacatatatataaaatatatatacacacatatatataaagtatatatatacacacatatatataaaatatatatatacacatatatataaaatatatatatacacatatatataaaatatatatatacacatatatataaaaatatatatatatattttttaaaatattccaattgtctcactttgtggatgagaaaaagaagtagttagaggtcaagtaacttggcctacatcttttctcaagattgtaaactcctagtgagcaataaccacatcttcattttctttgtataaaacaagaaagtttagcatgaaaaaggtactcaattacaaatgtgttggattgaattgaagacccttggaaggggattttgtacctgaggatctctttcttttggccatattgttcaatggacaaaatttagccttcgaaggcaggccgatttgaggttaatactacctttaccacttgatagctatgtgaccttggccatgtggtttcaacagtctgaacctcattttctctgtgtatgtgtggtcctccttacaagtttgtgaaaaatgtgaagtccttagccatgatagcccaatataacaggctaaatgataataggtttatgttcttttcctttatattctcagataagcactgtccaagtttgaggtgttttgaggtctcgcctgatttggattgtttgagtttatgctattctttgaattctttgagctgttctgaagcagtgtatcatgaacaaaaacatccccagttcagtccaaacccctggttacatatcattcttatgccatgttataaccagtttgagagtgttccctctgttattgcatttaagtttcagcctcacacagaaattcagcagccaatttctaagccctaagcataaaatctggggtgggggggggggatggcctgaagagcagcattatgaatagcaccattataattaatgatctctcaggaagatttacaatcacaggtagcagataaaacaaatagtactgcttctgcacttcccctccttttattcgctatgaaattttatgggaaatcagtccagtgaaaaatgtaagctcttaatctttcccagaaatcctacctcatttgatgaatactttgagggaatgaattagagcatttttttcttttatagtctacttcgcatttacgaagtgaggacggtagcttaggctgcctggccaactgatgagaaggtcagaggcatttttagagacctctgttgtctttcattcatgttcattttccacaaggcaagtaatttccaacaaatcagtgtcttcattagtaataagattattaacaacaataatagtcatagtaactattcagtgagagtccattatatatcaggcattctacaaggtactttatatacatctgagtaaacctcacacaattctacagggaggtatttctatccccatttaacaaataaggaaacgaagtccaagtaaattaacttgcccaaggtcacacagatagtacctggcagaacaggaatttaaacctaaatttgtccaactccaaaagcagccttctatttgttataaatgctgcctctcattatcacatattttattattaacaacaacaaacataccaattagcttaagatacaatacaaccagataatcatgatgacaacagtaattgttatactattataataaaatagatgttttgtatgttactataatcttgaatttgaatagaaatttgcatttctgaaagcatgttcctgtcatctaatatgattctgtatctattaaaatagtactacatctagag

SEQ ID NO: 2 - DNA, 4624 bp, Homo sapiens
  misc_binding (1)..(4624): MAR 1_6 of chromosome 1
ggatcttaaatctattttatttatttatttttcatgtggccaataccctccacccccttcttctgtctctttcaacttattgtggttaccttgaggctacctgagacagtaggcttgggtggggaagtatgcattctaagtgtaaagtttgatgagctttgacaaatgtcaacccatgtaccagaacattttcatcacccataaaatctcccttgtgtcacttgccagtcagtgtctattctagtatccaactcctggctccaagaaaccattgaactgttttctgtcactataaattagatttgtcttttctagagtttcatgtaaatggaatcatacactaagtactctttgtgcctggcttctgctcagcataatgtttttgagaatcattcatgctgctgcatgttttcagtagttcatttttttaaataggtgaattgtaactcattctgtgaatataccatattctgtcttccatttatctgttagtggatctttaggtcgtttctagttttgggctattgcaaataaagctgctgtaaatattaatgcacaagttttccatgttcatatgtttcatttcacttaggaaaatacctaagagaggaattgcacatattaaaaaaattttaaaaactactaagctgttctccaaaatggttgtacaatttttattcccaagagcaatatgagtgtttaattgctccacattctcaccaacacttggtgcttgttagttttattttcattgttttcattgttatgtctgtgaggcagcattgatgtgcatgtctctgagtgtcatcttagcggtgatgctgagcatcagttcacgtccttataggccgtttgtatatctgctttgtgaaatgtctgttcaaatcttttgcctattttaaattgagttgtgttcgtcttcttaggattaagtaatgagttaaaaatatttctgatacaaatctttcattatatatttctaatgctttctcatctatagtttattttctcatattctttaactgtatcttttgaagagcaaattttacttttgattatgcccaatttatcaagtttttatatggctcttttgattatgcccataatcacattagactttgcctaacccaagtttgcagagattttttcttttatgcttttatctagaaattttgtagttttaggttttaaaaaagtttaatttatttatttgagacagggtattgctctttacatatactggagtgcagtgatgcaatcatggctcactgcagcctcaacctcttgggctcaagcggttctcccatctcagagtcctgagtagctggccaggtgcatgccagcttcaatgtgtttttcatttgcatttccctgataattattgacgttgagcatttttttcatatatcagttagctatttgtacgtcttcttttgagaaacatctattcgggtcttttgcccattttaaagtcagatggtttgtttgtcagctattgagttgtttgagttccttgtatattctggatattaccatcttgtcagatgcacagtttgcaaattttttttttctattttgtaggttgtctctttctctgttgtttcctccggtatgcagaagttttttagtgtgatgtaatttcatttgtctgtttttgcttttgttgcctgtactttcttattcttatccaaaaaatctttatctagatcaatgtcacgaagagtttctcctctgttttcttcgagtagttttttataattttgggtatacatttaagtctttaatctatttggaattgatttttgcatatggtgagagatcagagtctaatttcatacttttggatgtggaaagctagttttttcagcaccatttattgaagagactgtctcttctccaatgtgtgttctttgtgccttcgtcaaaaatcagttggctgtgcgtggatttatttctgtgttctctattttgttccattggtctagttttagccttaaatttaggtctgcaatttttttttttttgtatatggtgtgaagtaagagtcaaagttcattatttttcatatggatatgtaattactccagtaccatcatttagtttgaatggactgtcctttctccatggaattacatgggcatcttttgtctgaaaccaattatgtatgtttacgtatgtgtatgtttatgcatatgttataggtttaatatatattaatatatataatatataatatataaatattaatatgtattatataatatatattaatatattatattatattactatataaataatattaatatattatattaaaatattaataaatatatcatattaaatattatattaattaaatattaataaatatattatattaatatatttatatattaaacctataacatatgcatatacttatttatatataacatgcatgtacttatttatatatacaatatatatttatatattatataatatattatatgtatttatatattatatatcatatattatatgtatttatatattatatatcatataatatatatatttatattatatatattatatgatatataatattatataatgtattaatatatattaaacctatatttataattctggactcactattttgtttcattggtgtctgtgtgtatctaaccctatgccaataatgtactatcttaattaccatagctttatagtaagctttgaaatcagatagtgtattttttatcattgttttttaaaataatagtttatctttttatttgaatttgtaatcagctagtcagtttctgcaaaaagcttactgggattttgcttggaattatgttacatctgtagcatgtactatccaatattctagcctttatccacatgtggctattaaggtttaaattaattaaattaaaatttaattaattaaaattaaaacttaataattggttcctcattcacactaccatatgtcaagtgttcaatagccacatatggtcaatgtcttggaaaagtcaatacagtacatttccattattgcagtaagttctgtcaaacagcactatcgtagaccgattaggagagaactgacttaacagtattggatgctccagtcaatgaacatctttttttttttcatttatttcagtagtctctgcagtatattatagatttcagtttacatattttgcatatattttattaaatgtataacggtagaagtactattattggatgatgtgttctatagatgtattttaggtcaagtttgttgatagtgttgtttaaatctcgtatacctcttgatttttttatttacttgttctttgaattactgagacaggaatgttatatccttaactatatttgtgaatttattcacttcttccttcagttctgttaacttttgcttaggtgctttttaaaaatgaaactttcaatctctgccttttaattgtagcatttagaccatttacattcaatgtaattatcaatatcagtttatttaagtctgaagttgtgcaatttttcctctacctatattataaatctttctatatacaaaacacatgctatgttttctgcatatgttttaaatgacacccggaaagcattgacactatttttgctttaggttatctttcaaagatgttaaaaatgagaaagaaatattctgcatttatccatacacttattatttgcaaaggtttttttaaatacctttgtgtagatttcagttaccaacttgtatttccttcagcttgaagaacttacaatttcttgtaggacaggtctctgacaacaaattatctcagcttttctttgtctaaaaaagttattgcctttatttttaaaatatattttcactggatattgaattttaggtgataatctttttttttttgttagcactttaaatatgtcttctaatgtcctcttgctttcatagtttctgatgagaagtctactgttattagtatctctttgtgtgtgtctctcttttttccctctctgctattatggctatttttttttttttttttttttttggtcactggtgtcagcaatttaattatggtgtgccttggtatgttttgtgtgtgtgtgtgtgtgtgtgtgtgtgtgtgtgtgtgtgtagctgatgttctttgagctttagaatctgtgagtttgtagttttcatcaattattttttcttttcattccttttatttactcatgttcgtgttttattttatatttttaagaattttgtgcgtatttgtaataactgtttaaatgtcatttgtgaattccattgcttctaggtaggattctattgacagatattttttccctgacgagaggtcatactttccttattcttcatgtatctagtggtttttggttgaatactggatattttgaattttatgggagtgctgaattctacaatattccttaaaaatgtgttggattttgttttagcagatagctatcttacttgaagatcaatttcatattttttgatgttcattttttcatttattaaagaataggtccatggtagagtttactgatatcaacctttctggtgtctctaataaatgcaacatattcaataagatcc

SEQ ID NO: 3 - DNA, 3354 bp, Homo sapiens
  misc_binding (1)..(3354): MAR X_S29 of chromosome X
gatccctttataaaaccacaatataatggagtgctataatttcaaacagtgtttggtctgctggcagagtggtcattctaacagcagtcacagtagagtagaaataagactgcagtatatctaaggcaaaaagctgaggtttcaggagcttgaaggtaaagaggaagaaagaaatgggaatgggaattggaaagacaaatatcgttaagagaaaattgcttttaggagaggggaaagaatctatgtgtacttaagactatggaatcaatcccatttaagctgggaaactagtttcatatataactaataaattttatttacagaatatctatttacctgatctaggcttcaagccaaagggactgtgtgaaaaaccatcagttctgtcatattcctaaaaaaaaattaaaaagttaaaaataaataaataataaaacttcttttctttcaaaataatcaaggtgcttattcacatccattccaatttggggaaatacttattttcctatgattagcgaagagaaaagtaacttgcatttcaattcaagttgatacatgtcacttttaagaggtcaactaatatttgctagttgagctaaccatataggctttaaatactttcatagtagaaagaaaatgaaaatcattagtgaactgtataaaatagatcatactttttgaaagaatcagactgaagtttccgaaaaaaagaagtaagcttcaatgaaaaggtaagtgaatttagcatttactcagcatctactatggacttaacacctaacagtagataatctgaaggcaaacatatttgtatagggactgcagaatgatagatgataaatatcatctcttctatttgaatgaatattttttcaaatctttcacacacagtggtttgctatggaaagatttgtagtacattaaacaaatctgaagatggagttagaaagcttaggctatgttttgagcacaacatataatttctctgtgattgtttcttcatctttcaaatgaggttactgtgaagattaaatgagataactaaatgatgataaaataatgtaatcttagcagcaccttatttaatctgtgcaacaactctgtgaagtgagtagggctcagcttcagtcacttctctgccatttattaactaagatagtttggaaagttacccatctcttcagctgtaaaatgatgaggatcatacctattttatggggctgcttttaggtacaaatatacaggcaagcactttgttaatactaaagcattacaccaattagttttactcttttccattcacacatgaaattaatgtaatcagaattctgtagattacctaaatcttctgttaacacgtgatatgcagttcaggttaaatgtcagttgagttaccaaagcacatacatactcaccaccctatccaaatctacaagcctcccagtttgtcttcactattttggttaaattaatatgaattcctagatgaaaatttcactgatccaaatgaaataaaaaatatattacaaaactcacacctgtaatctcaacattttgggaggccaaggcaggtagatcacttgaggccaggagttcaagaccagcctgatcaacatggtgaaaccctgtctctactaaaaatacaaaaattagccaggtgtggtggcatgtgcctgtagtcctacctactcgggaggctgaggcacaagaatcgcttgaatgtgggaggtggaggttgcagtgacctgagatcgtgccactgcactccagcctaggcaacagagtgagatcatgtgtcatatatatatatatatatatatatatatatatatatatatacacacacacacacatatatatatacacatatatatacgtatatatatatatgtatatatatacatatatatacatatatatatatacgtatatatatacgtatatatatatcaatgtaaattatttgggaaatttggtatgaatagtcttccctgtgaacacagatcataaaatcatatatcaagcagacaaataagtagtagtcacttatatgcttatacttgtaacttaaagtaaaagaattacaaaagcatatgacaaagactaattttaagatatcctaatttaaattgttttctaaaagtgtgtataccattttacctatcatatgaataatttagaaacatgtttataaaattaatgtccaaatccattcaaaagttttgtaatgcagatcacccacaacaacaaagaatcctagcctattaaaaaagcaacaccacctacatataatgaaatattagcagcatctatgtaaccaaagttacacagtgaatttgggccatccaacactttgagcaaagtgttgaattcatcaaatgaatgtgtaatcatttacttactaatgccaatacactttaaggtaatcttaagtagaagagatagagtttagaattttttaaatttatctcttgttgtaaagcaatagacttgaataaataaattagaagaatcagtcattcaagccaccagagtatttgatcgagatttcacaaactctaactttctgatacccattctcccaaaaacgtgtaacctcctgtcgataggaacaacccactgcagggatgtttctcgtggaaaaaggaaatttcttttgcattggtttcagacctaactggttacaagaaaaaccaaaggccattgcacaatgctgaagtacttttttcaaatttaaaatttgaaagttgttcttaaaatctatcatttattttaaaatacggatgaatgagaaagcatagatttgataaagtgaattcttttctgcaatctacagacacttccaaaaatcactacagacactacagacactacagaaaatcataaataaacaagtgctagtatcaatatttttaccaaaaaatggcattcttagaattttttataggctagaaggtttgtacaaactaatctgccacggattttaaaatatgagtgaataaattatattgcaaaaaaaatcaggttacagagaactggcaaggaagactcttatgtaaaacacagaaaacatacaaaacgtatttttaagacaaataaaaacagaacttgtacctcagatgatactggagattgtgttgacatattagcattatcactgtcttgctaaaacataaaaataaaaagatggaagatgaaattacaatacaaatgatgatttaaacatataaaaggaaaataaaaattgttctgaccaactactaaaggaagacctactaaagatatgccatccagcacattgccactctacatgtggtctgtaaaccagcagcatagggatcctctagctagagt

SEQ ID NO: 4 - DNA, 1515 bp, Homo sapiens
  SRP54 (1)..(1515): c DNA of subunit 54 of the SRP particle
atggttctagcagaccttggaagaaaaataacatcagcattacgctcgttgagcaatgccaccattatcaatgaagaggtattgaatgctatgctaaaagaagtctgtaccgctttgttggaagcagatgttaatattaaactagtgaagcaactaagagaaaatgttaagtctgctattgatcttgaagagatggcatctggtcttaacaaaagaaaaatgattcagcatgctgtatttaaagaacttgtgaagcttgtagaccctggagttaaggcatggacacccactaaaggaaaacaaaatgtgattatgtttgttggattgcaagggagtggtaaaacaacaacatgttcaaagctagcatattattaccagaggaaaggttggaagacctgtttaatatgtgcagacacattcagagcaggggcttttgaccaactaaaacagaatgctaccaaagcaagaattccattttatggaagctatacagaaatggatcctgtcatcattgcttctgaaggagtagagaaatttaaaaatgaaaattttgaaattattattgttgatacaagtggccgccacaaacaagaagactctttgtttgaagaaatgcttcaagttgctaatgctatacaacctgataacattgtttatgtgatggatgcctccattgggcaggcttgtgaagcccaggctaaggcttttaaagataaagtagatgtagcctcagtaatagtgacaaaacttgatggccatgcaaaaggaggtggtgcactcagtgcagtcgctgccacaaaaagtccgattattttcattggtacaggggaacatatagatgactttgaacctttcaaaacacagccttttattagcaaacttcttggtatgggcgacattgaaggactgatagataaagtcaacgagttgaagttggatgacaatgaagcacttatagagaagttgaaacatggtcagtttacgttgcgagacatgtatgagcaatttcaaaatatcatgaaaatgggccccttcagtcagatcttggggatgatccctggttttgggacagattttatgagcaaaggaaatgaacaggagtcaatggcaaggctaaagaaattaatgacaataatggatagtatgaatgatcaagaactagacagtacggatggtgccaaagtttttagtaaacaaccaggaagaatccaaagagtagcaagaggatcgggtgtatcaacaagagatgttcaagaacttttgacacaatataccaagtttgcacagatggtaaaaaagatgggaggtatcaaaggacttttcaaaggtggcgacatgtctaagaatgtgagccagtcacagatggcaaaattgaaccaacaaatggccaaaatgatggatcctagggttcttcatcacatgggtggtatggcaggacttcagtcaatgatgaggcagtttcaacagggtgctgctggcaacatgaaaggcatgatgggattcaataatatgtaa

SEQ ID NO: 5 - DNA, 249 bp, Homo sapiens
  SRP9 (1)..(249): cDNA of subunit 9 of the SRP particle
atgccgcagtaccagacctgggaggagttcagccgcgctgccgagaagctttacctcgctgaccctatgaaggcacgtgtggttctcaaatataggcattctgatgggaacttgtgtgttaaagtaacagatgatttagttagacagtgtcttgctctattgctcaggctgcagtgcagtggcatgatcatagctcactgcatcctcgacctcctgggctcaagcggtcctcttgcttcagcctcctga

SEQ ID NO: 6 - DNA, 411 bp, Homo sapiens
  SRP14 (1)..(411): cDNA of subunit 14 of the SRP particle
atggtgttgttggagagcgagcagttcctgacggagctgaccagacttttccagaagtgccggacgtcgggcagcgtctatatcaccttgaagaagtatgacggtcgaaccaaacccattccaaagaaaggtactgtggagggctttgagcccgcagacaacaagtgtctgttaagagctaccgatgggaagaagaagatcagcactgtggtgagctccaaggaagtgaataagtttcagatggcttattcaaacctccttagagctaacatggatgggctgaagaagagagacaaaaagaacaaaactaagaagaccaaagcagcagcagcagcagcagcagcagcacctgccgcagcagcaacagcagcaacaacagcagcaacaacagcagcaacagcagcacagtaa

SEQ ID NO: 7 - DNA, 1917 bp, Homo sapiens
  SR_alpha (1)..(1917): cDNA of subunit alpha of the SRP rececptor
atgctcgacttcttcaccattttctccaagggcgggcttgtgctctggtgcttccagggcgttagcgactcatgcaccggacccgttaacgcgttgattcgttccgtgctgctgcaggaacggggaggtaacaactccttcacccatgaggcactcacactcaagtataaactggacaaccagtttgagctggtgtttgtggttggttttcagaagatcctgacactgacatatgtagacaaattgatagatgacgtgcatcggctgtttcgggacaagtaccgcacagagatccaacagcaaagtgctttaagtttattaaatggcacttttgatttccaaaatgacttcctgcggctccttcgtgaagcagaggagagcagtaagatccgtgctcccactaccatgaagaaatttgaagattctgaaaaggccaagaaacctgtgaggtccatgattgagacacggggggaaaagcccaaggaaaaagcaaagaatagcaaaaaaaagggggccaagaaggaaggttctgatggtcctttggctaccagcaaaccagtccctgcagaaaagtcaggtcttccagtgggtcctgagaacggagtagaactttccaaagaggagctgatccgcaggaagcgcgaggagttcattcagaagcatgggaggggtatggagaagtccaacaagtccacgaagtcagatgctccaaaggagaagggcaaaaaagcaccccgggtgtgggaactgggtggctgtgctaacaaagaagtgttggattacagtactcccaccaccaatggaacccctgaggctgccttgtctgaggacatcaacctgattcgagggactgggtctggggggcagcttcaggatctggactgcagcagctctgatgacgaaggggctgctcaaaactctaccaaacctagtgcgaccaagggaacactgggtggcatgtttggtatgctgaagggccttgtgggttcaaagagcttgagtcgtgaagacatggaatctgtgctggacaagatgcgtgatcatctcattgctaagaacgtggctgcagacattgccgtccagctctgtgaatctgttgccaacaagttggaagggaaggtgatggggacgttcagcacggtgacttccacagtaaagcaagccctacaggagtccctggtgcagattctgcagccacagcgtcgtgtagacatgctccgggacatcatggatgcccagcgtcgccagcgcccttatgtcgtcaccttctgcggcgttaatggagtggggaaatctactaatcttgccaagatttccttctggttgttagagaatggcttcagtgtcctcattgctgcctgtgatacatttcgtgctggggccgtggagcagctgcgtacacacacccggcgtttgagtgccctacaccctccagagaagcatggtggccgcaccatggtgcagttgtttgaaaagggctatggcaaggatgctgctggcattgccatggaagccattgcttttgcacgtaaccaaggctttgacgtggtgctggtggacacggcaggccgcatgcaagacaatgcccctctgatgactgccctggccaaactcattactgtcaatacacctgatttggtgctgtttgtaggagaagccttagtaggcaatgaagccgtggaccagctggtcaagttcaacagagccttggctgaccattctatggctcagacacctcggctcattgatggcattgttcttaccaaatttgataccattgatgacaaggtgggagctgctatttctatgacgtacatcacaagcaaacccatcgtctttgtgggcaccggccagacctactgtgacctacgcagcctcaatgccaaggctgtggtggctgccctcatgaaggcttaa

SEQ ID NO: 8 - DNA, 816 bp, Homo sapiens
  SRbeta (1)..(816): cDNA of subunit beta of the SRP receptor
atggcttccgcggactcgcgccgggtggcagatggcggcggtgccgggggcaccttccagccctacctagacaccttgcggcaggagctgcagcagacggacccaacgctgttgtcagtagtggtggcggttcttgcggtgctgctgacgctagtcttctggaagttaatccggagcagaaggagcagtcagagagctgttcttcttgttggcctttgtgattccgggaaaacgttgctctttgtcaggttgttaacaggcctttatagagacactcagacgtccattactgacagctgtgctgtatacagagtcaacaataacaggggcaatagtctgaccttgattgaccttcccggccatgagagtttgaggcttcagttcttagagcggtttaagtcttcagccagggctattgtgtttgttgtggatagtgcagcattccagcgagaggtgaaagatgtggctgagtttctgtatcaagtcctcattgacagtatgggtctgaagaatacaccatcattcttaatagcctgcaataagcaagatattgcaatggcaaaatcagcaaagttaattcaacagcagctggagaaagaactcaacaccttacgagttacccgttctgctgcccccagcacactggacagttccagcactgcccctgctcagctggggaagaaaggcaaagagtttgaattctcacagttgcccctcaaagtggagttcctggagtgcagtgccaagggtggaagaggggacgtgggctctgctgacatccaggacttggagaaatggctggctaaaattgcctga

SEQ ID NO: 9 - DNA, 1431 bp, Homo sapiens
  Sec61A1_(alpha) (1)..(1431): cDNA of subunit alpha (Sec61 alpha) of the translocon
atggcaatcaaatttctggaagtcatcaagcccttctgtgtcatcctgccggaaattcagaagccagagaggaagattcagtttaaggagaaagtgctgtggaccgctatcaccctctttatcttcttagtgtgctgccagattcccctgtttgggatcatgtcttcagattcagctgaccctttctattggatgagagtgattctagcctctaacagaggcacattgatggagctagggatctctcctattgtcacgtctggccttataatgcaactcttggctggcgccaagataattgaagttggtgacaccccaaaagaccgagctctcttcaacggagcccaaaagttatttggcatgatcattactatcggccagtctatcgtgtatgtgatgaccgggatgtatggggacccttctgaaatgggtgctggaatttgcctgctaatcaccattcagctctttgttgctggcttaattgtcctacttttggatgaactcctgcaaaaaggatatggccttggctctggtatttctctcttcattgcaactaacatctgtgaaaccatcgtatggaaggcattcagccccactactgtcaacactggccgaggaatggaatttgaaggtgctatcatcgcacttttccatctgctggccacacgcacagacaaggtccgagcccttcgggaggcgttctaccgccagaatcttcccaacctcatgaatctcatcgccaccatctttgtctttgcagtggtcatctatttccagggcttccgagtggacctgccaatcaagtcggcccgctaccgtggccagtacaacacctatcccatcaagctcttctatacgtccaacatccccatcatcctgcagtctgccctggtgtccaacctttatgtcatctcccaaatgctctcagctcgcttcagtggcaacttgctggtcagcctgctgggcacctggtcggacacgtcttctgggggcccagcacgtgcttatccagttggtggcctttgctattacctgtcccctccagaatcttttggctccgtgttagaagacccggtccatgcagttgtatacatagtgttcatgctgggctcctgtgcattcttctccaaaacgtggattgaggtctcaggttcctctgccaaagatgttgcaaagcagctgaaggagcagcagatggtgatgagaggccaccgagagacctccatggtccatgaactcaaccggtacatccccacagccgcggcctttggtgggctgtgcatcggggccctctcggtcctggctgacttcctaggcgccattgggtctggaaccgggatcctgctcgcagtcacaatcatctaccagtactttgagatcttcgttaaggagcaaagcgaggttggcagcatgggggccctgctcttctga

SEQ ID NO: 10 - DNA, 291 bp, Homo sapiens
  Sec61B_(beta) (1)..(291): cDNA of subunit beta (Sec61 beta) of the translocon
atgcctggtccgacccccagtggcactaacgtgggatcctcagggcgctctcccagcaaagcagtggccgcccgggcggcgggatccactgtccggcagaggaaaaatgccagctgtgggacaaggagtgcaggccgcacaacctcggcaggcaccggggggatgtggcgattctacacagaagattcacctgggctcaaagttggccctgttccagtattggttatgagtcttctgttcatcgcttctgtatttatgttgcacatttggggcaagtacactcgttcgtag

SEQ ID NO: 11 - DNA, 207 bp, Homo sapiens
  Human_Sec61G_(gamma) (1)..(207): cDNA of subunit gamma (Sec61 gamma) of the translocon
atggatcaggtaatgcagtttgttgagccaagtcggcagtttgtaaaggactccattcggctggttaaaagatgcactaaacctgatagaaaagaattccagaagattgccatggcaacagcaataggatttgctataatgggattcattggcttctttgtgaaattgatccatattcctattaataacatcattgttggtggctga

(19) United States
(12) Patent Application Publication
(10) Pub. No.: US 2012/0231449 A1
(43) Pub. Date: Sep. 13, 2012

(54) PRODUCTS AND METHODS FOR ENHANCED TRANSGENE EXPRESSION AND PROCESSING

(75) Inventors: Nicolas Mermod, Plan-les-Ouates (CH); Pierre-Alain Girod, Plan-les-Ouates (CH); Mélanie Grandjean, Plan-les-Ouates (CH); Valérie Le Fourn, Plan-les-Ouates (CH); David Calabrese, Plan-les-Ouates (CH); Alexandre Regamey, Plan-les-Ouates (CH)

(73) Assignee: SELEXIS S.A., Plan-les-Ouates (CH)

(21) Appl. No.: 13/496,817

(22) PCT Filed: Sep. 20, 2010
(86) PCT No.: PCT/IB10/02337

§ 371 Date: Jun. 4, 2012

Related U.S. Application Data
(60) Provisional application No. 61/243,950, filed on Sep. 18, 2009.

(51) Int. Cl. C12N 15/85; C12Q 1/68; C12N 5/10

(52) U.S. Cl. 435/6.1; 435/455; 435/325; 435/326; 435/358

(57) ABSTRACT

Disclosed are methods and eukaryotic host cells for transgene expression. The cells may be treated and/or modified to increase homologous recombination (HR), decrease non homologous end joining (NHEJ) and/or to enhance a HR/NHEJ ratio in said cell. Such cells can be transfected with vectors comprising the transgene, which advantageously integrates into the genome of the cell to form a concatemeric structure which may comprise more than 200 transgene copies. Certain expression enhancing elements such as MARs are advantageously provided to further enhance and/or facilitate transgene expression. Disclosed is also a recombinant eukaryotic host cell, in particular a non-primate host cell, comprising a transgenic sequence encoding a protein and/or a RNA, in particular a primate protein and/or RNA, involved in translocation across the ER membrane and/or secretion across the cytoplasmic membrane.

### CROSS-REFERENCE TO RELATED APPLICATIONS

This application claims the benefit of U.S. provisional application No. 61/243,950, filed Sep. 18, 2009, which is incorporated herein by reference in its entirety.

### FIELD OF THE INVENTION

The invention is directed at methods and eukaryotic host cells for transgene expression. Transgene expression is boosted by favoring homogenous recombination (HR) over non homologous end joining (NHEJ). The invention is also directed at providing, in an non-primate eukaryotic host cell, proteins involved in primate, in particular human, pathways that mediate or influence translocation across the ER membrane and/or secretion across the cytoplasmic membrane.

### BACKGROUND OF THE INVENTION

The biotechnological production of therapeutical proteins as well as gene and cell therapy depends on the successful expression of transgenes introduced into an eukaryotic cell. Successful transgene expression often requires integration of the transgene into the host chromosome and is limited, among others, by the number of transgene copies integrated and by epigenetic effects that can cause low or unstable transcription and/or high clonal variability. Failing or reduced transport of the transgene expression product out of the cell also often limits production of therapeutical proteins as well as gene and cell therapy.

The publications and other materials, including patents and accession numbers, used herein to illustrate the invention and, in particular, to provide additional details respecting the practice are incorporated herein by reference in their entirety. For convenience, the publications are referenced in the following text by author and date and are listed alphabetically by author in the appended bibliography.

The fact that the DNA of eukaryotes is highly compacted into chromatin allows the entire eukaryotic genome to fit within a nucleus which is a few micrometers diameter. However, this fact entails that gene expression is controlled via the local and temporary condensation and de-condensation of the chromatin, which involves a highly regulated and sophisticated cell machinery. In addition, transgene integration into the host chromosome is, in most cases, a random event resulting in a random integration locus and a varying copy number. The generally observed high degree of variability among independent transformants in stable transgene expression is thought to depend on the number of transgene copies that integrate within the host genome and on the chromatin environment at the site of transgene integration (Kalos and Fournier, 1995; Recillas-Targa et al., 2002). The expression of a transgene integrated into a random locus may be influenced by the arbitrary presence of regulatory elements at the integration locus as well as by the chromatin structure of chromosomal domains adjacent to the integration locus. For instance, a phenomenon called position effect variation can induce silencing of an active gene with time, because of its proximity to repressive heterochromatin (Robertson et al., 1995; Henikoff, 1996; Wakimoto, 1998).

Numerous methods, such as calcium-phosphate DNA co-precipitation, the polyethylenimine method, electroporation and polycationic lipids have been developed to facilitate gene transfer with variable transfection efficiencies. One way to augment the copy number of the transgene and thus increasing transgene expression, is gene amplification (Kaufman, 2000). An alternative is to optimize the expression vector by the insertion of synthetic or natural regulatory sequences.

To increase and stabilize transgene expression in mammalian cells, epigenetic regulators are being increasingly used to protect transgenes from negative position effects (Bell and Felsenfeld, 1999) and include boundary or insulator elements, locus control regions (LCRs), stabilizing and antirepressor (STAR) elements, ubiquitously acting chromatin opening (UCOE) elements and the aforementioned matrix attachment regions (MARs). All of these epigenetic regulators have been used for recombinant protein production in mammalian cell lines (Zahn-Zabal et al., 2001; Kim et al., 2004) and for gene therapies (Agarwal et al., 1998; Allen et al., 1996; Castilla et al., 1998).

As mentioned above, failing or reduced transport of the transgene expression product out of the cell also often limits production of therapeutical proteins as well as gene and cell therapy. The transgene expression product often encounters different bottlenecks: The cell that is only equipped with the machinery to process and transport its innate proteins can get readily overburdened by the transport of certain types of transgene expression products, especially when they are produced at abnormally high levels as often desired, letting the product aggregate within the cell and/or, e.g., preventing proper folding of a functional protein product.

Different approaches have been pursued to overcome transportation and processing bottlenecks. For example, CHO cells with improved secretion properties were engineered by the expression of the SM proteins Munc18c or Sly1, which act as regulators of membranous vesicles trafficking and hence secreted protein exocytosis (U.S. Patent Publication 20090247609). The X-box-binding protein 1 (Xbp1), a transcription factor that regulates secretory cell differentiation and ER maintenance and expansion, or various protein disulfide isomerases (PDI), have also been used to decrease ER stress and increase protein secretion (Mohan et al. 2007). Other attempts to increase protein secretion included the expression of the chaperones ERp57, calnexin, calreticulin and BiP1 in CHO cells (Chung et al., 2004). Finally, expression of a cold shock-induced protein, the cold-inducible RNA-binding protein (CIRP), was shown to increase the yield of recombinant γ-interferon. Attempts were also made to overexpress proteins of the secretory complexes. However, for instance, Lakkaraju et al. (2008) reported that exogenous SRP14 expression in WT human cells (e.g. in cells that were not engineered to express low SRP14 levels) did not improve secretion efficiency of the secreted alkaline phosphatase protein.

Thus, there is a need for efficient, more reliable transgene expression, e.g., recombinant protein production and for gene therapy. There is also a need to successfully transport the transgene expression product outside the cell.

This and other needs in the art are addressed by certain embodiments of the present invention.

### SUMMARY OF THE INVENTION

The present invention is directed at a method for transgene expression comprising (a) providing an eukaryotic, preferably a mammalian, host cell, wherein said host cell has been modified or treated to increase homologous recombination (HR), decrease non homologous end joining (NHEJ) and/or to enhanced HR/NHEJ ratio in said cell, and (b) transfecting said cell, with at least one vector comprising said transgene, and optionally, with a matrix attachment region (MAR) element, wherein said MAR element is provided to said transgene in cis or trans.

The transfection in (b) may be a subsequent transfection, including just a single subsequent transfection, and may be preceded by an initial transfection, including just a single initial transfection, with nucleic acid such as a vector or nucleic acid fragments. The cell cycle of a cell population of said cell may be synchronized, e.g., by subjecting the cell population to a chemical or temperature treatment. The initial and subsequent transfection may take place at a time when a majority of the cells of the population are at the G1 phase of the cell cycle. More than 30%, more than 31%, 32%, 33%, 34%, 35%, 36%, 36%, 38%, 39%, 40%, 41%, 42%, 43%, 44% or 45% of the cells of the cell population may be in the G1 phase. Preferably, prior to the initial transfection an HR enzyme, an HR activator and/or a NHEJ suppressor may be administered. The cell may also be a recombinant eukaryotic host cell and may comprise a transgenic sequence encoding an HR enzyme, an HR activator and/or a NHEJ suppressor. The cell may also be mutated in a NHEJ or a HR gene. Alternatively or additionally, the genome said cell may mutated to inactivate NHEJ, to increases expression or activity of at least one HR enzyme, at least one HR activator and/or at least one NHEJ suppressor.

The nucleic acid of said initial transfection is, in certain embodiments, a vector comprising a transgene. The vector of the initial transfection and at least one vector of said at least one subsequent transfection may form concatemeric structures prior and/or after integration into the genome of the cell. The concatemeric structures may comprise at least 200, 300, 400, 500 or 600 copies of said transgene. The HR/NHEJ ratio of the cell may be up to 2, 3, 4, 5, 6, 7, 8, 9, 10, 15, 20, 25, 30 times higher than a ratio found in the cell not comprising said transgenic sequence and not being mutated, respectively. The NHEJ activity of the cell may equal about 0.

The integrated copy number of said transgene integrated into the genome of said cell following said at least one subsequent transfection may be more than twice that of a reference value representing the integrated copy number obtained by directly transfection the cell with the vector of (b).

The nucleic acid of the initial transfection may be a vector comprising a MAR element and said transgene. Following the initial transfection, e.g., a single initial transfection, the expression of said transgene may reach an initial level and the expression of the transgene following the subsequent transfection, e.g., a single subsequent transfection, may reach a subsequent level that is more than additive, preferably more than twice, three or four times that of said initial level. Alternatively or additionally, after the initial transfection, the transgene copy number integrated into the genome of the cell may equal (n) and following the at least one subsequent transfection, the transgene copy number integrated into the genome may be more than 2(n), 3(n) or 4(n). The transgene may be integrated into the genome of said cell as a concatemeric structure at a single locus.

The MAR element in (b) may ameliorate expression, substantially or fully prevent inhibitory effects from co-integration of multiple copies of the vector comprising the transgene.

More than 50%, 60%, 70%, 80% of the vectors of the at least one subsequent transfection may be transported into the nucleus.

After the initial transfection an initial level of transgene expression product and an initial transgene copy number may be reached. Following said at least one subsequent transfection, the level of transgene expression product may increase to a subsequent level and the initial transgene copy number may increase to a subsequent transgene copy number, wherein the increase between the first and second level of transgene expression product may exceed the increase between the initial transgene copy number and the subsequent transgene copy number by 20%, 30%, 40%, 50% or 60%.

The vector sequence of said vector of the at least one first transfection may have 100% or at least 95%, 90%, 85% or 80% sequence identity with the vector sequence of at least the vector of a first of said subsequent transfection(s). The vector of the initial transfection may comprise a MAR element and said MAR element may have 100% or at least 95%, 90%, 85% or 80% sequence identity with the MAR element of at least the vector of a first of said subsequent transfection(s). The vector of the initial transfection may comprise a transgene and the transgene may have 100% or at least 95%, 90%, 85% or 80% sequence identity with the transgene of at least the vector of a first of said subsequent transfection(s). The MAR element may be provided in cis as part of the vector in (b). In certain embodiments, the transgene is flanked by at least two MAR elements. The MAR element may be located upstream of a promoter/enhancer sequence of said transgene.

The MAR sequence may have at least 90% sequence identity with: SEQ ID NOs: 1-3 or is a variant thereof.

The invention is also directed at a recombinant eukaryotic, preferably mammalian, host cell, comprising
(a) a transgenic sequence expressing a NHEJ suppressor,
(b) a transgenic sequence expressing one or more HR enzyme or HR activator,
(c) a mutation inactivating or downregulating a NHEJ gene, and/or
(d) a mutation enhancing expression or activity of an HR enzyme, an HR activator or a NHEJ suppressor,
wherein the recombinant eukaryotic host cell has an HR/NHEJ ratio more than 2, 3, 4, 5, 6, 7, 8, 9, 10, 15, 20, 25, 30 times higher than a ratio found in the cell not comprising said transgenic sequence of (a) and/or (b), and comprises, optionally, a matrix attachment region (MAR) element.

The invention is also directed at a recombinant eukaryotic, preferably mammalian, host cell, comprising
(a) a transgenic sequence expressing a NHEJ suppressor,
(b) a transgenic sequence expressing one or more HR enzyme or HR activator,
(c) a mutation inactivating or downregulating a NHEJ gene, and/or
(d) a mutation enhancing expression or activity of a HR enzyme, a HR activator or a NHEJ suppressor, and
   a transgene integrated into the genome of said cell, and optionally, a MAR element, wherein said MAR element is provided in cis or trans to said transgene.

The one or more HR enzymes may be Rad 51, Rad 52, RecA, Rad 54, RuvC or BRCA2 and/or the HR activator may be RS-1 and/or the NHEJ suppressor may be NU7026 and/or wortmannin.

The transgene may be functionally linked to a control element for inducible expression such as an inducible promoter, wherein said inducible promoter is optionally a promoter activated physically such as a heat shock promoter or chemically such as promoter activated a IPTG or Tetracycline.

The mutation(s) in (c) or (d) may be mutation(s) in a xrcc4 gene, RAD51 strand transferase gene, a DNA-dependent protein kinase gene, the Rad 52 gene, the RecA gene, the Rad 54 gene, the RuvC gene and/or the BRCA2 gene.

The transgene may be integrated into a single locus of the genome of the cell and may form a concatemeric structure. The concatemeric structure may comprise at least 200, 300, 400, 500 or 600 copies of the transgene.

The invention is also directed at a recombinant eukaryotic, preferably mammalian host cell, comprising
integrated into a single locus of the genome a concatemeric structure of a transgene functionally linked to a promoter, wherein the concatemeric structure comprises at least 300, 400, 500 or 600 copies of the transgene and at least one MAR element, wherein said MAR element is provided in cis or trans to said transgene, and wherein said cell is preferably part of a cell population that has been synchronized.

The at least one MAR may be provided in cis, the majority of said transgenes may be provided with a MAR for each of said transgenes and/or the transgene may be flanked by at least two of said MAR elements. The at least one MAR element may have at least 90% sequence identity with SEQ ID NOs: 1-3 or may be a variant of SEQ ID NOs: 1-3 and/or may be located upstream of a promoter/enhancer sequence of said transgene. The cell may be a CHO cell, a HEK 293 cell, a stem cell or a progenitor cell.

The invention is also directed at the use of any one of the recombinant eukaryotic host cells mentioned herein, in particular for the expression of said transgene.

The invention is also directed at a kit comprising
a. in a first container, a vector comprising optionally a MAR element and restriction sites for integration of a transgene into said vector,
b. in a second container, a recombinant eukaryotic host mentioned herein, and
c. instructions how to use said vector in transfecting said cell for transgene expression.

The kit may also contain a synchronizing agent or instructions on how to synchronize a cell population comprising said cell(s). The vector may be used to transfect the cell at least twice, each time when the majority of the cell of said cell population is at the G1 phase.

The invention is also directed at a non-primate recombinant eukaryotic host cell comprising

a transgenic sequence encoding at least one primate protein or a primate RNA involved in translocation across the ER membrane and/or secretion across the cytoplasmic membrane, such as a protein or a RNA of a signal recognition particle (SRP) or a protein of a secretory complex (translocon) or a subunit thereof.

The cell may further comprise a transgene functionally attached to a signal peptide coding sequence, wherein said transgene may be present in the cell in multiple copies, preferably in form of a concatemeric structure. The cell may comprise at least 200, 300, 400, 500 or 600 copies of the transgene. A signal peptide encoded by said signal peptide coding sequence may comprise a hydrophobic stretch of amino acids and may have one or more sequences for interacting with SRP54. The cell may also comprise an epigenetic regulator element, such as an MAR element, located in cis or trans to said transgene. The protein or RNA involved in the translocation across the ER membrane and/or secretion across the cytoplasmic membrane may be a protein or RNA of the SPR, in particular SPR9, SPR14, SPR19, SPR54, SPR68, SPR72 and/or 7SRNA. The protein of the SPR may be a human SPR14, preferably combined with one or more other of said proteins or RNA involved in the translocation across the ER membrane and/or secretion across the cytoplasmic membrane. The one or more other of said proteins may be human SR and/or human Translocon proteins. The protein of the SPR may be human SPR54, preferably combined with one or more other of said proteins or RNA involved in the translocation across the ER membrane and/or secretion across the cytoplasmic membrane. The one or more other of said proteins may be human SR and/or human Translocon proteins.

The protein or RNA involved in the secretion and/or translocation across the cytoplasmic membrane may be one of the proteins of the translocon, in particular Sec61αβγ, Sec62, Sec63 and/or a subunit thereof. The protein or RNA involved in the secretion and/or translocation across the cytoplasmic membrane may be a combination of SRP9, SR14 and a Translocon protein. The transgene may a immunoglobulin, a subunit or fragment thereof or a fusion protein. The non-primate cell may be a rodent cell, preferably a CHO cell. The signal sequence coding sequence may have at least 90% sequence identity with SEQ ID NOs: 4-11 or may be a variant of any one of said sequences.

The invention is also directed at the use of the non-primate recombinant eukaryotic host cells in the secretion and/or translocation of a transgene expression product across the cytoplasmic membrane of the cell.

The invention is also directed at a kit comprising
(a) in one container, non-primate recombinant host cell comprising, as part of the genome of the cell, a transgenic sequence encoding at least one protein or a RNA involved in translocation across the ER membrane and/or secretion across the cytoplasmic membrane, such as a protein or a RNA of a signal recognition particle (SRP) or a protein of a secretory complex (translocon) or a subunit thereof,
(b) in a separate container, at least one vector comprising restriction sites for integration of a transgene into said vector and optionally a MAR element, and
(c) instructions for expressing and secreting a transgene expression product of said transgene using said cell.

The invention is further directed at a method for protein secretion of a transgene comprising:
providing a non-primate eukaryotic host cell comprising
(a) a transgenic sequence encoding at least one primate protein or a primate RNA involved in secretion and/or translocation across the endoplasmic reticulum and/or the endoplasmic reticulum and/or the cytoplasmic membrane, such as a protein or a RNA of a signal recognition particle (SRP) or a protein of a secretory complex (translocon) or a subunit thereof, and
(b) a transgene functionally attached to a signal peptide coding sequence.

The transgenic sequence may increase a total amount of protein or RNA involved in secretion and/or translocation across the cytoplasmic membrane present in said cell by more than 10%, 20%, 30%, 40% 50%, 60%, 70%, 80%, 90% or 100% above a level found in the cell prior to comprising/expressing said transgenic sequence.

The transgene may be present in the cell as a concatemeric structure integrated into the genome of the cell, wherein the concatemeric structure preferably comprises at least 200, 300, 400, 500 or 600 copies of the transgene and may be integrated at a single locus of a genome of said cell.

A signal peptide encoded by the signal peptide coding sequence may comprise a hydrophobic stretch of amino acids and may have sequences for interacting with SRP54.

The transfection in (b) may be a subsequent transfection and may be preceded by an initial transfection with nucleic acid such as a vector or nucleic acid fragments.

The vector of the initial transfection may correspond to the vector in (b).

The transgenic sequence may have at least 90% sequence identity with a sequence selected from the group of SEQ ID NOs: 4-11 or may be a variant of any one of said sequences.

The invention is also directed at a method for identifying a protein secretion and/or translocation increasing activity of a transgenic sequence comprising:
monitoring a first mammalian cell comprising a transgene encoding a recombinant protein, wherein said recombinant protein is secreted by said cell at a first level,
monitoring a second mammalian cell comprising said transgene encoding said recombinant protein, wherein the recombinant protein is secreted by said cell at a second level,
wherein said second level exceeds said first level,
introducing into said first mammalian cell the transgenic sequence encoding at least one protein or a RNA involved in secretion and/or translocation across the cytoplasmic membrane, and
determining changes in the secretion level of said recombinant protein in said first cell,
wherein an increase beyond the first level identifies the protein secretion increasing activity of said transgenic sequence.

### BRIEF DESCRIPTION OF THE FIGURES

FIGS. 1(A) to (F): Analysis of the effect of MARs and successive transfections on gene transfer and expression and Illustration of transgene expression levels obtained by the double transfection of MAR-containing expression vectors.

FIG. 2 (A) to (D): Determination of the optimal timing between successive transfections and cell culture progression through the cell division cycle.

FIG. 3 (A) to (E): DNA transport, integration and expression upon successive transfections and relationship between mean GFP fluorescence and transgene copy number in monoclonal cell populations.

FIG. 4 (A) to (C): Subcellular distribution of transfected DNA and effect of DNA conformation on gene transfer and expression.

FIG. 5 (A) to (E): High transgene expression via MARS, plasmid homology and homologous recombination and model for improved expression by repeated transfection with MAR.

FIG. 6 (A) to (C): Characterization of the Heavy and Light chain of immunoglubilin expressed by high and low recombinant IgG-producers CHO clones.

FIG. 7: Characterization of the ER folding and UPR machineries of High and Low IgG-producers.

FIG. 8(A), (B): SRP14 transfection of recombinant IgG producing CHO clones abolished light chain aggregation and rescued IgG secretion.

FIG. 9: Increase in MAb production in CHO cell pools expressing various combinations of SRP9, SRP14, SRP54, SR and Translocon.

FIG. 10: Map of an expression vector showing the expression cassette for the transgene of interest which is flanked by two SGEs.

### DETAILED DESCRIPTION OF VARIOUS AND PREFERRED EMBODIMENTS

A transgene as used in the context of the present invention is an isolated and purified deoxyribonucleotide (DNA) sequence coding for a given mature protein (also referred to herein as a DNA encoding a protein) or for a precursor protein or a functional RNA. Some preferred transgenes according to the present invention are transgenes encoding immunoglobulins (Igs) and Fc-fusion proteins and other proteins, in particular proteins with therapeutical activity (“biotherapeutics”). As used herein, the term transgene shall, in the context of a DNA encoding a protein, not include untranscribed flanking regions such as RNA transcription initiation signals, polyadenylation addition sites, promoters or enhancers. Generally, the term transgene is used in the present context when referring to a DNA sequence that is introduced into a cell such as an eukaryotic host cell via transfection (the term also includes, in the context of the present invention, the process of introducing foreign DNA via a viral vector, which is also sometimes referred to as transduction) and which encodes the product of interest also referred to herein as the “transgene expression product” or “heterologous protein”. The transgene might be functionally attached to a signal peptide coding sequence, which encodes a signal peptide which in turn mediates and/or facilitates translocation and/or secretion across the endoplasmic reticulum and/or cytoplasmic membrane and is removed prior or during secretion. The term “transgenic sequence”, on the other hand is used, when referring to a DNA sequence that is introduced into a cell such as an eukaryotic host cell via transfection and which increase the expression and/or secretion of the product of interest. A transgenic sequence often encodes a protein or a RNA sequence. Transgenic sequences of the present invention are, e.g., those that specifically enhance HR (homologous recombination) or decrease non homologous end joining (NHEJ). Respective proteins are discussed in more detail below. Other “transgenic sequences” are those that encode protein(s) or RNA(s) involved in the processing, secretion and/or translocation across the endoplasmic reticulum and/or cytoplasmic membranes. The “transgenic sequences” may include non-translated control sequences.

An enhancement of the expression and/or secretion is measured relative to a value obtained from a control cell that does not comprise the respective transgenic sequence. Any statistically significant enhancement relative to the value of a control qualifies as a promotion.

The HR/NHEJ ratio (or HR/NHEJ activity ratio) is the ratio of HR (homologous recombination) to NHEJ (non homologous end joining) activity occurring in a cell such as a eukaryotic cell, e.g., a recombinant eukaryotic host cell. The HR/NHEJ ratio is generally measured in a cell population, that is, a group of, e.g., eukaryotic cells of the same kind, e.g., a CHO cell clone. When reference is made herein to, e.g., optimizing or enhancing (increasing), e.g., the HR/NHEJ ratio of a cell it is to be understood that the fact that such optimization or enhancement occurred in the respective cell population. The reference point for any such optimization or enhancement is the ratio that exists in a corresponding cell population in which no measures were performed to enhance or optimize HR/NHEJ ratio. This is, e.g., the parent cell population of said cell, i.e., the cell population from which the enhanced or optimized cell is derived. The HR/NHEJ ratio (or HR/NHEJ activity ratio) can be enhanced to exceed more than 2, 3, 4, 5, 6, 7, 8, 9, 10, 11, 12, 13, 14, 15 times that of the reference cell population, which may be referred to herein, e.g., as a “cell not comprising said transgenic sequence and/or not being mutated.” Optimization and enhancement measurements include treatments in which the cell is “treated” generally without being genetically modified. Such a treatment includes the simple measure of synchronizing the cell population so that, e.g., a majority of cells of the population are, at the time of transfection, in the G1 phase. Different methods are known to accomplish such a synchronization and include, but are not limited to, use of chemical agents (synchronizing chemicals) and low temperature. Golzio et al. (2002) describe the cell synchronization by subjecting the cells to a treatment with sodium butyrate. Grosjean et al. (2002) describe that a majority of cells are arrested at the border between the G1 and S-phase after administration of mimosine as synchronizing chemical. Bjursell et al. (1973) describe synchronizing CHO cells using thymidine.

HR has been reported to require a group of RAD51-related proteins (West 2003). Thus, HR can be enhanced by providing supplemental HR proteins (HR enzymes) to the cells, which include, e.g., Rad 51, Rad 52, RecA, Rad 54, RuvC or BRCA2. HR activators may also be employed. Those include, but are not limited to, RS-1 (RD51-stimulatory compound 1). RS-1 enhances the homologous recombination activity of hRAD51 by promoting the formation of active presynaptic filaments (Jayathilaka et al. 2008). NHEJ has been reported to involve, in mammalian cells, two protein complexes, the heterodimer Ku80-Ku70 associated with DNA-PKcs and ligase IV with its co-factor XRCC4 (Delacôte et al., 2002). Suppressors of the NHEJ, which may also employed in the context of the present invention, include NU7026 (2-(morpholin-4-yl)-benzo(h) chomen-4-one), a DNA-PK inhibitor. Suppression of the NHEJ function using the chemical NU7026 may facilitate access of DNA ends to an intact homologous recombination repair pathway (Yang et al. 2009). Another suppressor of NHEJ is Wortmannin, a PI3k inhibitor of p110 PI 3 kinase, which also inhibits DNA-dependent protein kinase, which is known to mediate DNA double strand repair (Boulton et al., 1996).

The HR/NHEJ ratio of a cell may be enhanced by overexpressing those HR enzymes, HR activators and/or NHEJ suppressors or by HR activating or NHEJ suppressing physical or chemical treatments. One way of accomplishing such an overexpression is by introducing a “transgenic sequence” encoding such enzymes etc. into the respective cell. Such a sequence is referred to as “transgenic sequence” to signify that it is not part of the corresponding unmodified cell. The transgenic sequence is often integrated into the genome of the cell.

The proteins described above, such as the HR enzymes, activators and/or the NHEJ suppressors may be expressed in the modified cell inductively or constitutively. A person skilled can readily ascertain the appropriate vector constructions that allow for an inductive or constitutive expression.

Similarly, cells have been modified by mutation to enhance HR and/or decrease NHEJ and/or enhance the HR/NHEJ ratio of a cell. Several publications describe the inhibition of the NHEJ pathway, the pathway responsible for random integration of polynucleotides in cells, as a method for improving the HR/NHEJ ratio (see for example Krappmann et al., 2006). Genes and/or proteins that can be inactivated to block NHEJ include Ku80, Ku70, Ligase IV or XRCC4 (see also reference herein to the V3.3 mutant) and may, in the context of the present invention, result in very significant enhancements of the HR/NHEJ ratio and improvement of transgene expression, such as up to 5, 6, 7, 8, 9, 10, 15, 20, 25, 30, 35, 40, 45, 50 or even up to 60-fold increase on average of transgene expression. Similarly, certain mutations may enhance HR by, e.g., enhancing the expression of certain endogenous HR enzymes or activators of a cell.

A HR/NHEJ peak (or HR/NHEJ activity peak) is a period during the cell cycle of a cell population of eukaryotic cells at which HR/NHEJ ratio is elevated and peaks. If, in context of the present invention, reference is made to a HR/NHEJ peak of a cell, it is understood that reference is made to a cell of a cell population of the same kind, e.g., a cell population modified by a transgenic sequence to express a HR enzyme. The “HR/NHEJ peak” encompasses a time interval around the highest HR/NHEJ elevation (the tip of the peak, peak tip) in a graph plotting time against a value representing HR/NHEJ or just HR. The preferred time interval for a transfection is before the HR/NHEJ peak (e.g. at the G1 phase of the cell cycle), so that DNA reaches the cell nucleus as the time around the tip of the peak (peak tip, e.g. late S and G2 phases), defined by the point in time at which a 50% rise or more of the HR/NHEJ (or just HR) from the value at which the line towards the tip of the peak starts to rise (“bottom value”) to the tip of the peak has been reached. A peak comes into existence, e.g., when a minimum number of cells in the cell population are in the G1, or early S phase, a phase when HR activity is known to be low, and/or when the majority of cells are in the S phase or in the G2 phase, when HR activity is known to be highest.

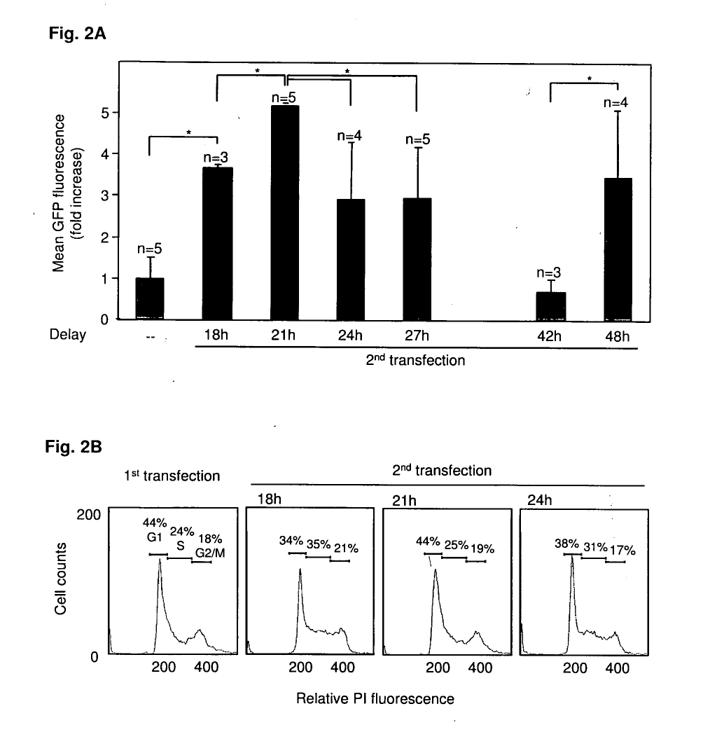

A point in time when the majority of the cells of a cell population are in the G1 phase is also demonstrated in the graphs shown in FIG. 2(B). Here the percentage of the population associated with each cell cycle state (G1, S, G2/M) is indicated. As can be seen from this Figure more than 80%, more than 85%, more than 90% or even more than 95% of the cells of the population depicted were identified to be either in G1, S or G2/M phase. Of the cells found to be in one of these phases, the majority was found, in this example, to be in the G1 phase after 21 hours. The percentile of G1 phase cells was thus highest compared to the percentile of S or G2/M phase cells.

A functional RNA includes any type of RNA that produces a direct or indirect effect in the cell that differs from being translated into a protein. Typical examples are antisense RNAs or small interfering RNAs (si RNAs).

An eukaryotic host cell is a cell that does or is designed to “host” a transgene according to the present invention. A recombinant eukaryotic host cell is genetically modified, that is contains additional sequences, either as part of its genome or as part of an extrachromosomal element, such as a vectors, generally to enhance expression or secretion of the transgene expression product.

A concatemer or concatemeric structure is a long continuous DNA stretch or molecule that contains multiple copies of the same monomeric DNA sequences linked in series. In the context of the present invention the monomeric DNA sequence is or comprises often a transgene. The concatemeric structures of the transgene which might include, e.g., promoter and enhancer sequences, generally integrate into the genome of the host cell. This integration can happen at multiple locations (loci) (integration sites) of the chromosome of the host or at a single locus. A single concatemeric structure might include more than 200, 300, 400, 500, 600, 700 or more than 800 monomeric DNA sequences comprising said transgene. A head-to-tail array of the monomeric DNA sequences is preferentially observed. Transgenes that are said to be present in a cell in multiple copies may have a concatemeric structure.

A MAR element, a MAR construct, a MAR sequence, a S/MAR or just a MAR according to the present invention is a nucleotide sequence sharing one or more (such as two, three or four) characteristics with a naturally occurring “SAR” or “MAR” and having at least one property that facilitates protein expression of any gene influenced by said MAR. A MAR element has also the feature of being an isolated and/or purified nucleic acid with MAR activity, in particular, with transcription modulation, preferably enhancement activity, but also with, e.g., expression stabilization activity and/or other activities which are also described under “enhanced MAR constructs.” MAR elements belong to a wider group of epigenetic regulator elements which also include boundary or insulator elements, locus control regions (LCRs), stabilizing and antirepressor (STAR) elements, and ubiquitously acting chromatin opening (UCOE) elements. MAR elements may be defined based on the identified MAR they are primarily based on: A MAR S4 construct is, accordingly, a MAR elements that whose majority of nucleotide (50% plus) are based on MAR S4. Several simple sequence motifs high in A and T content have often been found within SARs and/or MARs, but for the most part, their functional importance and potential mode of action has been unresolved. These include the A-box, the T-box, DNA unwinding motifs, SATB1 binding sites (H-box, A/T/C25) and consensus topoisomerase II sites for vertebrates or Drosophila.

An AT/TA-dinucleotide rich bent DNA region (hereinafter referred to as “AT-rich region”) as commonly found in MAR elements is a bent DNA region comprising a high number of A and Ts, in particular in form of the dinucleotides AT and TA. In a preferred embodiment, it contains at least 10% of dinucleotide TA, and/or at least 12% of dinucleotide AT on a stretch of 100 contiguous base pairs, preferably at least 33% of dinucleotide TA, and/or at least 33% of dinucleotide AT on a stretch of 100 contiguous base pairs (or on a respective shorter stretch when the AT-rich region is of shorter length), while having a bent secondary structure. However, the “AT-rich regions” may be as short as about 30 nucleotides or less, but is preferably about 50 nucleotides, about 75 nucleotides, about 100 nucleotides, about 150, about 200, about 250, about 300, about 350 or about 400 nucleotides long or longer.

Some binding sites are also often have relatively high A and T content such as the SATB1 binding sites (H-box, A/T/C25) and consensus Topoisomerase II sites for vertebrates (RNYNNCNNGYNGKTNYNY) or Drosophila (GTNWAYATTNATNNR). However, a binding site region (module), in particular a TFBS region, which comprises a cluster of binding sites, can be readily distinguished from AT and TA dinucleotides rich regions (“AT-rich regions”) from MAR elements high in A and T content by a comparison of the bending pattern of the regions. For example, for human MAR 1_{—}68, the latter might have an average degree of curvature exceeding about 3.8 or about 4.0, while a TFBS region might have an average degree of curvature below about 3.5 or about 3.3. Regions of an identified MAR can also be ascertained by alternative means, such as, but not limited to, relative melting temperatures, as described elsewhere herein. However, such values are specie specific and thus may vary from specie to specie, and may, e.g., be lower. Thus, the respective AT and TA dinucleotides rich regions may have lower degrees of curvature such as from about 3.2 to about 3.4 or from about 3.4 to about 3.6 or from about 3.6 to about 3.8, and the TFBS regions may have proportionally lower degrees of curvatures, such a below about 2.7, below about 2.9, below about 3.1, below about 3.3. In SMAR Scan II, respectively lower window sizes will be selected by the skilled artisan.

The terms MAR element, MAR construct, a MAR sequence, a S/MAR or just a MAR also includes enhanced MAR constructs that have properties that constitute an enhancement over an natural occurring and/or identified MAR on which a MAR construct according to the present invention may be based. Such properties include, but are not limited to, reduced length relative to the full length natural occurring and/or identified MAR, gene expression/transcription enhancement, enhancement of stability of expression, tissue specificity, inducibility or a combination thereof. Accordingly, a MAR element that is enhanced may, e.g., comprise less than about 90%, preferably less than about 80%, even more preferably less than about 70%, less than about 60%, or less than about 50% of the number of nucleotides of an identified MAR sequence. A MAR element may enhance gene expression and/or transcription of a transgene upon transformation of an appropriate cell with said construct.

A MAR element is preferably inserted upstream of a promoter region to which a gene of interest is or can be operably linked. However, in certain embodiments, it is advantageous that a MAR element is located upstream as well as downstream or just downstream of a gene/nucleotide acid sequence of interest. Other multiple MAR arrangements both in cis and/or in trans are also within the scope of the present invention.

The present invention is also directed to uses of MAR elements combined with one or more non-MAR epigenetic regulators such as, but not limited to, histone modifiers such as histone deacetylase (HDAC), other DNA elements (epigenetic regulator elements) such as locus control regions (LCRs), insulators such as cHS4 or antirepressor elements (e.g., stabilizer and antirepressor elements (STAR or UCOE elements) or hot spots (Kwaks THJ and Otte AP).

Synthetic, when used in the context of a MAR/MAR element refers to a MAR whose design involved more than simple reshuffling, duplication and/or deletion of sequences/regions or partial regions, of identified MARs or MARs based thereon. In particular, synthetic MARs/MAR elements generally comprise one or more, preferably one, region of an identified MAR, which, however, might in certain embodiment be synthesized or modified, as well as specifically designed, well characterized elements, such as a single or a series of TFBSs, which are, in a preferred embodiment, produced synthetically. These designer elements are in many embodiments relatively short, in particular, they are generally not more than about 300 bps long, preferably not more than about 100, about 50, about 40, about 30, about 20 or about 10 bps long. These elements may, in certain embodiments, be multimerized. Such synthetic MAR elements are also part of the present invention and it is to be understood that generally the present description can be understood that anything that is said to apply to a “MAR element” equally applies to a synthetic MAR element.

Functional fragments of nucleotide sequences of identified MAR elements are also included in the above definition as long as they maintain functions of a MAR element as described above.

Some preferred identified MAR elements include, but are not limited to, MAR 1_{—}68, MAR X_{—}29, MAR 1_{—}6, MAR S4, MAR S46 including all their permutations as disclosed in WO2005040377 and US patent publication 20070178469, which are specifically incorporated by reference into the present application for the disclosure of the sequences of these and other MAR elements. The chicken lysozyme MAR is also a preferred embodiment (see, U.S. Pat. No. 7,129,062, which is also specifically incorporated herein for its disclosure of MAR elements).

Cis refers to the placement of two or more elements (such as chromatin elements) on the same nucleic acid molecule such as, but not limited to, the same vector or chromosome.

Trans refers to the placement of two or more elements (such as chromatin elements) on the two or more nucleic acid molecules such as, but not limited to, two or more vectors or chromosomes.

A sequence is said to act in cis and/or trans on, e.g., a gene when it exerts its activity from a cis/trans location.

A transgene or transgenic sequence of the present invention is often part of a vector. A vector according to the present invention is a nucleic acid molecule capable of transporting another nucleic acid, such as a transgene that is to be expressed by this vector, to which it has been linked, generally into which it has been integrated. For example, a plasmid is a type of vector, a retrovirus or lentivirus is another type of vector. In a preferred embodiment of the invention, the vector is linearized prior to transfection.

The vector sequence of a vector is the DNA or RNA sequence of the vector excluding any “other” nucleic acids such as transgenes as well as genetic elements such as MAR elements.

When the present specification refers to “plasmid” or “vector” homology, the term refers to the homology (herein used synonymous with sequence identity) of the entire plasmid or vector including MARs and genes.

An eukaryotic, including a mammalian cell, such as a recombinant eukaryotic host cell, according to the present invention is capable of being maintained under cell culture conditions. Non-limiting examples of this type of cell are non-primate eukaryotic host cells such as Chinese hamster ovary (CHOs) cells and baby hamster kidney cells (BHK, ATCC CCL 10). Primate eukaryotic host cells include, e.g., human cervical carcinoma cells (HELA, ATCC CCL 2) and monkey kidney CV1 line transformed with SV40 (COS-7, ATCC CRL-1587). A recombinant eukaryotic host cell signifies a cell that has been modified, e.g., by transfection with transgenic sequence and/or by mutation. The eukaryotic host cells are able to perform post-transcriptional modifications of proteins expressed by said cells. In certain embodiments of the present invention, the cellular counterpart of the eukaryotic (e.g., non-primate) host cell is fully functional, i.e., has not been, e.g., inactivated by mutation. Rather the transgenic sequence (e.g., primate) is expressed in addition to its cellular counterpart (e.g., non-primate).

Transfection according to the present invention is the introduction of a nucleic acid into a recipient eukaryotic cell, such as, but not limited to, by electroporation, lipofection, via a viral vector (sometimes referred to as “transduction”) or via chemical means including those involving polycationic lipids.

Transformation as used herein, refers to modifying an eukaryotic cell by the addition of a nucleic acid. For example, a transformed a cell includes a cell that that has been transfected with a transgenic sequence, e.g., via electroporation of a vector comprising this sequence. However, in many embodiments of the invention, the way of introducing the transgenic sequences of the present invention into a cell, is not limited to any particular method.

A single transfection means that the described transfection is only performed once.

Transcription means the synthesis of RNA from a DNA template. “Transcriptionally active” refers to a transgene that is being transcribed.

Identity means the degree of sequence relatedness between two nucleotide sequences as determined by the identity of the match between two strings of such sequences, such as the full and complete sequence. Identity can be readily calculated. While there exists a number of methods to measure identity between two nucleotide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a nucleic acid comprising a nucleotide sequence having at least, for example, 95% “identity” with a reference nucleotide sequence means that the nucleotide sequence of the nucleic acid is identical to the reference sequence except that the nucleotide sequence may include up to five point mutations per each 100 nucleotides of the reference nucleotide sequence. In other words, to obtain a nucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Sequence identities of more about 60%, about 70%, about 75%, about 85% or about 90% are also within the scope of the present invention.

A nucleic acid sequence having substantial identity to another nucleic acid sequence refers to a sequence having point mutations, deletions or additions in its sequence that have no or marginal influence on the respective method described and is often reflected by one, two, three or four mutations in 100 bps.

The invention is directed to both polynucleotide and polypeptide variants. A “variant” refers to a polynucleotide or polypeptide differing from the polynucleotide or polypeptide of the present invention, but retaining essential properties thereof. Generally, variants are overall closely similar and in many regions, identical to the polynucleotide or polypeptide of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred.

The invention also encompasses allelic variants of said polynucleotides. An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

A promoter sequence or just promoter is a nucleic acid sequence which is recognized by a host cell for expression of a specific nucleic acid sequence. The promoter sequence contains transcriptional control sequences which regulate the expression of the polynucleotide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell. The promoter is “functionally linked” to a specific nucleic acid sequence if it exercises its function on said promoter.

Enhancers are cis-acting elements of DNA, usually from about 10 to about 300 nucleotides long that act on a promoter to increase its transcription. Enhancers from globin, elastase, albumin, alpha-fetoprotein, and insulin enhancers may be used. However, an enhancer from a virus may be used; examples include SV40 on the late side of the replication origin, the cytomegalovirus early promoter enhancer, the polyoma enhancer on the late side of the replication origin and adenovirus enhancers.

Exponentially as used herein is not an exact mathematical term, but describes a biological growth curve of cells, wherein a graph of such a growth is not as a straight line, but is a curve that points upwards and, at least over a certain period of time, continuously becomes steeper. In any event, it connotes a more than additive, e.g. increase.

A variability of expression as used in the context of the present invention refers to the variability in expression of one transformed cell versus another transformed cell of the same kind. This variability is a result of differing transgene copies and/or the site of transgene integration. Also, the co-integration of multiple copies of a transgene at the same locus may lead to silencing and thus contribute to the variability.

Moreover, the term comprise and derivations thereof do not exclude other elements or steps. Furthermore, the indefinite article “a” and derivations thereof do not exclude a plurality. The functions of several of the features mentioned in the claims can be fulfilled by a unity. The terms substantially, about, approximately and the like in connection with a characteristic or a value in particular also define exactly this characteristic or exactly this value.

### Homologous Recombination (HR), Non-Homologous End-Joining (NHEJ) and the Reduction of Variability of Transgene Expression

The variability in transgene expression among independent transformants is influenced by the number of genes stably integrated in the genome of the cells and by the site of integration. While studying MARs and in particular while determining why MARs yield higher expression of transgenes, several observations were made that resulted in broader, MAR independent, finding:

First, via quantitative PCR it could be proven that MAR elements increase the number of transgene copies integrated in the genome. These results substantiated previous semi-quantitative observations (Kim et al., 2004; Girod et al., 2005). In addition, fluorescent in situ hybridization analysis of metaphase chromosomes of stable cell pools showed higher intensity of fluorescence in cells transfected with MARs, thus confirming the increase of transgene integration.

Further investigations, resulted in the finding that, after a single transfection with a transgene and a respective MAR, the MAR excreted no significant effect on the amount of plasmids transported to the cell nuclei after transfection. A possible explanation was that concatemerization and/or integration explained the high copy number integrated in the genome of the cells. The initial idea was that MARs may play a role as DNA recombination signals. Because of their structural properties, such as their unwinding and unpairing potential, the possibility existed that they could augment the frequency of homologous recombination between transfected plasmids, thus allowing the formation of bigger concatemers and integration of high number of plasmid copies.

Secondly, the phenomenon was investigated that, under certain circumstances, two successive transfections (a single initial transfection and subsequent ones) with a MAR next to the transgene allow a more than additive increase in transgene expression rather than providing just an additive, e.g., two-fold increase, that one would expect from e.g. two independent transfections.

Via quantitative PCR it was demonstrated that the high transgene expression associated with successive MAR transfections was based on a similar high increase of the number of integrated transgene copies after multiple transfection events as compared to one single event.

While there was an increase in number of plasmids that entered the nucleus, it was further investigated whether the high number of integrated transgene copies in successive MAR transfections may, at least in part, be due to better concatemerization between homologous plasmids introduced into the cells during the successive transfections. A process involving homologous recombination was suspected and tested by studying the effect of plasmid homology. In particular, double transfections were performed with different combinations of transgenes, plasmid backbones and/or MARs. FACS analyses reveal that high plasmid homology (vector sequence homology and transgene as well as MAR homology) was generally required for higher efficiency of integration and transgene expression. Changing either the gene of expression and vector sequence or MARs reduced both the efficiency of integration and transgene expression. In fact, the observed effect of double transfection was totally abolished when all sequences differed.

In addition, the timing between successive transfections was shown to be very important to achieve the optimal protein expression. It was hypothesized that, at the time of the second transfection, cells should be in a cell cycle state that is favorable for a higher recombination rate, leading to a formation of bigger concatamers and integration of more plasmids into the host genome. As discussed above, the concatermierzation of transgenes may result from two principal mechanisms that exist in an eukaryotic cell, one is HR and the other is NHEJ. Thus, the effect one or the other on the double transfection were tested. For this, different CHO mutants were used that were either deficient in non-homologous end-joining or homologous recombination. It was found that the NHEJ pathway antagonized efficient transgene integration and expression, while a functional homologous recombination pathway and homologous DNA sequences on the transfected vectors favored high-level expression. When mutant CHO cells that relied solely on homologous recombination were used, transfections of MAR-containing vector yielded a very high increase in the level of transgene expression as compared to non-mutated cells. Also, FISH analysis did not show any multiple integration events with successive transfections at specific time intervals, here 21 hours, indicating that all transgene integrated at one chromosomal locus.

Thus, the effect of the host cells deficient in non-homologous end-joining or homologous recombination on integration of the MAR influenced transgene, led to a broader concept, unrelated to MARs, namely that transgene integration is favored by HR and disfavored by NHEJ. Thus, we devised methods and constructs that take advantage of this finding to increase transgene integration and thus transgene expression. The methods include method to increase the HR, decrease the NHEJ and/or increase the HR/NHEJ ratio at the time of integration with treatments, such a chemical or temperature treatments or other treatments that that allow a synchronization of a cell population. Other treatments and modifications are described above under the discussion of the HR/NHEJ ratio. The constructs were primarily cells having the suitable makeup to allow for a HR enhancement, a NHEJ decrease and/or an enhancement of the HR/NHEJ ration.

The decrease the NHEJ and/or the enhancement of HR or the HR/NHEJ ratio in the host cell has also a particularly advantageous effect in the context of successive transfections which may or may not involve a MAR.

However, while the MAR independent process provide considerable progress in terms of transgene integration and expression, MARs and other epigenetic regulators still provide further advantageous properties that in part can be explained by favorably influencing homologous recombination as well as by other mechanisms, some of which have been discussed previously (see, e.g., US Patent Publication US 2007/0178469).

MAR elements have been described to have the ability to improve transgene expression by reducing population expressing low level of protein by protecting transgenes from the silencing effects, which likely result from the integration in non-permissive heterochromatic loci (Bell and Felsenberg, 1999). The anti-silencing effect observed in the presence of MAR may be mediated by chromatin modifications such as histone hyperacetylation at the site of transgene integration (Recillas-Targa et al., 2002; Yasui et al., 2002) or changes in subnuclear localization. Additionally, MARs may recruit regulatory proteins that modify chromatin to adopt a more transcriptionally permissive state, or they can recruit transcription factors that activate gene expression (Yasui et al., 2002; Hart and Laemmli, 1998). Alternatively, but not exclusively, MAR may recruit proteins to remodel chromatin structure towards an open state more permissive for integration events. Also the transcription of transgenes can be improved by an activation of the transgene promoter or enhancer by MAR. MAR may also favor integration in a permissive locus within the chromosome. Finally, they may enhance the transgene copy number integrated in the host genome by a mechanism unrelated to HR.

In this context, it should be noted that the perception that a higher copy number always supports stronger expression of a transgene is not necessarily valid since the presence of multiple copies integrated into the host genome favors silencing, resulting from the propensity of repeated elements to pair and assemble in heterochromatin. Alternatively, expression of repeated genes may lead to the formation of double-stranded and/or small interfering RNAs, which in turn may lead to epigenetic silencing. However, in the context of the present invention, it could be shown that the transgene copy number and cell fluorescence levels were shown to correlate well in the presence of MAR. Thus, an increase in transgene expression is likely not only to result from the integration of more transgene copies in the genome of cells, but to be favored by MAR-mediated inhibition of epigenetic silencing events that are associated with the integration of tandem gene copies.

As noted above, especially in the context of successive transfections, the increase in transgene integration/expression in the experiments performed, could be in part explained it by quantifying the amount of transgenes transported in the nuclei. Indeed, it could be shown that cell nuclei receive more plasmids with two transfections, in particular with MARs, and particularly during the second transfection, since the first transfection may facilitate DNA uptake and nuclear transport by the cells during the second transfection. Indeed, by assessing the intracellular trafficking of the DNA and quantifying the percentile of labeled pDNA in cellular organelles such as lysosomes, nuclei and cytosol after each transfection, it could be shown that plasmid DNA bearing a MAR seemed to escape lysosome degradation and to enter the nucleus during the second transfection much more efficiently. An explanation might be that the plasmids, in particular those of the first transfection, may saturate the cellular degradation machinery, thus allowing a more efficient DNA transport to the nucleus during the second transfection.

Thus, combining cells having an enhanced HR/NHEJ ratio, enhanced HR and/or a decreased NHEJ with MAR elements can be highly effective and is part of the present invention.

### The Mechanisms of Homologous Recombination (HR) and Non-Homologous End-Joining (NHEJ)

Transgenes use the recombination machineries to integrate at a double strand break into the host genome.

Double-strand breaks (DSBs) are the biologically most deleterious type of genomic damage potentially leading to cell death or a wide variety of genetic rearrangements. Accurate repair is essential for the successful maintenance and propagation of the genetic information.

There are two major DSB repair mechanisms: non-homologous end-joining (NHEJ) and homologous recombination (HR). Homologous recombination is a process for genetic exchange between DNA sequences that share homology and is operative only the S/G2 phases of the cell cycle, while NHEJ simply pieces together two broken DNA ends, usually with no sequence homology, and it functions in all phases of the cell cycle but is of particular importance during G0-G1 and early S-phase of mitotic cells (Wong and Capecchi, 1985; Delacote and Lopez, 2008). In vertebrates, HR and NHEJ differentially contribute to DSB repair, depending on the nature of the DSB and the phase of the cell cycle (Takata et al., 1998).

### NHEJ: Basic Mechanisms

Conceptually, the molecular mechanism of the NHEJ process seems to be simple: 1) a set of enzymes capture the broken DNA molecule, 2) a molecular bridge that brings the two DNA ends together is formed and 3) the broken molecules are re-ligated. To perform such reactions, the NHEJ machinery in mammalian cells involves two protein complexes, the heterodimer Ku80/Ku70 associated with DNA-PKcs (catalytic subunit of DNA-dependent protein kinase) and DNA ligase IV with its co-factor XRCC4 (X-ray-complementing Chinese hamster gene 4) and many protein factors, such as Artemis and XLF (XRCC4-like factor; or Cernunnos) (Delacote et al., 2002). NHEJ is frequently considered as the error-prone DSB repair because it simply pieces together two broken DNA ends, usually with no sequence homology and it generates small insertions and deletions (Moore and Haber, 1996; Wilson et al., 1999). NHEJ provides a mechanism for the repair of DSBs throughout the cell cycle, but is of particular importance during G0-G1 and early S-phase of mitotic cells (Takata et al., 1998; Delacote and Lopez, 2008). The repair of DSBs by NHEJ is observed in organisms ranging from bacteria to mammals, indicating that it has been conserved during evolution.

After DSB formation the key step in NHEJ repair pathway is the physical juxtaposition of the broken DNA ends. NHEJ is initiated by the association of the Ku70/80 heterodimer protein complex to both ends of the broken DNA molecule to capture, tether the ends together and create a scaffold for the assembly of the other NHEJ key factors. The DNA-bound Ku heterodimer complex recruits DNA-PKcs to the DSB, a 460 kDa protein belonging to the PIKK (phosphoinositide 3-kinase-like family of protein kinases) (Gottlieb and Jackson, 1993) and activates its serine/threonine kinase function (Yaneva et al., 1997). Two DNA-PKcs molecules interact together across the DSB, thus forming a molecular bridge between both broken DNA ends and inhibit their degradation (DeFazio et al., 2002). Then, DNA ends can be directly ligated, although the majority of termini generated from DSB have to be properly processed prior to ligation (Nikjoo et al., 1998). Depending of the nature of the break, the action of different combinations of processing enzymes may be required to generate compatible overhangs, by filling gaps, removing damaged DNA or secondary structures surrounding the break. This step in the NHEJ process is considered to be responsible for the occasional loss of nucleotides associated with NHEJ repair. One key end-processing enzyme in mammalian NHEJ is Artemis, a member of the metallo-β-lactamase superfamily of enzymes, which was discovered as the mutated gene in the majority of radiosensitive severe combined immunodeficiency (SCID) patients (Moshous et al., 2001). Artemis has both a 5′→3′ exonuclease activity and a DNA-PKcs-dependent endonuclease activity towards DNA-containing ds-ss transitions and DNA hairpins (Ma et al., 2002). Its activity is also regulated by ATM. Thus, Artemis seems likely to be involved in multiple DNA-damage responses. However, only a subset of DNA lesions seem to be repaired by Artemis, as no major defect in DSB repair were observed in Artemis-lacking cells (Wang et al., 2005, Darroudi et al., 2007).

DNA gaps must be filled in to enable the repair. Addition of nucleotides to a DSB is restricted to polymerases μ and λ (Lee et al., 2004; Capp et al., 2007). By interaction with XRCC4, polynucleotide kinase (PNK) is also recruited to DNA ends to permit both DNA polymerization and ligation (Koch et al., 2004). Finally, NHEJ is completed by ligation of the DNA ends, a step carried out by a complex containing XRCC4, DNA ligase IV and XLF (Grawunder et al., 1997). Other ligases can partially substitute DNA ligase IV, because NHEJ can occur in the absence of XRCC4 and Ligase IV (Yan et al., 2008). Furthermore, studies showed that XRCC4 and Ligase IV do not have roles outside of NHEJ, whereas in contrast, KU acts in other processes such as transcription, apoptosis, and responses to microenvironment (Monferran et al., 2004; Müller et al., 2005; Downs and Jackson, 2004).

As the person skilled in the art will readily understand, any mutation in or around one of the genes of the above referenced proteins (e.g., the heterodimer Ku80/Ku70, DNA-PKcs, but in particular DNA ligase IV, XRCC4, Artemis and XLF (XRCC4-like factor; or Cernunnos), PIKK (phosphoinositide 3-kinase-like family of protein kinases), that will decrease or shut down the NHEJ is within the scope of the present invention. Similarly, any protein or transgenic sequence acting on any one of the above pathways to decrease it or shut it down is within the scope of the present invention.

### HR: Basic Mechanisms

Homologous recombination (HR) is a very accurate repair mechanism. A homologous chromatid serves as a template for the repair of the broken strand. HR takes place during the S and G2 phases of the cell cycle, when the sister chromatids are available. Classical HR is mainly characterized by three steps: 1) resection of the 5′ of the broken ends, 2) strand invasion and exchange with a homologous DNA duplex, and 3) resolution of recombination intermediates. Different pathways can complete DSB repair, depending on the ability to perform strand invasion, and include the synthesis-dependent strand-annealing (SDSA) pathway, the classical double-strand break repair (DSBR) (Szostak et al, 1983), the break-induced replication (BIR), and, alternatively, the single-strand annealing (SSA) pathway. All HR mechanisms are interconnected and share many enzymatic steps.

The first step of all HR reactions corresponds to the resection of the 5′-ended broken DNA strand by nucleases with the help of the MRN complex (MRE11, RAD50, NBN (previously NBS1, for Nijmegen breakage syndrome 1)) and CtIP (CtBP-interacting protein) (Sun et al., 1991; White and Haber, 1990). The resulting generation of a 3′ single-stranded DSB is able to search for a homologous sequence. The invasion of the homologous duplex is performed by a nucleofilament composed of the 3′ ss-DNA coated with the RAD51 recombinase protein (Benson et al., 1994). The requirement of the replication protein A (RPA), an heterotrimeric ssDNA-binding protein, involved in DNA metabolic processes linked to ssDNA in eukaryotes (Wold, 1997), is necessary for the assembly of the RAD51-filament (Song and sung, 2000). Then RAD51 interacts with RAD52, which has a ring-like structure (Shen et al., 1996) to displace RPA molecules and facilitate RAD51 loading (Song and sung, 2000). Rad52 is important for recombination processes in yeast (Symington, 2002). However, in vertebrates, BRCA2 (breast cancer type 2 susceptibility protein) rather than RAD52 seems to play an important role in strand invasion and exchange (Davies and Pellegrini, 2007; Esashi et al., 2007). RAD51/RAD52 interaction is stabilized by the binding of RAD54. RAD54 plays also a role in the maturation of recombination intermediates after D-loop formation (Bugreev et al., 2007). In the other hand, BRCA1 (breast cancer 1) interacts with BARD1 (BRCA1 associated RING domain 1) and BACH1 (BTB and CNC homology 1) to perform ligase and helicase DSB repair activity, respectively (Greenberg et al., 2006). BRCA1 also interacts with CtIP in a CDK-dependent manner and undergoes ubiquitination in response to DNA damage (Limbo et al., 2007). As a consequence, BRCA1, CtIP and the MRN complex play a role in the activation of HR-mediated repair of DNA in the S and G2 phases of the cell cycle.

The invasion of the nucleofilament results in the formation of a heteroduplex called displacement-loop (D-loop) and involves the displacement of one strand of the duplex by the invasive strand and the pairing with the other. Then, several HR pathways can complete the repair, using the homologous sequence as template to replace the sequence surrounding the DSB. Depending of the mechanism used, reciprocal exchanges (crossovers) between the homologous template and the broken DNA molecule may be or may not be associated to HR repair. Crossovers may have important genetic consequences, such as genome rearrangements or loss of heterozygosity.

As the person skilled in the art will readily understand any mutation in or around one of the genes of the above referenced proteins (e.g., proteins of the MRN complex (MRE11, RAD50, NBN (previously NBS1, for Nijmegen breakage syndrome 1)) and CtIP (CtBP-interacting protein), RAD51, the replication protein A (RPA), Rad52, BRCA2 (breast cancer type 2 susceptibility protein), RAD54, BRCA1 (breast cancer 1) interacts with BARD1 (BRCA1 associated RING domain 1), BACH1 (BTB and CNC homology 1)) that will enhance HR is within the scope of the present invention. Similarly, any protein or transgenic sequence acting on any one of the above pathways to enhance it is within the scope of the present invention.

### The Choice Between DNA DSB-Repair Pathways

NHEJ and HR appear to be the two main eukaryotic DSB-repair pathways. Nevertheless, the balance between them differs widely among species. Vertebrate cells use NHEJ more frequently than yeast. One explanation is that the complexity of higher eukaryotic genomes makes the search for homology necessary for HR more difficult. In addition, the high level of repetitiveness may be dangerous for genetic stability if case of ectopic recombination. Alternatively, some factors, such as DNA-PKcs, BRCA1 and Artemis are found in vertebrates but not in yeast.

In mammals, it is known that NHEJ and HR operate in both competitive and collaborative manners, and studies on rodent cells and human cancer cell lines have shown that the choice between NHEJ and HR pathways depends on cell cycle stages. NHEJ provides a mechanism for the repair of DSBs throughout the cell cycle, but is of particular importance during G0-G1 and early S-phase of mitotic cells (Takata et al., 1998; Delacote and Lopez, 2008), whereas HR is active in late S/G2 phases. Several factors are also important in the regulation of the choice between both pathways, including regulated expression and phosphorylation of repair proteins, chromatin accessibility for repair factors, and the availability of homologous repair templates. A key factor that regulates HR efficiency is template availability. It is thus not surprising that cells upregulate HR during S and G2 phases of the cell cycle when sister chromatids are available because they are the favorite template for HR (Dronkert et al., 2000). This preference can be explained by an effect of proximity between sister chromatids from the time they form in S phase until they segregate in anaphase. But the presence of a homologous template is not sufficient for HR competence. Increasing evidence indicates that the shift from NHEJ toward HR as cells progress from G1 to S/G2 is actively regulated.

Indeed, HR is tightly regulated by CDK-dependent cell cycle controls in mammalian cells. It has been demonstrated that CDK-mediated phosphorylation of serine 3291 of BRCA2 blocks its interaction with RAD51 in M and early G1 phases. This phosphorylation represents one of the mechanisms by which HR is down-regulated (Esashi et al., 2007). Additionally, a fundamental difference between HR and NHEJ is that HR-mediated repair requires DNA resection (approx. 100-200 nucleotides) for homology searching and strand invasion (Sung and Klein, 2006). It is now clear DNA 5′-ended resection, is a key step that contributes to the choice of DSB repair, by initiating HR and inhibiting further possibilities of NHEJ. Resection depends on CDK1 activity. Interestingly, blocking CDK1 led to the persistence of MRE1 at the DSB site, suggesting that CDK1 activity is required for the regulation of end resection, rather than for MRN recruitment to broken ends (Ira et al., 2004). Finally, RAD51 and RAD52 expressions increase during S phase and contribute to HR activation (Chen et al., 1997). In contrast, NHEJ is down-regulated by the decrease of DNA-PK activity in S phase (Lee et al., 1997).

### Proteins at the HR/NHEJ Interface

The regulation of the choice between repair pathways may be controlled by the early acting proteins that act in both repair pathways. MRN complex and ATM are among them, and along with their mediator and transducer proteins form an efficient network that senses and signals any DNA damage. This network starts working very fast after the damage and is switch off soon after the task is accomplished.

The MRN complex is involved in DNA repair mechanisms, such as HR, NHEJ, DNA replication, telomere maintenance and in the signalling to the cell cycle checkpoints (D'Amours and Jackson, 2002; van den Bosch et al., 2003). The first step in DNA damage repair is the association of MRN complex as a heterotetramer (M2R2) with the broken ends of DSBs (de Jager et al., 2001), through the DNA-binding motif of MRE1. This binding is arranged as a globular domain with RAD50 WalkerA and B motifs and bridge DNA molecules.

MRN complex is thus the first sensor of DSBs and it activates ATM (Mirzoeva and Petrini, 2003; Lavin 2007) by two steps. First, it increases the local concentration of DNA ends to a level that triggers ATM monomerization. Then NBN binding to ATM converts it into active conformation (Dupre et al., 2006). Once activated, ATM plays the central role in DSB signalling and phosphorylates a variety of protein targets. For instance, ATM induces cell cycle arrest through the action of p53 intermediate (Canman et al., 1998; Waterman et al., 1998). Other substrates, e.g. NBS, MRE1, BRCA1, CHK2, FANCD2, Artemis and DNA-PKcs are phosphorylated by the activated ATM kinase and are important to determine the fate of the cells by play roles in DNA repair, cell cycle control, and transcription. The MRN complex and ATM are interdependent in the recognition and signalling of DSBs (Lavin, 2007).

A rapid phosphorylation of H2A by ATM at the C-terminal S139 residue is also observed in response to DSBs (Burma et al., 2001; Stiff et al., 2004). Phosphorylated H2AX (γH2AX) was found on megabase regions surrounding DSBs within seconds and function as a DNA damage signal transduction by serving as a docking site for several proteins (Kim et al., 2006).

The nuclease activity of MRE11 has been found to regulate the generation of single-stranded DNA in cooperation with CtIP in mammalian cells (Limbo et al., 2007; Sartori et al., 2007) by processing the 3′-ssDNA, a binding site for RPA (White and Haber, 19990). The RPA-ssDNA complex inhibits any further nuclease activity and provides the site of action of repair machinery (Sugiyma et al., 1997; Williams et al., 2007). This is followed either by HR or A-NHEJ, depending on the presence of homologous sequences, protein regulation and the size of resection (Rass et al., 2009).

CtIP was first characterized as a cofactor for the transcriptional repressor CtBP (carboxy-terminal binding protein) and for its binding to cell cycle regulators, such as the retinoblastoma protein and BRCA1 (Fusco et al., 1998; Schaeper et al., 1998; Wong et al., 1998). CtIP is known to have both transcription-dependent and independent implications in cell cycle progression (Liu and Lee, 2006; Wu and Lee, 2006). In addition to its central role in the cell cycle checkpoint response to DNA DSB, recent work suggested that CtIP controls the decision to repair DSB damage by HR by initiating DBS end resection (Sartori et al., 2007; You et al., 2009). In addition, it might also participate in the limited resection for DSB ends required for MMEJ during G1 phase (Yun and Hiom, 2009). Therefore, CtIP links cell cycle control, DNA damage checkpoints and repair. As the MRN complex is also necessary for DSB end resection, it is likely that CtIP provides a physical connection between the MRN complex and BRCA1 (Bernstein and Rothstein, 2009; Takeda et al., 2007).

Mutations in any of these genes involved in DNA repair, lead to genomic instability syndromes, such as ataxia-telangiectasia-like disorder (ATLD) (Steward et al., 1999; Taylor et al., 2004), Nijmegen breakage syndrome (NBS) and a variant form of Nijmegen breakage syndrome (Bendix-Waltes et al., 2005) for mutation in MRE11, NBS, and RAD50, respectively. In addition, null mutations lead to embryonic lethality in mice (Xiao and Weaver 1997; Luo et al., 1999; Zhu et al., 2001). Other mutations in the ATM or ATR genes cause genome instability syndromes, such as ataxia-telangiectasia (A-T) or Seckel syndrome (SCKL1), respectively (O'Driscoll et al., 2003). Artemis deficiency (Moshous et al., 2001), DNA ligase IV deficiency (LigIV) (O'Driscoll et al., 2001), Cernunnos-XLF (XRCC4-like factor) deficiency (Buck et al., 2006), Bloom syndrome (BS), Werner Syndrome (WS), and Fanconi anemia (FA) are associated with other members of the DNA damage repair machinery (Taniguchi and D'Andrea, 2006). Furthermore, in addition to genomic instability disorder, the patients with such syndromes often suffer from various types of malignancies, which indicate a link between unrepaired DNA damages and cancer occurrence. Genes involved in DNA repair play thus a critical role in tumor prevention.

As the person skilled in the art will readily understand any mutation in or around one of the genes of the above referenced that will enhance HR, decrease NHEJ and/or enhance the HR/NHEJ ratio, e.g., by shifting the choice between repair pathways towards HR, is within the scope of the present invention. Similarly, any protein or transgenic sequence acting on any one of the above pathways to enhance HR, decrease NHEJ and/or enhance the HR/NHEJ ratio is within the scope of the present invention.

### Enhanced Secretion of Transgene Expression Product IN Non-Primate Eukaryotic Host Cells

In transfected cell populations there are generally a small minority of cells that produce considerable amounts of the transgene expression product (medium or high producer clones/cells displaying more than 10-100 and 100-1000, respectively relative light units (RLUs)) and cells that hardly produce any transgene expression product (low producer clones/cells, e.g., displaying less than 10 RLUs). However, in some cases, no high producer clones/cells can be obtained from specific transgenes. It could be shown that this differences is transgene expression is product specific and that there are certain “difficult-to-express” proteins. Low producer cells for such difficult-to-express proteins, but to a small extent also medium and high producer cells, e.g., for easy-to-express proteins, showed intracellular precipitation of in particular precursor protein and potentially polypeptide cross-linking, thus indicating possible problems in the processing, folding and/or assembly of the final product.

Further data presented herein suggested problems in protein secretion, in particular in ER translocation and processing. Despite previous unsuccessful attempts to increase protein secretion by expressing components of the protein secretion pathway (Lakkaraju et al., 2008), the combination of a non-primate cell, such as a CHO cell and transgenic sequence, in particular a primate, e.g. human sequence encoding such a component resulted in the surprising improvement of secretion of not only the low producer cells, but also in high producer cells. Of the components tested, particular ones and particular combinations entailed particularly favorable results. For example, SRP14 was one of the proteins that was successfully tested. It may be required to halt elongation until the nascent polypeptide may find an available SR with the help of SRP54. Since a further combination with Translocon (Transl) provided particularly high secretion, the resulting complex may need to associate to Transl in order for translocation to occur, which itself leads to the removal of the signal peptide in the endoplasmic reticulum and to the secretion of a properly processed and assembled protein. However, as the person skilled in the art will readily understand, the introduction of transgenic sequences of other components and the combinations of such sequences is within the scope of the present invention. Reference is in particular made to the description of the protein secretion pathway elsewhere herein.

Herein, the term translocation is primarily used to refer to the transport across the membrane of the endoplasmic reticulum. It should, however, be recognized that the term is often used in the literature to refer to a more generic concept.

The Protein Secretion Pathway

The secretion of proteins is a process common to organisms of all three kingdoms. This complex secretion pathway requires most notably the protein translocation from the cytosol across the cytoplasmic membrane of the cell. Multiple steps and a variety of factors are required to for the protein to reach its final destination. In mammalian cells, this secretion pathway involves two major macromolecular assemblies, the signal recognition particle (SRP) and the secretory complex (Sec-complex or translocon). The SRP is composed of six proteins with masses of 9, 14, 19, 54, 68 and 72 kDa and a 7S RNA (Keenan, Freymann et al. 2001) and the translocon is a donut shaped particle composed of Sec61αβγ, Sec62 and Sec63.

The first step in protein secretion depends on the signal peptides, which comprises a specific peptide sequence at the amino-terminus of the polypeptide that mediates translocation of nascent protein across the membrane and into the lumen of the endoplasmic reticulum (ER). During this step, the signal peptide that emerges from the leading translating ribosome interacts with the subunit of the SRP particle that recognizes the signal peptide, namely, SRP54. The SRP binding to the signal peptide blocks further elongation of the nascent polypeptide resulting in translation arrest. The SRP9 and −14 proteins are required for the elongation arrest (Walter and Blobel 1981). In a second step, the ribosome-nascent polypeptide-SRP complex is docked to the ER membrane through interaction of SRP54 with the SRP receptor (SR) (Gilmore, Blobel et al. 1982; Pool, Stumm et al. 2002). The SR is a heterodimeric complex containing two proteins, SRα and SRβ that exhibit GTPase activity (Gilmore, Walter et al. 1982). The interaction of SR with SRP54 depends on the binding of GTP (Connolly, Rapiejko et al. 1991). The SR coordinates the release of SRP from the ribosome-nascent polypeptide complex and the association of the exit site of the ribosome with the Sec61 complex (translocon). The growing nascent polypeptide enters the ER through the translocon channel and translation resumes at its normal speed. The ribosome stays bound on the cytoplasmic face of the translocon until translation is completed. In addition to ribosomes, translocons are closely associated with ribophorin on the cytoplasmic face and with chaperones, such as calreticulin and calnexin, and protein disulfide isomerases (PDI) and oligosaccharyl transferase on the luminal face. After extrusion of the growing nascent polypeptide into the lumen of the ER, the signal peptide is cleaved from the pre-protein by an enzyme called a signal peptidase, thereby releasing the mature protein into the ER. Following post-translational modification, correct folding and multimerization, proteins leave the ER and migrate to the Golgi apparatus and then to secretory vesicles. Fusion of the secretory vesicles with the plasma membrane releases the content of the vesicles in the extracellular environment.

Remarkably, secreted proteins have evolved with particular signal sequences that are well suited for their own translocation across the cell membrane. The various sequences found as distinct signal peptides might interact in unique ways with the secretion apparatus. Signal sequences are predominantly hydrophobic in nature, a feature which may be involved in directing the nascent peptide to the secretory proteins. In addition to a hydrophobic stretch of amino acids, a number of common sequence features are shared by the majority of mammalian secretion signals. Different signal peptides vary in the efficiency with which they direct secretion of heterologous proteins, but several secretion signal peptides (i.e. those of interleukin-, immunoglobulin-, histocompatibility receptor-signal sequence, etc) have been identified which may be used to direct the secretion of heterologous recombinant proteins. Despite similarities, these sequences are not optimal for promoting efficient secretion of some proteins that are difficult to express, because the native signal peptide may not function correctly out of the native context, or because of differences linked to the host cell or to the secretion process. The choice of an appropriate signal sequence for the efficient secretion of a heterologous protein may be further complicated by the interaction of sequences within the cleaved signal peptide with other parts of the mature protein (Johansson, Nilsson et al. 1993).

### DETAILED DESCRIPTION OF THE FIGURES

### Effect of Repeated Transfection on Transgene Expression

Certain human MARs, e.g., MAR 1-68, have been found to potently increase and stabilize gene expression in cultured cells as well as mice when inserted upstream of the promoter/enhancer sequences (Girod et al. 2007, Galbete et al. 2009).

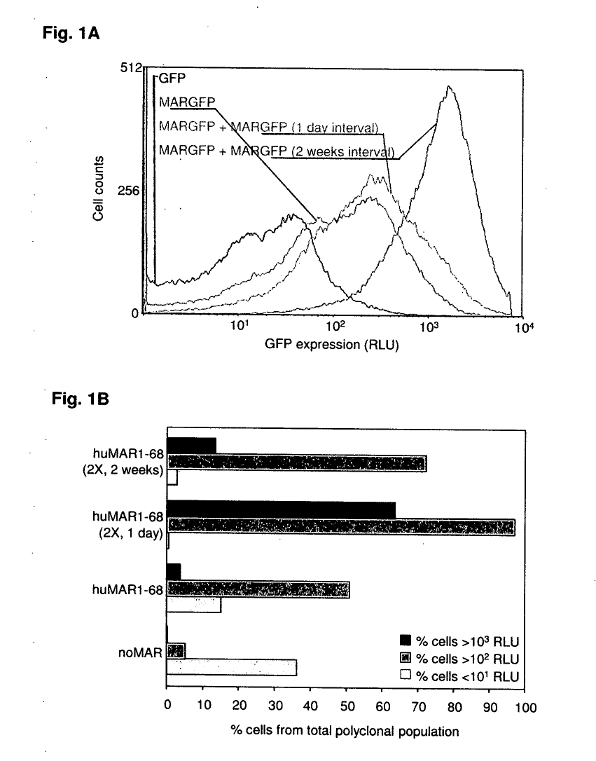

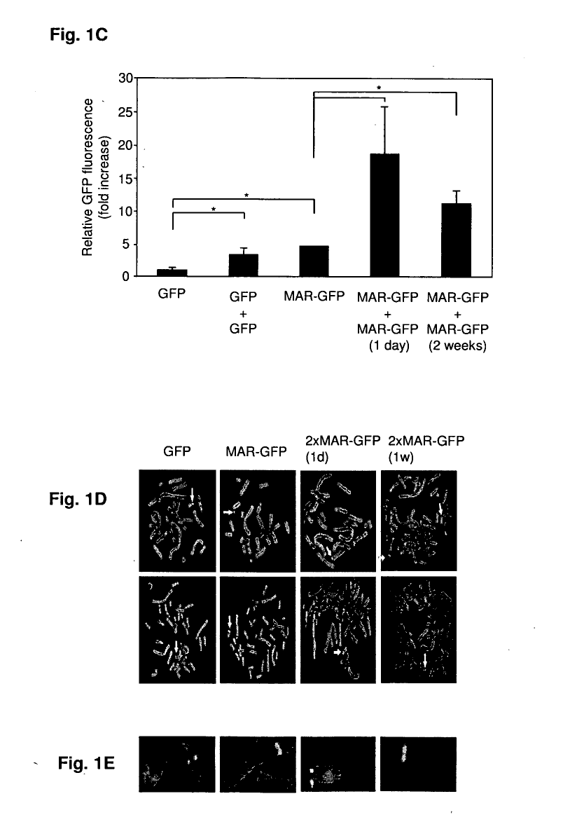

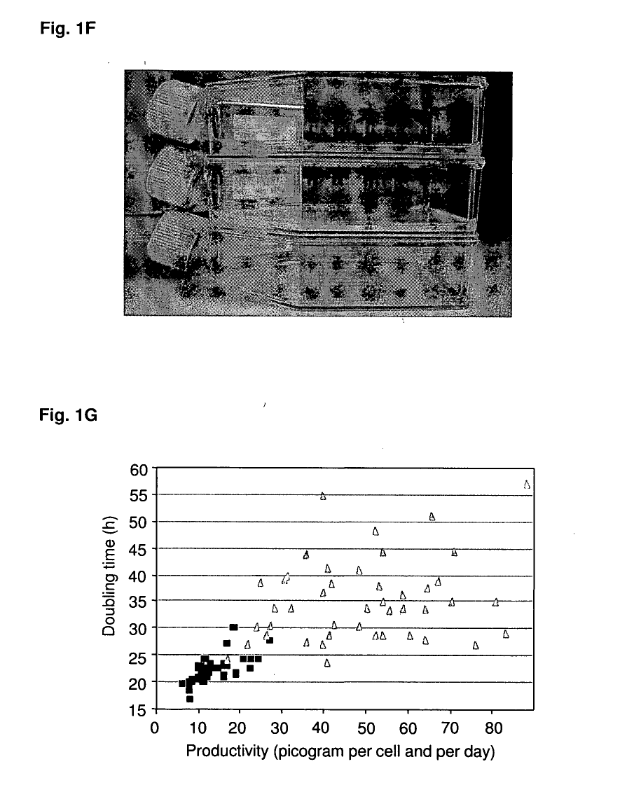

An analysis of the effect of MARs and successive transfections on gene transfer and expression is shown in FIG. 1. FIG. 1(A) depicts the fluorescence distribution in polyclonal populations of GFP-expressing cells. CHO DG44 cells were co-transfected with the GFP expression vector devoid of MAR element (GFP, left profile), or with the vector containing MAR 1-68 (MAR1-68GFP, second from left profile), and with the pSVpuro plasmid mediating resistance to puromycin. Some of these cells were subjected to a second transfection with the same GFP expression vector but with a selection plasmid mediating neomycin resistance, either on the day following the first transfection (right profile) or after 2 weeks of selection for puromycin resistance (second from right profile). After two weeks of selection for puromycin and/or neomycin resistance, eGFP fluorescence was quantified by cytofluorometry. The profiles shown are representative of four independent experiments. In FIG. 1(B), a histogram shows the percentage of total cells corresponding to non/low-expressors that display less then 10 relative light units (RLU), or cells that display medium and high (>100 RLU) or very-high (>1000 RLU) GFP fluorescence, as determined from the analysis of stable cell pools as shown in panel A. In FIG. 1(C) the mean GFP fluorescence of each stable polyclonal cell pool was normalized to that obtained from the transfection of MARGFP and the average and standard deviation of four independent transfections is shown as a fold increase over the fluorescence obtained by one transfection without a MAR. Asterisks indicate significant differences in GFP expression (Student's t-test, P<0.05). FIG. 1(D) depicts the results of a FISH analysis of eGFP transgene chromosomal integration sites in cells singly or doubly transfected with or without the human MAR. Metaphase chromosomes spreads of stable cell pools were hybridized with the GFP plasmid without MAR, and representative illustrations of the results are shown. In FIG. 1(E), enlargements of chromosomes are shown to illustrate differences in fluorescence intensities.

The Figures show that co-transfection of a GFP expression vector and an antibiotic resistance plasmid, followed by antibiotic selection of cells having stably integrated the transgenes in their genome, typically yields a bimodal distribution of the fluorescence in polyclonal cell populations when analyzed by flow cytometry (FIG. 1A). A first cell subpopulation, which overlaps the Y-axis in this experimental setting, corresponded to cells expressing GFP at undetectable levels, while another subpopulation of cells express significant GFP levels. Inclusion of MAR 1-68 increased the level of expression from fluorescent cells and concomitantly reduced the proportion of silent cells (15% vs 36%, FIG. 1B).

When the same GFP expression vector was co-transfected two weeks later with a distinct antibiotic resistance gene, a 2.4-fold increase of fluorescence was observed on average after selection for resistance to the second antibiotic, which is close to the expected 2-fold increase (FIGS. 1A and 1C). In contrast, an unexpectedly higher (4 to 5 fold) increase of GFP expression was observed from two successive transfections performed on consecutive days followed by selection with both antibiotics. On average, over all cells of the polyclonal population, a 20-fold increase of expression was obtained by successive transfections of MAR-containing plasmids relative to a single transfection without a MAR (FIG. 1C). Furthermore, some of the cells displayed very high levels of expression, and the occurrence of silent cells was almost fully abrogated from the polyclonal population (0.5%, FIG. 1B). Consecutive transfections without a MAR yielded modest GFP expression, resulting in a 3.2-fold increase of the overall fluorescence level when compared to a single transfection, and it did not abrogate the occurrence of silent cells (FIG. 1C and data not shown). Thus, the presence of the MAR and the repeated transfection act synergistically to mediate elevated expression levels.

Overall, the expression levels obtained from the two consecutive (a first and a subsequent) transfections of MAR-containing plasmids were so high that the GFP fluorescence could be readily seen from the cell culture monolayers in the daylight, without UV irradiation (FIG. 1F, which shows that levels of GFP expression obtained from stable polyclonal cell pools transfected twice with MAR-GFP vector by the visible GFP fluorescence of cell monolayers in day light). This effect was not limited to the GFP transgene or to the SV40 promoter used in this study, as similar results were obtained with plasmids carrying a CMV promoter and a DsRed reporter gene, and very high expression of a therapeutic immunoglobulin was also obtained upon successive transfections (Data not shown and FIG. 1G, which shows the relationship between transgene expression and the duration of the cell division cycle. Immunoglobulin G expression vectors containing human MAR 1-68 upstream of the SV 40 promoter and each of the light and heavy chain were co-transfected with an antibiotic resistance plasmid, and antibiotic-resistant cells were selected for three weeks. Monoclonal cell populations were isolated by two rounds of limiting dilutions and the amount of secreted IgG and average cell division time were determined. Squares and triangles illustrate clones obtained after one or two transfections, respectively). Interestingly, the very high levels of immunoglobulins expressed by monoclonal CHO cell clones often correlated with an increased cell division time. This indicates that the cells were likely reaching their physiological limits in terms of protein synthesis. This may be expected, as cells were synthesizing similar amounts of the recombinant protein when compared to their own cellular proteins (approximately 100 pg per cell). This should double the energetic input required at each cell division. Nevertheless, a large proportion of clones were found to express the heterologous protein at very high levels without interfering with their own metabolism, as they did not slow down cell division significantly (FIG. 1G).

### Cointegration of Transgenes Upon Repeated Transfections

An important parameter driving high expression upon repeated transfection was found to be the time interval between the transfections. The synergistic effect on expression was not observed when re-transfecting cells after two weeks, when the two transfections behaved as two independent and thus additive events (FIG. 1C). This suggests that the DNAs of each transfection may have to interact as extrachromal episomes within the nucleus and may form mixed concatemers before integrating into the cell genome. This was assessed by fluorescent in situ hybridization (FISH) analysis of metaphase chromosomal spreads from stable polyclonal populations. 80 individual metaphases of cells transfected once either with or without the MAR element were hybridized with a probe consisting of the GFP plasmid without a MAR. A single integration site was observed, but higher fluorescence intensities were observed from cells transfected with the MAR (FIGS. 1D and E). Fluorescence intensity was further increased by the double transfection process, suggesting that a higher number of transgene copies had integrated. Unique integration sites were noted in all cases after a single or two consecutive transfections. However, double integration events were observed in approximately half of the cells transfected twice at an interval of one week, when little extrachromosomal episomal DNA should remain from the first transfection. This indicates that independent integration events may occur if DNA integration from the first transfection has been completed before the second transfection is performed. Double transfections did not lead to detectable chromosomal rearrangements, nor did they detectably lead to insertions at a preferred chromosomal locus, as none of the analyzed cells had an identical integration site. Thus, transgenes integration upon two transfections does not appear to be targeted to any specific chromosomes or chromosomal sites, as reported earlier for single transfections of MAR-containing plasmids (Girod et al. 2007).

### High Transgene Expression and Phasing of the Cell Cycle and Transfections

As timing between transfections seemed to play a role in high transgene expression, the effect of systematic variations of the time interval between transfections was analyzed. In the model cells, the highest GFP expression level was observed when the second transfection was performed 21 hours after the first one, yielding consistently a five-fold increase of fluorescence as compared to a single transfection. FIG. 2 depicts how the optimal timing between successive transfections was determined. FIG. 2(A) shows that stable polyclonal populations were generated by a single transfection (minus sign) or by two consecutive transfections of the MAR-GFP expression plasmid separated by the indicated time intervals. After two weeks of selection, mean GFP expression of the total polyclonal populations was determined. Fluorescence levels were normalized to the maximal values obtained and are displayed as the fold increase over the expression obtained from a single transfection wherein (n) corresponds to the number of independent transfections. Asterisks indicate significant differences in GFP expression (Student's t-test, P<0.05). FIG. 2(B) shows an analysis of the cell cycle progression. At the time of first and second transfections, CHO cells were harvested and stained with propidium iodide and fluorescence was analyzed by cytofluorometry. The distribution of relative propidium iodide (PI) fluorescence represents the amount of genomic DNA per cell. The percentage of the population associated to each cell cycle state (G1, S, G2/M) is as indicated. The results show that when the second transfection was performed after 18 h, 24 h and 27 h, a 3 to 3.5-fold increase of expression was obtained relative to a single transfection. However, this increase was significantly lower than that obtained after 21 h (FIG. 2A). As this period is close to the duration of the cell division cycle after cell passaging (FIGS. 2 C and D), this suggests that high transgene expression upon consecutive transfections might be linked to particular phases of the cell division cycle.

The distribution of the cells along the division cycle was determined by propidium iodide staining of the DNA. This analysis indicated an over-representation of cells at the G1 phase 18 h after cell passaging, and this was found to correspond to the timing that yields the highest expression from a single transfection (FIG. 2B, data not shown and FIGS. 2 C and D, which show the cell culture progression through the cell division cycle. FIG. 2(C) represents time profiles for cell cycle progression. CHO DG44 cells were harvested for cell cycle analysis every two hours, starting at 18 hours after cell passage, which corresponds to the optimal timing for the first transfection. Cells were fixed and DNA was stained with propidium iodide before acquisition of the fluorescence level of 10,000 cells. FIG. 2(D) shows the determination of the cell cycle duration. The percentage of cells in G1 phase was determined every two hours after passaging the cells. The bracket indicates timing between two maxima, which was taken as one cycle duration (14 hours). The extended first cycle of 18 h is perturbed because of cell passaging at t=0, and the delay is attributed to the 4 additional hours required for the cells to adhere to the culture dish surface and to resume cell division cycle progression). A similar pattern and over-representation of G1 cells was obtained 21 h after the first transfection, which again corresponds to the timing that yields the highest expression levels upon a second transfection. If expression is indeed linked to cell cycle phasing, another optimum for transgene expression should be observed when the second transfection is performed at an interval corresponding to two cell divisions. After 42 hours, the synergistic effect of the two transfections was lost, as expression was similar to that obtained for one transfection. However, a second, albeit lower, synergistic increase of transgene expression was observed after 48 hours. The higher levels of expression observed from a first transfection at 18 h and for a second transfection performed with a 21 or 48 h interval imply that optimal DNA transfer and/or expression may occur at specific cell division stages.

### Effect of MAR and Consecutive Transfections on Cellular DNA Uptake

FISH analysis suggested that elevated expression upon successive transfections may result in part from the integration of a higher number of the transgene copies in the genome (FIG. 1D). Consecutive transfections at an interval of one day might lead to an increase of the concentration of plasmid episomes in the nucleus, thus augmenting the probability of transgene integration within the cell genome. To assess the amount of transgene entering nuclei at each transfection, a transient single and double transfections, respectively was/were performed followed by plasmid extraction from nuclei isolated 1 or 2 days after the second transfection and quantification of the transgenes by real-time quantitative PCR (qPCR).

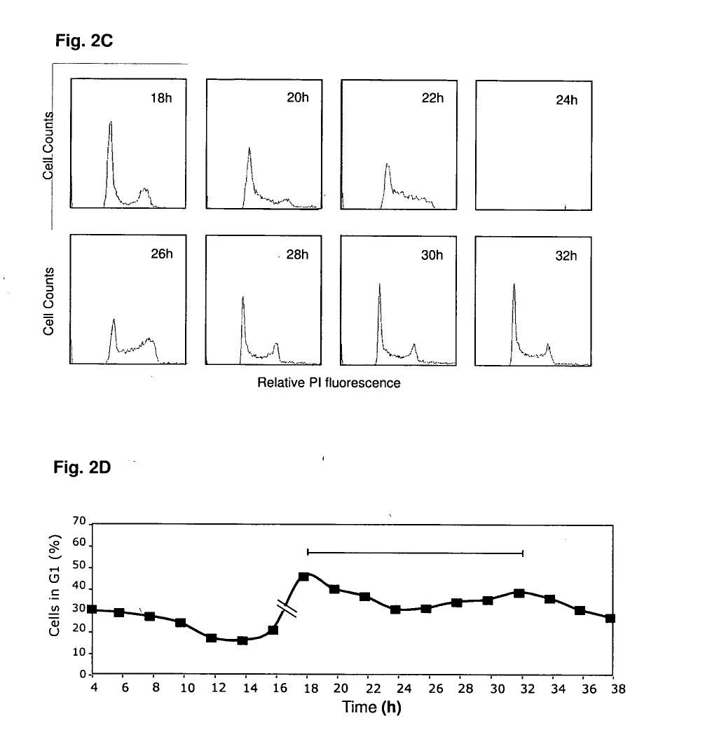

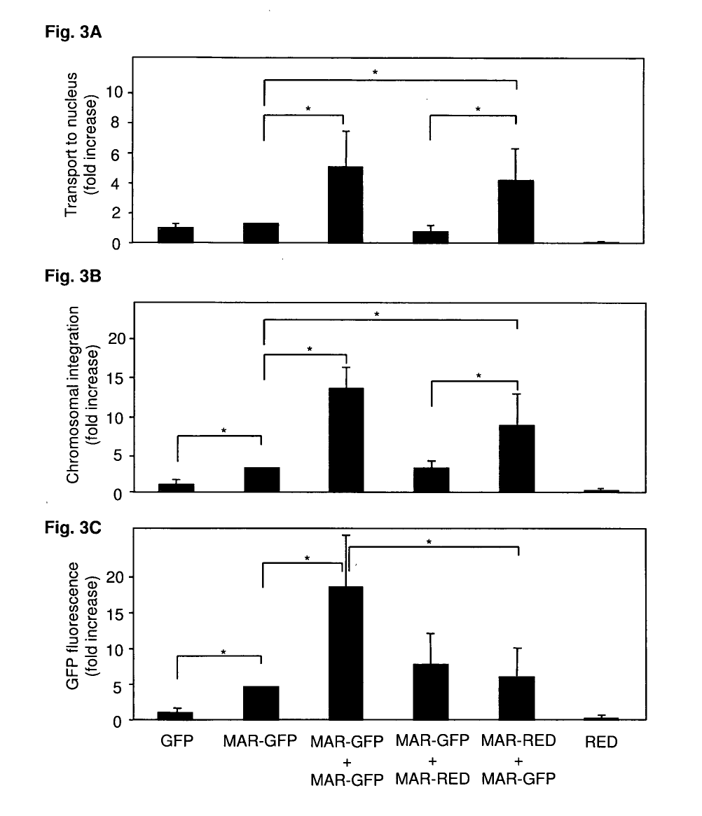

FIG. 3 shows DNA transport, integration and expression upon successive transfections. FIG. 3(A) shows the amount of GFP transgenes transport into cell nuclei during single and double transient transfections with GFP or DsRed (“RED”) plasmids with or without a MAR. MAR-GFP+MAR-RED corresponds to a double transfection where MAR-GFP is transferred during the 1^{st} transfection, whereas MAR-RED was used in the second transfection. Nuclei were isolated and total DNA was extracted one day after a single or after the 2^{nd} transfection, respectively, and the number of GFP transgenes transported into the nuclei was quantified by qPCR. Results were normalized to that of the reference CHO cell genomic GAPDH gene and represent the mean of 4 independent transfections. FIG. 3(B) shows the effect of the MAR and successive transfections on integrated GFP transgene copy number. Total genome-integrated transgene DNA was extracted from the previously described GFP-expressing cells after 3 weeks of selection of stable polyclonal cell pools, and DNA was quantified as for A. FIG. 3(C) shows the effect of MAR and successive transfections on GFP expression. The GFP fluorescence levels of the stable cell pools analyzed in B were assayed by cytofluorometry.

FIGS. 3 (D) and (E) show the relationship between mean GFP fluorescence and transgene copy number in monoclonal cell populations.

In FIG. 3(D), the mean GFP fluorescence levels of distinct stable cell clones transfected with GFP, MAR-GFP or transfected twice with MAR-GFP are presented as a function of their transgene copy number per genome, as determined by qPCR.

In FIG. 3(E), the relationship between GFP fluorescence levels normalized to the transgene copy number is represented as a function of the number of integrated transgene copies per genome. The calculated regression curves are represented by dashed line and R² indicates the correlation coefficient.

The results show that cells doubly transfected with MAR-GFP exhibited 3.8-fold more GFP transgene copies in their nuclei than cells transfected just once with MAR-GFP (FIG. 3A). When comparing cells transfected with these different plasmids expressing either GFP or DsRed (“RED”), it was observed that the nuclear delivery resulting from the second transfection of MAR-GFP was 4.2-fold higher than the one observed from a single transfection of this plasmid. However, the nuclear transport of the firstly transfected GFP plasmid was not increased significantly by performing a second transfection. It was concluded that DNA transport to the nucleus from the second transfection is favored by performing a prior first transfection.

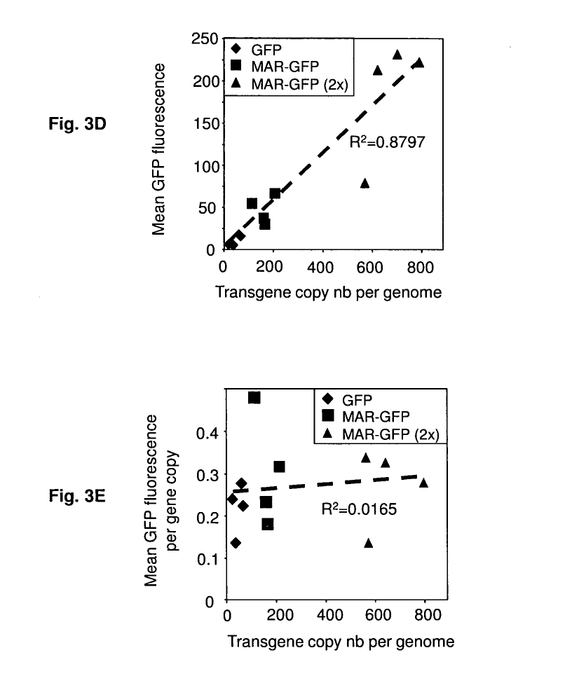

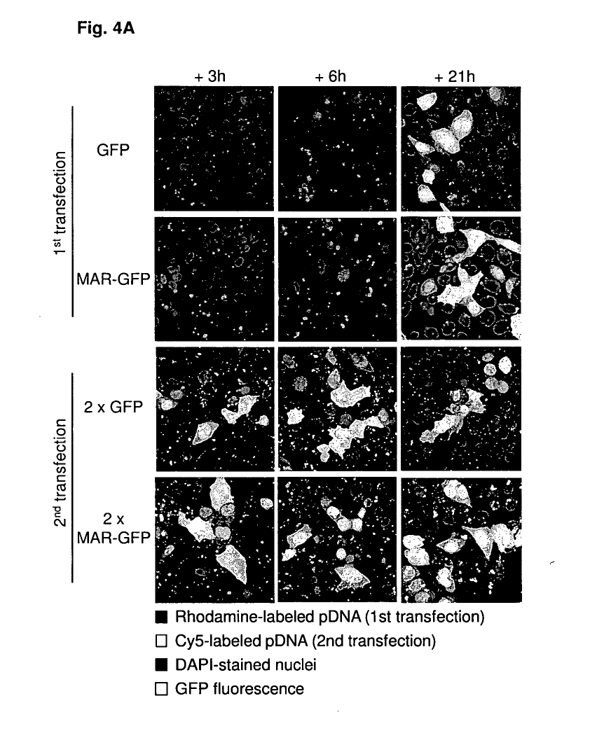

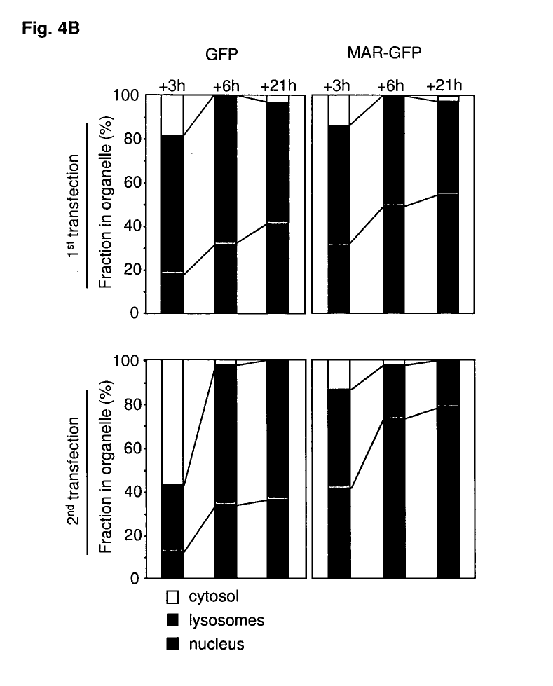

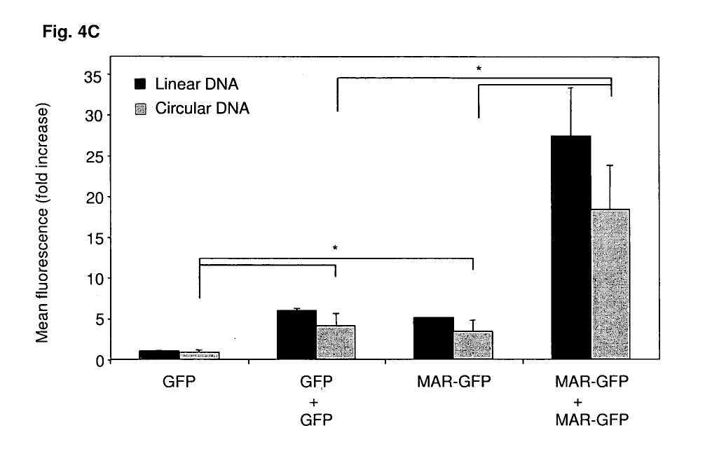

These conclusions were strengthened by confocal imaging of DNA transport, where plasmids used for the first transfection were labeled with rhodamine while the secondly transfected plasmids were labeled with Cy5 (dark (small dots) and white (small dots) labels respectively, FIG. 4A). In particular, FIG. 4 depicts the subcellular distribution of transfected DNA. FIG. 4(A) shows a confocal microscopy analysis of DNA intracellular trafficking. Transient single or double transfections were performed in CHO cells using plasmids bearing or not a MAR labeled with Rhodamine and Cy5 fluorophores, as indicated. Transfected cells were fixed and stained with DAPI (large dark areal spots) 3 h, 6 h, 21 h post-transfections. Cells expressing GFP appear as large light areal spots on the pictures. FIG. 4(B) shows the quantification of the subcellular plasmid DNA distribution, which was performed on confocal laser microscopy performed for A, except that endosome/lysosome compartments were stained with LysoTracker Red DND-99. The pixel area of clusters derived from rhodamine or Cy5 fluorescence were used to estimate the amount of plasmid DNA in approximately 120 cells. Similar numbers of rhodamine-labeled plasmid clusters were observed in cell nuclei after a first transfection with or without a MAR, which correlates well with the lack of effect of the MAR on DNA transport as assessed by qPCR (FIGS. 3A and 4A). Nuclear plasmid clusters were observed in essentially all the cells after two transfections. However only few cells expressed GFP, in agreement with previous observations that only a minority of cells are able to express transiently transfected genes (Akita et al. 2007).

The transport of transfected plasmid DNA in CHO cells, which is known to comprise cellular uptake, lysosomal escape and nuclear import, is limited by endosomal/lysosomal degradation (Akita et al. 2007). Thus, the intracellular trafficking of transfected plasmid DNA was assessed by quantifying its distribution in cellular organelles and in the cytosol after each transfection, after specific staining of the endosomal/lysosomal and nuclear compartments to distinguish them from the cytosol. Results summarized in FIG. 4B shows a similar subcellular distribution of plasmid DNA with or without MAR21 h after a first transfection, although nuclear transport of MAR-containing plasmids seemed somewhat faster at the earlier time points. Performing a second transfection of the MAR-devoid plasmid did not yield an improved nuclear transport. However, plasmids bearing a MAR element escaped lysosomal retention and entered nuclei much more efficiently, as 80% of the total Cy5-labeled pDNA was located in the nuclei in presence of the MAR 21 h after the second transfection, as compared to less than 40% of the plasmid devoid of MAR. Rather, most of the MAR-devoid plasmid ended up in the lysosomal/endosomal compartment, as found also for the first transfection. The unexpected finding of a cooperative effect of the MAR and of repeated gene transfer on lysosomal escape thus provides an explanation for the increased concentration of episomes in isolated nuclei (FIGS. 3A and 4B). This phenomenon might in part result from the saturation of the cellular degradation compartments by the DNA of first transfection, thus allowing plasmids of the second transfection to remain in the cytoplasm where the MAR may promote transport into the nucleus.

### MAR Elements Increase the Copy Number of Genome-Integrated Transgenes

Next, it was tested whether the increased transport of plasmid DNA elicited by the MAR and the consecutive transfections may increase transgene integration into the genome of CHO cells. Stable polyclonal cell populations were selected as for FIG. 1, and the average numbers of stably integrated GFP transgene copies per genome were determined on total cell DNA using qPCR. Inclusion of a MAR element in transfected plasmids significantly increased the number of transgenes integrating in the genome of stable cell pools (FIG. 3B). As the MAR does not act to increase nuclear transport after single transfections (FIG. 3A), this implied that the MAR can increase genomic integration of the plasmid per se. This finding substantiates previous suggestions that the use of MARs increases the number of transgene copies that integrate in the genome of recipient cells (Girod et al. 2005, Kim et al. 2004).

Successive transfections also mediated a 4-fold increase of plasmid integration, which is commensurate to the increase in free extracellular episomes noted in transient transfections (FIGS. 3A and 3B). It could be estimated that 48 GFP plasmid copies had integrated on average when transfecting once without a MAR, while approximately 163 copies and 676 copies on average were obtained from one or two successive transfections with the MAR, respectively. Overall, the increased nuclear transport synergistically elicited by both the MAR and the successive transfections yielded a more than 10-fold increase in transgene copy number when combined to the MAR-driven increase of plasmid integration. This yielded yet an even higher increase in transgene expression (over 15-fold, FIG. 3C), as expected from the previously observed antisilencing effect of the MAR (Galbete et al. 2009).

When assessing GFP expression and transgene copy number in individual cell clones isolated from the polyclonal populations, a good overall correlation was found between transgene expression and copy number (FIG. 3D). This indicates that the transgene copy number is the main driver of the increased expression upon the double transfection of MAR-containing plasmids. Furthermore, no significant decrease of expression could be detected from MAR-containing clones having co-integrated very high numbers of transgene copies and MARs (FIG. 3E). Thus, it was concluded that the MAR was able to prevent inhibitory effects that may result from the repetitive nature of the co-integrated plasmids and/or from antisens transcription, an effect that can be attributed to the potent anti-silencing properties of this MAR element (Galbete et al. 2009). However, the average levels of expression did not always match perfectly the copy number, as noted when analyzing individual cell clones, or when comparing GFP expression from the firstly or secondly transfected DNA, in co-transfection experiments with the dsRED vector (FIGS. 3B and 3C). This led to the conclusion that the enhanced transgene expression observed after two transfections of MAR-containing plasmids can be explained in part by the improved nuclear import and genomic integration and hence transgene copy number, the lack of silencing, as well as by a higher transgene expression per transgene copy, but that other effects may also influence transgene expression depending on the transfection history and conditions.

### Effects of DNA Homology on Plasmid Integration and Expression

As the high GFP fluorescence observed from successive transfections of MAR-containing plasmids results in part from the increased transgene integration at a single chromosomal locus, the molecular basis of this effect was assessed. For instance, the integration of a MAR-containing plasmid during the first transfection might promote secondary integration at the same genomic locus during the second transfection, as could be expected from the ability of the MAR to maintain chromatin in an accessible state and thus to provide proper targets for homologous recombination. Alternatively, the high number of integrated transgene copies observed from successive transfections may result from a more efficient concatemerization of the plasmids introduced during both transfections, as may be mediated by the high concentration of extrachromosomal episomes in the nucleus. Homologous recombination was proposed to mediate the formation of large concatemers of transfected plasmids (Folger et al. 1985), which may lead to the co-integration of multiple plasmid copies upon recombination with the genomic DNA. In the latter model, homologous recombination may occur between similar plasmid sequences on the plasmids used during the first and second transfections, and thus the efficacy of transgene integration and expression should depend on DNA sequence homologies.

This latter possibility was first assessed by analyzing the effect of plasmid homology on transgene expression by performing successive transfections with different combinations of transgenes (GFP or DsRed), plasmid backbones (ampicillin or kanamycin bacterial resistance) and/or MARs (chicken lysozyme MAR or the human MAR 1-68).

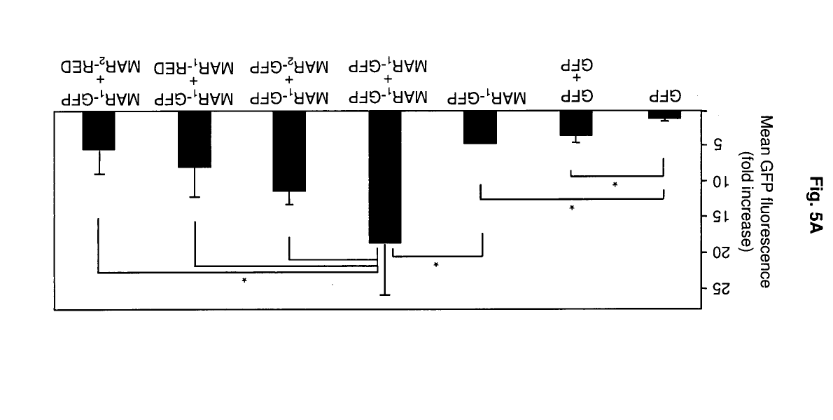

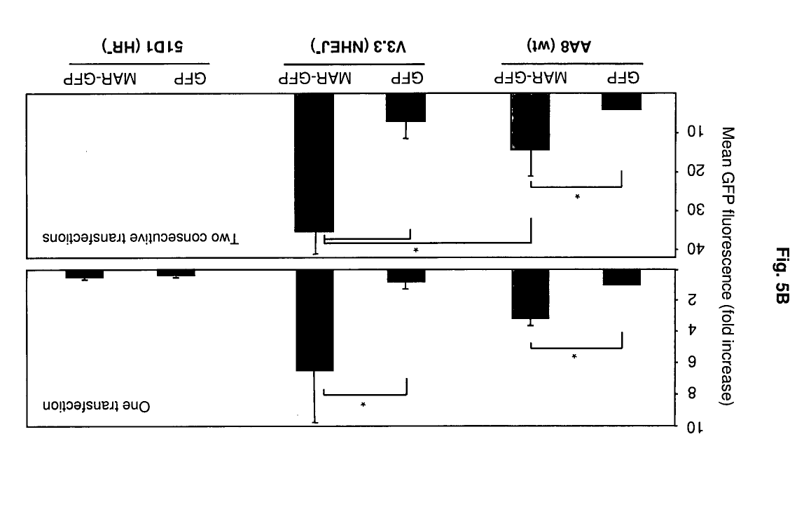

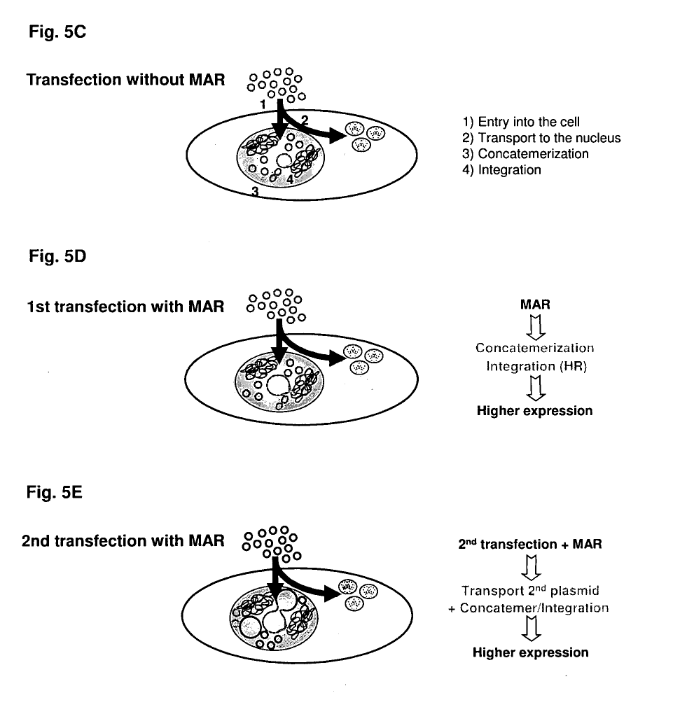

FIG. 5 shows how the MAR, plasmid homology and homologous recombination mediate high transgene expression. For FIG. 5(A) stable polyclonal cell pools were generated by the transfection of plasmids bearing different transgenes (GFP or DsRed (“RED”)), MAR (MAR₁ for the human 1-68 or MAR₂ for the chicken lysozyme MAR), and/or bacterial resistance gene (ampicillin or kanamycin), and the relative average GFP fluorescence of four independent transfections are shown as the fold increase over that obtained from one transfection without MAR. Asterisks show significant differences in GFP expression (Student's t-test, P<0.05). For FIG. 5(B) stable transfections with GFP or MAR1-68GFP plasmids were performed in the parental CHO cell line (AA8) and in mutants deficient either in the homologous recombination (51D1) or non-homologous end-joining (V3.3) pathway. The mean GFP fluorescence of each stable polyclonal cell pool generated from single (top panel of (B)) or two consecutive (bottom panel of (B)) transfections were normalized to that obtained from AA8 cells singly transfected with the MAR-devoid plasmid. Asterisks indicate significant differences in GFP expression (Student's t-test, P<0.05). No stably transfected cells were obtained from the double transfection of 51D1 cells.

The results show that transfection of distinct MARs, transgenes, or bacterial resistance all decreased the high expression normally observed with successive transfections (FIG. 5A). The double transfection effect was almost fully abolished when using different MARs, transgenes and vector elements (MAR₁-GFP+MAR₂-RED constructs), suggesting that plasmid homology is required to achieve high expression from successive transfections.

Homologous recombination is often elicited as a DNA repair mechanism of double-stranded breaks, in a process that was termed Homologous Recombination Repair (HRR, ADD REF). Thus, it was tested whether plasmid linearization prior to transfection mediates the high expression obtained from successive transfections.

FIG. 4(C) shows the effect of DNA conformation on gene transfer and expression. In order to compare transgene expression level after a single or successive transfections with linear or circular plasmids, the same equimolar amount of GFP and MAR-GFP circular DNA or Pvul-digested plasmids were used for transfection. After two weeks of selection, eGFP fluorescence of stably transfected cell populations was analyzed by cytofluorometry. The profiles display the GFP fluorescence level fold increase over that of control cells transfected once with the MAR-devoid plasmid. Fluorescence values obtained with linear or circular plasmids are presented in dark or light grey, respectively. Asterisks indicate some of the significant differences in GFP expression (Student's t-test, P<0.05).

A more than additive increase of transgene expression was also observed with circular plasmids, however, the overall expression was lower than that obtained using linear plasmids (FIG. 4C), consistently with the increased recombinogenic properties of linear DNA in homologous recombination processes (Wong et al. 1986).

### Homologous Recombination Mediates Increased Expression

The requirement of plasmid homology and double-strand breaks to achieve the higher expression levels upon the double transfection implies that homologous recombination may be involved. Transgenes were proposed to integrate into the cell genome using two families of antagonistic pathways, termed non-homologous end-joining (NHEJ) or homologous recombination (HR). These pathways are more active during specific phases of the cell cycle, as exemplified by HR, which is used to repair DNA damages during or after DNA replication in the S and G2/M phases of the cell cycle (Takata et al. 1998). Cells lacking classical NHEJ genes show a double-stranded break (DSB) repair biased in favour of HR, suggesting that these two major pathways normally compete to repair these DNA lesions (Delacote et al. 2002). Thus, one way to activate HR is to suppress or genetically inactivate NHEJ, as seen in yeast and mammalian cells (Delacote et al. 2002, Clikeman et al. 2001, Allen et al. 2002, Pierce et al. 2001). A possible implication of HR-related mechanisms in the increased transgene expression that results from the MAR and/or successive transfections was thus directly assessed using CHO cell lines mutated in a key component of either pathways, and which are thus only competent for either HR or NHEJ.

The 51D1 CHO mutant derivative lacks the RAD51 strand transferase and is thus deficient in homologous recombination, while V3.3 CHO cells lack the catalytic activity of DNA-dependent protein kinase DNA-PK that plays an essential role to initiate the NHEJ pathway (Jackson 1997, Hinz et al. 2006, Jeggo 1997). A 3-fold increase of the overall GFP fluorescence was mediated by the MAR in a polyclonal population of wild-type parental cell lines (AA8), as compared to cells stably transfected without the MAR (FIG. 5B). However, few stably transfected colonies survived after selection for antibiotic resistance in the 51D1 cell line and GFP expression remained very low. In contrast, an exacerbated MAR-driven activation of transgene expression was observed in NHEJ-deficient cells, resulting in a more than 6-fold increase of transgene expression when compared to cells transfected once with the GFP expression vector without MAR.

Similar trends were noted for successive transfections, in that GFP expression from V3.3 cells was increased both by the presence of the MAR and by the successive transfections as compared to parental AA8 cells (FIG. 5B, note the different scales of the top and bottom panels). Overall, a 35-fold increase in transgene expression was obtained from two consecutive transfections of NHEJ-deficient V3.3 cells with the MAR when compared to a single transfection of the control plasmid in parental cells. In contrast, inactivation of the NHEJ pathway had little effect on the expression of the MAR-devoid plasmid, indicating that the presence of the MAR and high HR activity are both necessary to obtain very high transgene expression. Consistently, cells deficient in HR yielded low expression levels a smaller effect of the MAR in single transfections, while no antibiotic-resistant colonies were obtained from the double transfection.

These results clarify the significance of the HR pathway in the integration and maintenance of the two selections genes used in the successive gene transfer process. FIGS. 5(C) to (E) show a model for improved expression by repeated transfection with MAR.

As can be seen in the scheme shown in FIGS. 5(C) and (D), the exponential increase of transgene expression is partly explained by an increased entry and genomic integration of plasmids into the cell nuclei, resulting both from the MAR element and from the double transfection process. After the first transfection, the presence of the MAR may augment the frequency of homologous recombination between transfected plasmids, allowing the formation of bigger concatemeric structure and integration of more plasmid copies. In addition, MAR may recruit proteins to remodel chromatin structure towards an open state. As can be seen in FIG. 5(E), plasmids of the second transfection may be more efficiently transported to the nucleus, as a consequence of the first transfection and of the possible saturation of the degradation compartments of the cells. The MAR elements may act to promote recombination as before, allowing a better concaterimerization of homologous plasmids from both transfections. The cell cycle state is also a parameter to achieve optimal protein expression. By performing transfections when cells are in G1 phase, plasmids may reach the nuclei in a latter phase of cell cycle (e.g. S or G2/M) that is more favorable to homologous recombination, further contributing to the formation and chromosomal integration of larger plasmid concatemers.

### Protein Secretion

### Characterization of Recombinant IgG Produced by Low and High Producer CHO Clones

First, bottlenecks or defects that may affect the expression and secretion of IgG heavy and light chain by CHO cell clones that display high or low Mab production levels we studied.

Various clones of CHO-K1 S expressing different recombinant IgGs were generated.

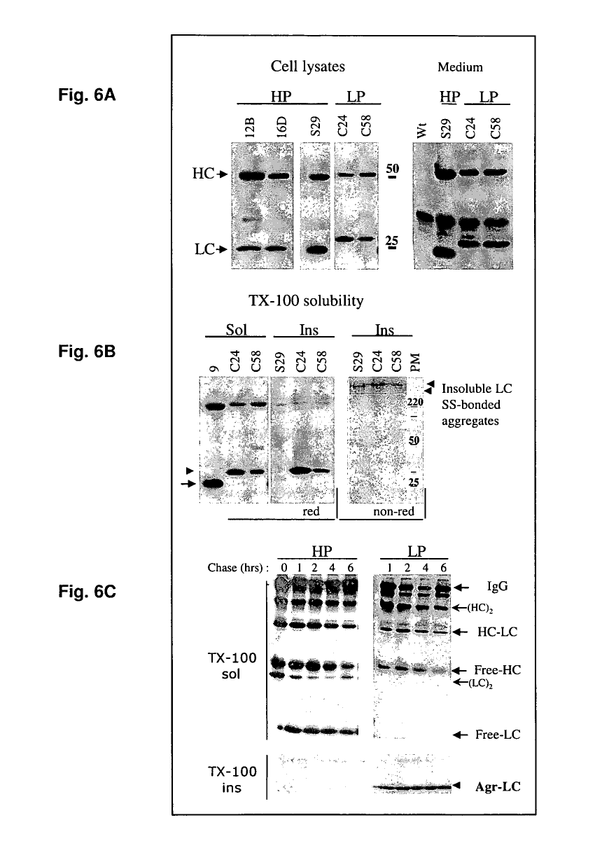

FIG. 6 depicts the characterization of the heavy and light chain of immunoglubilin expressed by high and low recombinant IgG-producers CHO clones. FIG. 6(A) shows a Western blot of intracellular (cell lysates) and secreted IgG (medium) using an anti-human IgG antibody. High (HP) and low (LP) IgG-producers CHO-K1-S were subjected to total cell extraction and analyzed on Laemmli SDS-PAGE 8%. Immunoglobulin heavy and light chain are labeled in the Figure as HC and LC, respectively. FIG. 6(B) depicts a TX-100 solubility analysis. Cells were lysed in PBS containing 1% Triton X100. After centrifugation at 10.000×g for 10 minutes, Tx-soluble proteins containing supernatant and Tx-insoluble proteins containing pellet were resolved under reducing and non-reducing SDS-PAGE 8%. FIG. 6(C) depicts a Cycloheximide-based chase analysis of folding and secretion kinetics of IgG. High (HP) and low (LP) IgG-producers CHO-K1 S clones were cultivated in the presence of 100 μM cycloheximide. At various time points, cells were harvested and lysed in 1% TX-100 containing buffer. Tx-soluble and insoluble fractions were then resolved on non-reducing SDS-PAGE 4-10%. Free, dimer and assembly intermediates complexes of immunoglobulin were labeled as free-HC or free-LC; (LC)₂ and (HC)₂; HC-LC and IgG, respectively. Arrows indicate properly processed structures while arrowhead indicate anomalous structures.

As can be seen from FIG. 6, the Mab titer in the supernatant of cultures culture were highly variable depending on the Mab protein that was overexpressed. However, the results were highly reproducible with some Mabs consistently yielded lowly-producing cell clones while other consistently yielded high producing cell clones. However, the level of expression was unrelated to the plasmid construction used for transfection, and it did not depend on the signal sequence that was used, which was indeed the same for all Mabs (data not shown).

The intracellular heavy and light chains (HC and LC) expressed by each clone were analyzed in order to find a correlation between polypeptide biosynthesis and IgG secretion level of the different clones. Total cell extracts and secreted IgG immuno-precipitates produced by CHO-K1 S clones at steady state were resolved under reducing condition by SDS-PAGE. The protein migration profiles revealed the expected 50 kDa and 25 kDa bands of the HC and LC of high IgG-producer clones 12B, 16D and S29, respectively. However, the light chain expressed by the low IgG-producers C24 and C58 migrated at an abnormally high apparent molecular weight (FIG. 6A). The same anomalous behavior was noted when analyzing the secreted proteins. PNGase F mediated deglycosylation experiments performed on cell extracts and secreted IgG did not alter the LC mobility, indicating that the slow electrophoretic migration of the LC of low producers was not due to the addition glycan moiety (data not shown).

To assess the biochemical nature of the anomalous LC, we extracted the intracellular HC and LC content in PBS solution containing 1% triton X-100 (Tx) and separated the Tx-soluble from the Tx-insoluble proteins by centrifugation at 10.000×g for 10 minutes. After complete protein solubilization in SDS-containing Laemmli's buffer supplemented with urea 9 M, the fractions were resolved by reducing SDS-PAGE. The LC and HC of the high IgG-producer clones were detected only into the Tx-soluble fraction as expected (FIG. 6(B); S29 lanes). However, a significant portion of the LC of the C24 and C58 low producer could not be solubilized by the Tx treatment, indicating intracellular precipitation and potential polypeptide cross-linking. Surprisingly, this Tx-insoluble fraction of the LC could not be resolved under non-reducing condition, as it remained in the stacking gel, indicating high molecular mass aggregates and formation of disulfide bonds. These data suggested a default in the LC folding or assembly process leading to its aggregation in the Tx-resistant form.

Cycloheximide-based chase assays were then performed to investigate the IgG folding and assembly kinetic as well as the fate of the IgG aggregates in the CHO cell clones. SDS-PAGE analysis of the high-producer clone under non reducing condition revealed an accumulation of free LC species and the formation of HC and LC dimers. The HC-containing species appeared to decrease progressively with a concomitant incorporation into HC-LC complexes and in completed IgG tetramers (FIG. 6C). In contrast, LC was detected only within aggregated forms in the low IgG-producer (FIG. 1C, Tx-100 insoluble panel) or incorporated into intermediate complexes of the assembly such as HC-LC and in the completed IgG (FIG. 6C: Tx-100 soluble panel). Interestingly, the amount of detergent-insoluble LC form remained constant over time and thus did not participate in the IgG folding process (FIG. 6C: Tx-100 insoluble panel, Agr-LC). This demonstrated that the LC-aggregates were incompetent for any further folding and assembly process.

These results prompted the following hypotheses: (1) the ER folding machinery and secretion capacity of the high IgG-producers are close to saturation by the large biosynthesis and accumulation of H— and LC, but that the cells could nevertheless proceed with the assembly of normally structured Mab; (2) the accumulation of assembly-incompetent LC aggregates produced by the low IgG-producers explained a secretion defect of these clones; and (3) the potential lack of LC signal peptide cleavage and concomitant aggregation of the LC in low-IgG producers, which might indicate a default of the co-translational translocation of the polypeptide in the ER.

To assess a potential malfunctioning of the ER in the low producer clones, the expression of different sensors of the ER protein folding and stress responses were investigated. Over-expression of recombinant proteins beyond the folding capacity of the ER has been shown to trigger a set of cellular responses collectively called the Unfolded-Proteins-Response (UPR). Although these cellular mechanisms may act to improve the ER folding capacity, to reduce ER stress and to restore ER functionality, they can also reduce the yield of recombinant proteins (Khan S U et al, 2008; Kang S—W et al, 2006). For instance, the activation of ERAD (ER-associated degradation) gene expression upon UPR can target unfolded or misassembled ER-retained recombinant proteins to degradation pathways. Moreover, in the case of severe ER stress, cells that are not able to adapt their protein secretion machineries may trigger the apoptosis pathway.

To assess if LC misprocessing and/or the over-expression of recombinant immunoglobulin chains may induce ER stress and/or UPR, low and high producer clones were cultivated for 7 days and analyzed at various times along the culture procedure.

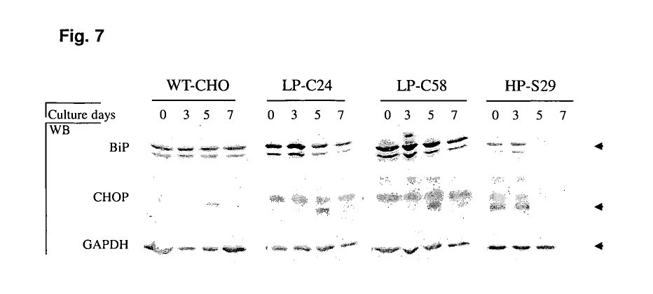

FIG. 7 shows the characterization of the ER folding and UPR machineries of High and Low IgG-producers. High (HP), low (LP) IgG-producers clones and parental cell were cultivated in batch-culture. At various time points, day 0, 3, 5 and 7 of cultivation, cells were harvested. Cell extracts were then analyzed by western blotting using anti-BiP antibody (upper panel) and anti-CHOP antibody (middle panel). Protein loading control was estimated by GAPDH content (bottom panel). CHOP precursor and active forms were indicated by asterisk and arrow respectively.

The Western blot demonstrated an increased expression of BiP, a sentinel marker of UPR activation, in the two low producer clones. In contrast, no increase of BiP level was detected for the high producer clone (FIG. 7). These results suggested that low producers clones expressing a misprocessing LC triggered a ER-stress response mediated by BiP over-expression. In contrast, the low level of BiP protein expressed by high producer clones suggested that these cells can handle and secrete the very high levels of recombinant IgG without activating the UPR cascade.

The expression level of the CHOP pro-apoptotic transcription factor, whose expression can be induced when the protein-folding bottleneck or misfolding cannot be resolved by UPR, was also analyzed. Both the low and high IgG-producer CHO clones exhibited over-expression of CHOP protein when compared to control cells that do not express the Mab (FIG. 7). Interestingly, the CHOP protein progressively accumulated in the two low producers clones up to day 5 of the culture, while the cellular CHOP level and pro-apoptotic pathway seemed to be constitutively elicited in the high producer clone.

These observations implied that a BiP-mediated UPR responses of the low producer clone resulted in the terminal phase of UPR and in the activation of apoptotic cell death programs. In contrast, the high producer clones did not trigger BiP-mediated UPR response, although a CHOP-mediated pro-apoptotic response was induced in these clones. This suggested that the abnormal and huge over-expression of the recombinant Mab may have initiated the cell death programs independently of the main ER stress sensor BiP.

It could also been shown that the different IgG-producer clones exhibited various folding and processing status of the recombinant IgG proteins and that distinct cellular and molecular responses of the host cell were induced during their expression and secretion. Therefore, these various low and high producer clones may both face limitations that may negatively affect industrial production of easy- or difficult-to-express recombinant proteins. We thus went on to use these high and low IgG-producers CHO clones as cellular models to identify novels means to improve recombinant IgG production using bioengineering approaches.

### Strategies for Correcting the Processing of Proteins and Rescue Efficient Secretion

The lack of solubility of the LC in the low producer clones and its slow mobility suggested the presence of peptide signal, and it argued in favor of an inefficient targeting and/or misprocessing of LC pre-proteins to the ER compartment. Thus, it was possible that signal peptide misprocessing and aggregation of the IgG light chain of the recombinant IgG may results from improper targeting and/or translocation into the ER. Recent studies suggested that the bioengineering of the host cell lines to express ER stress related proteins such as BiP could improve secretion of heterologous protein (Peng and M. Fussenegger, 2009). However, this was not an option likely to succeed in our case as ER stress protein BiP was found to be already spontaneously upregulated in the low-producer clones.

Attempts to improve protein secretion by the over-expression of components of the protein translocation machinery have not been met with success in mammalian cell lines. For instance, SR14 expression beyond normal levels did not improve secretion efficiency from cultured human cells (Lakkaraju et al., 2008).

Irrespective of these results, attempts were made to enhance protein secretion by expressing proteins of —or related to— the Signal Recognition Particle (SRP), which is a multiprotein-RNA complex that binds affinity-signal peptide and mediates the docking of SRP-RNA-Ribosome complex onto the ER membrane. Specifically, (1) the human SRP14 subunit, (2) a dominant-negative mutant of the FADD (FAS-associated death domain) protein involved in the regulation of cell apoptosis were expressed, as well as (3) the unrelated GFP protein as a control.

Two clonal cell lines were used, one expressing a low yield monoclonal antibody (e.g. infliximab, a difficult-to-express protein) and one expressing a high yield MAb (e.g. trastuzumab, an easy-to-express protein) harbouring the same signal peptide, and 5.1×10⁵ cells were re-transfected with 5 μg of plasmid encoding the indicated proteins by electroporation (MICROPORATOR, 1250V, 20 ms pulse time and 3 pulses). After microporation, the cells were added to SFM4CHO medium (HYCLONE) supplemented with 8 mM glutamine and 2×HT. Two days later, the cells were transferred in T75 plates at an appropriate dilution of the selection marker (300 μg/ml G418) and the cells were further cultured. After approximately two weeks, drug-resistant cells were expanded in shake flask and the SRP14-expressing populations were diluted for single-cell cloning in a limiting dilution process. Results presented below were generated with cell clones expressing the indicated proteins.

### Enhancement of Mab Production by SRP14 Expression

Firs, the Tx solubility of intracellular HC and LC was determined and the secretion level of the high and low IgG-producers clones expressing the SRP-related proteins.

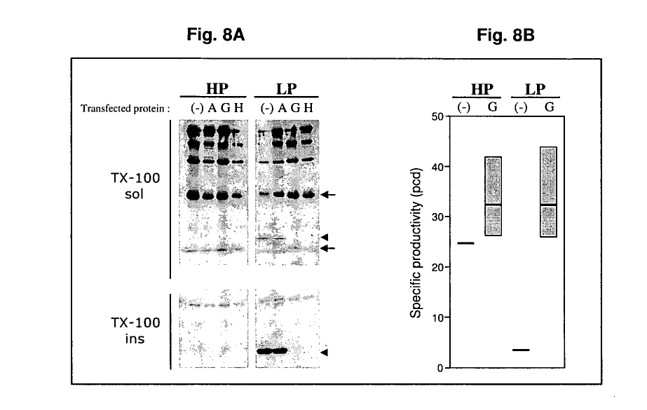

FIG. 8 shows that SRP14 transfection of recombinant IgG producing CHO clones abolished light chain aggregation and rescued IgG secretion. Two differents CHO clones, the high (F9) and low (A37) recombinant IgG producing CHO clones, were subjected to transfection with cDNA constructions coding for various control or candidate proteins expected to rescue the correct processing and secretion of recombinant IgG (A: GFP; G: SRP14; H: FADD-DN).

In FIG. 8(A) TX-100-soluble and -insoluble fraction of cell extracts of low and High producers CHO clones co-expressing proteins A, G or H were analyzed on 4%-10% gradients SDS-PAGE and IgG proteins detected by chemiluminescence. Light arrows show the free and unprocessed-LC produced by low IgG producers (light arrow head) and the unprocessed-aggregated LC. The free and properly processed LC produced by low IgG producer clone after transfection of G or H proteins were labeled by black arrow heads.

FIG. 8(B) shows the specific productivity distribution of F9 and A37 clones before (−) and after (G) transfection with the SRP14 expression vector, as assessed from ELISA assays of secreted Mabs performed on cell culture medium supernatants.

Interestingly, the western blot analysis indicated that the over-expression of full length human SRP14 (Genebank access number X73459.1, which is incorporated herein by reference in its entirety) led to the conversion of the pro-LC into a species migrating like the normal LC mature form competent for folding and assembly with the HC, while the migration of HC was not affected (FIG. 8A, lane G of top panel, see arrows). Even more strikingly, SPR14 over-expression fully abolished LC aggregation in the TX-insoluble fraction (FIG. 8A, bottom panel). Expression of the control GFP protein did not improve protein solubility, nor did it restore proper processing of the LC (FIG. 8A, lane A). Expression of SRP14 had no effect on the HC and LC migration pattern obtained from the high producer clone, but the amount of the free HC and LC and of fully assembled IgG was somewhat increased as compared to the controls (FIG. 8A, FP-F9 clone, lane G).

To determine the consequence of exogenous expression of SRP14 on recombinant IgG titer, supernatant cell culture were then analyzed by ELISA to probe for properly assembled Mab. The over-expression of SRP14 in low IgG-producers lead to a significant increase in IgG secretion from the low producer clone (FIG. 3B). Clones isolated from the low producer cells (LP-G population) exhibited an average increase in specific productivity (Qp) of 7-fold. Moreover, the exogenous expression of SRP14 did also improve the secretion of the HP clone as a 30% increase in Qp could be observed (HP-G clones). Interestingly, individual clones were isolated that could express the IgG at identical level for the difficult and easy to express Mab (>40 pcd), suggesting that subsequent steps in translocation become rate limiting

The action of the exogenous SRP14 expression is unexpected. The expression may have caused an extended delay of the LC elongation in the difficult-to-produce IgG producer clones, given the function of this subunit in the elongation arrest mediated by SRP. Proper processing the Mabs of the low producer clones may require an unexpectedly long translational pausing, possibly because the kinetics of docking of the complex mediating the translocation of these particular IgGs onto the ER may be slower than that of other secreted proteins. Modulation of the translation kinetic by the exogenous SRP14 components could in return influence the co-translocation of the pro-LC in the ER and thus restore the efficient processing of the signal peptide.

The effect of another control protein, namely the FAS-associated protein with death domain (FADD), was also evaluated by expressing a dominant negative mutant of FADD (FADD-DN) (Newton, Harris et al. 1998). Unexpectedly, the over-expression of FADD-DN was also found to abolish LC aggregation, as found for SRP14 (FIG. 8A, bottom panel, lane H). This was not expected because FADD is known to associate to the family of Death Receptor (DR) proteins that induce apoptosis in cells by forming the death-inducing signaling complex (DISC). A main physiological role of cytoplasmically located FADD is thus to trigger cell death, and attempt were therefore made to inactivate FADD to prevent CHO cell apoptosis. However, recent work has ascribed multiple non-apoptotic activities to FADD, depending on modifications and subcellular localization. For instance FADD phosphorylation and nuclear entry regulate gene expression and activate both the cell cycle and mitotic progression (Tourneur and Chiocchia 2010). Furthermore, the ER-bound protein termed RTN3 can recruit FADD to the ER membrane, and FADD itself can be secreted by an atypical microvesicle-based pathway. However, so far, FADD had not been implicated in the regulation of protein secretion via the ER. Our finding might therefore indicate a novel function for FADD in the context of improving the processing of over-expressed Mabs. Alternatively, expression of FADD-DN may have saturated the translation machinery, somehow slowing down this process and allowing proper targeting to the ER. However, neither FADD-DN nor GFP over-expression was found to significantly restore IgG secretion at a high level. Thus, only the exogenous expression of SRP14 was capable of restoring Mab production. Thus, it was hypothesized that the modulation of SRP-complex functions might by specifically needed for the recruitment of ER-lumen translocon partners and/or for the interaction of the neosynthetized LC with ER folding chaperones. Very high Mab secretion levels were maintained for more than 6 months, indicating that it is a stable property of SRP14-expressing cells. In fed-batch cultures, more than 8 grams of Mab per liter of culture medium were obtained, which is a great titer that we had not achieved without SRP14 expression for this difficult-to-express protein.

The good results obtained after the expression of SRP14 prompted the testing of the effect of other proteins that may contribute to proper translocation of nascent polypeptides in the ER. Other proteins of the Signal Recognition Particle (SRP), which is a multiprotein-RNA complex that binds affinity-signal peptide and mediates the docking of SRP-RNA-Ribosome complex onto the ER membrane, or proteins that relate to SRP function were also tested. Specifically, we expressed (1) the human SRP54 subunit, in an attempt to augment the signal sequence recognition, (2) the human SRP9 and/or SRP14 subunit, as these two polypeptides form a complex in vivo, to possibly slow down translation, (3) the human SRP receptor (SR) subunits α and β attempting to increase the capacity of the translocation machinery, and (4) the translocons Sec61 human subunits (Transl), to possibly improve translocation in the ER.

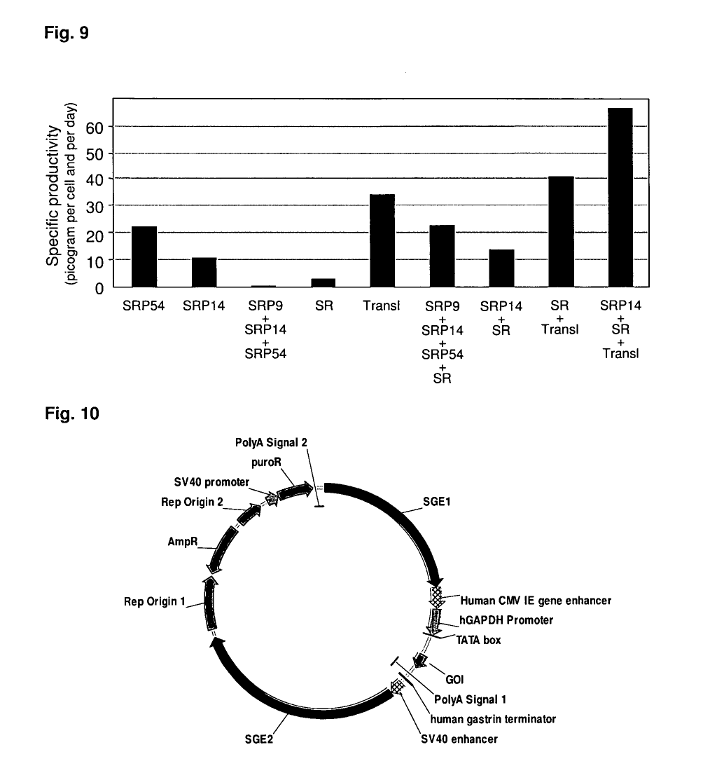

FIG. 9 depicts the increase in MAb production in CHO cell pools expressing various combinations of SRP9, SRP14, SRP54, SR and Translocon. The low producer A37 clone was subjected to transfection with cDNA constructions driving the expression of the indicated candidate proteins. Culture supernatant were analyzed by ELISA, and the titers of Mab secretion was determined

As can be seen from the figure, expression of SRP14 or SRP54 led to a strong increase in Mab secretion, whereas SR and Transl led to a smaller but still significant increase of secretion, while SRP9 alone did not significantly improve expression (FIG. 9 and data not shown). Combinations of these proteins were also tested. SRP9 fully abolished the positive effects obtained by the expression of SRP14 and/or 54 (SRP9+SRP14+SRP54 lane), indicating it is not the simple expression of any SRP protein that leads to improved secretion. SR expression modestly increased the effects mediated by SRP14 and Transl alone, however it strongly increased secretion obtained in presence of SRP 9, 14 and 54 (compare SRP9+SRP14+SRP54 lane with SRP9+SRP14+SRP54+SR lane). However, the highest gain in secretion was obtained when over-expressing Transl in addition to SRP14 and SR(SRP14+SR+Transl vs SRP14+SR). It will be obvious to a skilled-in-the-art individual that other combinations of SRP14, SRP54, SR and Transl will also contribute to improve protein secretion, and that all such combinations are therefore embodied in the present invention.

### Material and Methods

### 1. Transgene Integration and Expression

### Plasmids and Constructs

pGEGFPcontrol contains the SV40 early promoter, enhancer and vector backbone from pGL3 (PROMEGA) driving the expression of eGFP gene from pEGFP-N1 (CLONTECH). pPAG01SV40EGFP results from the insertion of the chicken lysozyme MAR fragment upstream of the SV40 early promoter of pGEGFPcontrol (Girod et al. 2005). The human MAR 1-68 was identified by the SMARScan program using DNA structural properties. It was cloned from human bacterial artificial chromosomes in pBluescript and then inserted into pGEGFPcontrol upstream the SV40 early promoter, resulting in the p1-68 (NcoI filled) SV40EGFP plasmid (Girod et al. 2007). pGL3-CMV-DsRed was created by inserting the DsRed gene, under the control of the CMV promoter (including the enhancer), from pCMV-DsRed (CLONTECH) in pGL3-basic (PROMEGA). pGL3-CMV-DsRed-kan was then created by exchanging the ampicillin gene of pGL3-CMV-DsRed for kanamycin resistance gene from pCMV-DsRed (CLONTECH) by digestion of both plasmids with BspHI. Then, the chicken lysozyme or the human 1-68 MAR were inserted into the pGL3-CMV-DsRed-kan plasmid. They were inserted as KpnI/BgIII fragment containing the chicken lysozyme fragment, or as KpnI/BamHI human 1-68MAR fragment, upstream of the CMV promoter in pGL3-CMV-DsRed-kan, resulting in pPAG01GL3-CMV-DsRed and p1-68(NcoI) filled GL3-CMV-DsRed, respectively.

### Cell Culture and Transfection

The CHO DG44 cell line (Urlaub 1983) was cultivated in DMEM: F12 (GIBCO) supplemented with HT (GIBCO) and 10% foetal bovine serum (FBS, GIBCO). Parental CHO cells AA8, NHEJ deficient cells V3.3 and HR deficient cells 51D1 (Adayapalam et al., 2008) were kindly provided by Dr. Fabrizio Palitti and were cultivated in DMEM: F12 medium with 10% foetal bovine serum and antibiotics.

Transfections were performed with these cells using Lipofect-AMINE 2000, according to the manufacturer's instructions (INVITROGEN). GFP or DsRed fluorescence levels were analyzed using a fluorescence-activated cell sorter (FACS), one, two or three days post transfection (transient transfections). Stable pools of CHO-DG44 cells expressing GFP and/or DsRed were obtained by cotransfection of the resistance plasmid pSVpuro (CLONTECH). After two weeks of selection with 5 μg/ml puromycin for CHO-DG44 (8 μg/ml puromycin for AA8, V3.3 and 51D1), cells were analyzed by FACS. Multiple transfection were performed as follows: after first transfection, the cells were then transfected a second time as described above, except that the resistance plasmid carried another resistance gene, pSV2 neo (CLONTECH). The two transfections were timed to follow the cell cycle, unless otherwise indicated in the text. Twenty-four hours after the second transfection, cells were passaged and selected with 250 μg/ml G418 and/or 2.5 μg/ml puromycin (250 μg/ml G418 and 4 μg/ml puromycin for AA8, V3.3 and 5A1 D1). After three weeks of selection, GFP and/or DsRed expression was analyzed by FACS.

### Fluorescence Activated Cell Sorting

Transient expression of eGFP and DsRed proteins was quantified at 24 h, 48 h or 72 h after transfection using a FACScalibur flow cytometer (BECTON DICKINSON), whereas expression of stable cell pools was determined after at least 2 weeks of antibiotic selection. Cells were washed with PBS, harvested in trypsin-EDTA, pooled, and resuspended in serum-free synthetic ProCHO5 medium (CAMBREX corporation). Fluorescence analyses were acquired on the FACScalibur flow cytometer (BECTON DICKINSON) with the settings of 350V on the GFP channel (FL-1) and 450V on the DsRed channel (FL-3) for transient expression, whereas settings of 240V for FL-1 and 380V for FL-3 were used to analyze stable expression. 100′000 events were acquired for stable transfections and 10′000 for transient transfections. Data processing was performed using the WinMD software.

### Cell Cycle Analysis

At the indicated times, the cell cycle status was analyzed by flow cytometry of CHO cells after staining of the DNA with propidium iodide (PI). Cells were first washed with a (PBS), trypsinized and harvested in 1 ml of growth media by centrifugation for 5 min at 1500 rpm in a microcentrifuge. After an additional PBS wash, cells were resuspended in 1 ml of PBS before fixing with ethanol by the addition of 500 μl of cold 70% ethanol dropwise to the cell suspension under agitation in a vortex. Samples were incubated for 30 minutes at −20° C. and cells were centrifuged as before. The resulting cell pellet was resuspended in 500 ml of cold PBS, supplemented with 50 μg/ml of RNaseA and DNA was stained with 40 μg/ml of PI for 30 minutes in the dark. Cells were then washed with PBS, centrifuged and resuspended in 500 μl of ProCHO5 medium (CAMBREX corporation) before analysis in a FACScalibur flow cytometer (FL-3 channel; BECTON DICKINSON). 10′000 events were acquired for each sample.

### Fluorescent in Situ Hybridization

FISH (Fluorescent In Situ Hybridization) were performed as described in Derouazi et al. (2006) and Girod et al. (2007). Briefly, metaphase chromosomal spreads were obtained from cells transfected with or without the 1-68 human MAR and treated with colchicine. Fluorescence in situ hybridization was performed using hybridization probes prepared by the direct nick translation of pSV40GEGFP plasmid without the MAR.

### Isolation of Nuclei and DNA

Nuclei were isolated one, two or three days after transient transfection(s), from proliferating and confluent CHO DG44 cells grown in 6-well plates. 1×10⁶ cells were washed twice with cold PBS, resuspended in 2 volumes of cold buffer A (10 mM HEPES (pH 7.5), 10 mM KCl, 1.5 mM Mg(OAc)₂, 2 mM dithiothreitol) and allowed to swell on ice for 10 min. Cells were disrupted using a Dounce Homogeniser. The homogenate was centrifuged for 2 min at 2000 rpm at 4° C. The pellet of disrupted cells was then resuspended in 150 μl of PBS and deposited on a cushion of Buffer B (30% sucrose, 50 mM Tris-HCl (pH 8.3), 5 mM MgCl₂, 0.1 mM EDTA) and centrifuged for 9 min at 1200 g. The pellets of nuclei were resuspended in 200 μl of Buffer C (40% glycerol, 50 mM Tris-HCl (pH 8.3), 5 mM MgCl₂, 0.1 mM EDTA) and stored frozen at −80° C. until required (Milligan et al. 2000).

Total cell DNA was isolated from CHO DG44 stable cell pools or from isolated cell nuclei using the DNeasy Tissue Kit from QIAGEN. For stable cell pools, 1×10⁶ confluent CHO DG44 cells growing in 6-well plates were collected. DNA extraction was performed according to the manufacturer's instruction for the isolation of total DNA from cultured Animal cells. For isolated cell nuclei, frozen pellets of nuclei were first thawed and centrifuged at 300 g for 5 min to remove Buffer C before beginning DNA extraction following the same protocol as for stable cell lines.

### Transgene Copy Number Determination and Quantitative PCR

To determine the copy number of transgenes integrated in the genome, approximately 6 ng of genomic DNA were analyzed by quantitative PCR using the SYBR Green-Taq polymerase kit from EUROGENTEC Inc. and ABI Prism 7700 PCR machine. The following primers were used to quantify GFP DNA: GFP-For: ACATTATGCCGGACAAAGCC and GFP-Rev: TTGTTTGGTAATGATCAGCAAGTTG, while primers GAPDH-For: CGACCCCTTCAT-TGACCTC and GAPDH-Rev: CTCCACGACATACTCAGCACC were used to amplify the GAPDH gene. The ratios of the GFP target gene copy number were calculated relative to that of the GAPDH reference gene as described previously (Karlen et al. 2007). To determine transgene import into nuclei after transfection, quantitative PCR was performed on DNA extracted from purified nuclei using the same GFP and GAPDH primer pairs as above.

The number of GAPDH gene and pseudogene copies used as reference was estimated for the mouse genome, as the CHO genome sequence is not available as yet. Alignment were performed by BLAST analysis performed using the NCBI software of the DNA sequence of the 190 bp amplicon generated by the GAPDH primers on the mouse genome, which gave a number of 88 hits per haploid genome. As the CHO DG44 are near-diploid cells (Derouazi et al. 2006), we estimate that 176 copies of the GAPDH genes and pseudogenes occur in the genome of CHO DG44 cells. This number was used as a normalization reference to determine the GFP transgene copy number.

### Confocal Microscopy

pGEGFPcontrol and p1-68 (NcoI filled) SV40EGFP plasmids were labelled either with rhodamine by the Label IT Tracker TH-Rhodamine Kit or with Cy5 by the Label IT Tracker Cy 5 Kit (MIRUS, MIRUSBIO) according to the manufacturer's protocol, and purified by ethanol precipitation. For transfection, DNA transfection was carried out with the Lipofectamine 2000 Reagent (INVITROGEN) according to the supplier's instructions. At 3, 6 and 21 h after transfection, the medium was removed and the cells were fixed with 4% paraformaldehyde at room temperature for 15 min. When indicated, cells were treated for 30 min with LysoTracker™ Red DND-99 (Molecular Probes, INVITROGEN) at a final concentration of 75 nM before the fixation, to stain the acidic organelles (e.g., endosomes and lysosomes) according to the manufacturer's instructions. The fixed cells were then washed twice with PBS and mounted in a DAPI/Vectashied solution to stain the nuclei.

Fluorescence and bright-field images were captured using a CARL ZEISS LSM 510 Meta inverted confocal laser-scanning microscope, equipped with a 63×NA 1.4 planachromat objective. Z-series images were obtained from the bottom of the coverslip to the top of the cells. Each 8-bit TIFF image was transferred to the ImageJ software to quantify the total brightness and pixel area of each region of interest. For data analysis, the pixel areas of each cluster in the cytosol sᵢ(cyt), nucleus sᵢ(nuc) and lysosome sᵢ(lys) were separately summed in each XY plane. Theses values (S′_{Z=j}(cyt), S′_{Z=j}(nuc) and S′_{Z=j}(lys), respectively) were further summed through all of Z series of images and denoted S(cyt), S(nuc) and S(lys), respectively. The total pixel area for the clusters of labelled pDNA in the cells, S(tot), was calculated as the sum of S(cyt), S(nuc) and S(lys). The fraction of pDNA in each compartment was calculated as F(k)=S(k)/S(tot), where represents each subcellular compartment (nucleus, cytosol or lysosome).

### II. Transgene Expression Product Secretion

### Plasmids and Constructs

The expression vectors contain the bacterial beta-lactamase gene from Transposon Tn3 (AmpR), conferring ampicillin resistance, and the bacterial CoIE1 origin of replication. As derivatives of pGL3 Control (PROMEGA), the terminator region of the vector bears a SV40 enhancer positioned downstream the SV40 polyadenylation signal. A human gastrin terminator has been inserted between the SV40 polyA signal and the SV40 enhancer. Each vector also includes two human 1_{—}68 SGE flanking the expression cassette and an integrated puromycin resistance gene under the control of the SV40 promoter. All the vectors encode the GOI under the control of the hGAPDH promoter (FIG. 10).

The different cloned transgenes were amplified by PCR using the Pwo SuperYield DNA Polymerase Kit following the manufacturer's instructions (ROCHE), human universal cDNA as template (BioChain®) and specific primers (MICROSYNTH AG, Switzerland, see Table 1) for the 5′ and 3′ ends of the CDS with 5′ tails carrying a compatible restriction site for the cloning into the expression vector.

The PCR product and the expression vector were digested by the appropriate restriction enzymes (NEW ENGLAND BIOLABS or PROMEGA). The digested DNA were electrophoresed on a 1% w/v agarose (EUROBIO, CHEMIE BRUNSCHWEIG AG) gel. The vector band and the digested PCR product were cut out of the gel by visualization under preparative UV lamp that does not damage the DNA (UL-6L, VILBER LOURMAT), transferred into a 1.5 mL microtube and purified using standard techniques (WIZARD SV Gel and PCR CleanUp System™, PROMEGA) following the manufacturer's instructions.

Both purified fragments (the digested Selexis™ expression vector and PCR product) were ligated together using LigaFast™ Rapid DNA Ligation System (PROMEGA) in a final volume of 10 μL for 5 min at RT (=Room Temperature) following the manufacturer's instructions. The whole ligation mixture was used to transform 50 μL of competent DH5 alpha cells (INVITROGEN) following the manufacturer's instructions. The integrity and proper structure of the newly created plasmid was checked by restriction analysis. One bacterial clone was expanded in 5 mL of LB+100 μg/mL ampicillin in shake tube for bulk extraction of plasmid DNA. The plasmid was extracted using WIZARD Plus SV Minipreps kit (PROMEGA) following the manufacturer's instructions. The integrity of the plasmid was confirmed by sequencing the GOI and associated flanking sequences.

Upon confirmation, a maxipreparation of the vector was done with a standard DNA isolation kit (JETSTAR 2.0, GENOMED) from a 150 mL overnight culture in LB supplemented with 100 μg/mL ampicillin to obtain very pure plasmid DNA. After purification the DNA was resuspended in 300 μL sterile deionized water. Linearization was performed by Pvul digestion and DNA quantification was conducted using Quant-iT PicoGreen dsDNA assay kit (INVITROGEN/Molecular Probes).

TABLE 1
Primers used in the PCR reactions to amplify the different transgenes of interest

| Primer Name | Primer Sequence (5′ to 3′) | Comment |
|---|---|---|
| hSRP9_Fw_HindIIIfilled | TACCGCCACCATGCCGCAGTACCAGACCTGGGA | PCR of hSRP9 |
| hSRP9_Rv_XbaI | CTAGTCTAGATCAGGAGGCTGAAGCAAGAGGA | (NM_001130440.1) |
| hSRP14_Rv_NcoI | TCATGCCATGGTGTTGTTGGAGAGCGAGCA | PCR of SRP14 |
| hSRP14_Rv_XbaI | CTAGTCTAGATTACTGTGCTGCTGTTGCTGCT | (human variant of |
| | | NM_001131937) |
| hSRP54_Fw_NcoI | TCATGCCATGGTTCTAGCAGACCTTGGAAGA | PCR of hSRP54 |
| hSRP54_Rv_XbaI | CTAGTCTAGATTACATATTATTGAATCCCATCA | (NM_003136.3) |
| hSRPRalpha_Fw_HindIII | TCCCAAGCTTACCGCCACCATGCTCGACTTCTTCACCATTTTCT | PCR of |
| hSRPRbeta_Rv_XbaI | CTAGTCTAGATCAGGCAATTTTAGCCAGCCA | hSRPRalpha |
| | | (NM_003139.3) |
| hSRPRbeta_Fw_NcoI | TCATGCCATGGCTTCCGCGGACTCGCG | PCR of |
| hSRPRbeta_Rv_XbaI | CTAGTCTAGATCAGGCAATTTTAGCCAGCCA | hSRPRbeta |
| | | (NM_021203.3) |
| hSEC61A1_Fw_HindIII | TCCCAAGCTTACCGCCACCATGGCAATCAAATTTCTGGAAGTCA | PCR of |
| hSEC61A1_Rv_XbaI | CTAGTCTAGATCAGAAGAGCAGGGCCCCCATGCT | hSEC61A1 |
| | | (NM_013336.3) |
| hSEC61B_Fw_HindIII | TCCCAAGCTTACCGCCACCATGCCTGGTCCGACCCCCAGT | PCR of hSEC61B |
| hSEC61B_Rv_XbaI | CTAGTCTAGACTACGAACGAGTGTACTTGCCCCAA | (NM_006808.2) |
| hSEC61G_Fw_HindIII | TCCCAAGCTTACCGCCACCATGGATCAGGTAATGCAGTTTGTTGA | PCR of hSEC61G |
| hSEC61G_Rv_XbaI | CTAGTCTAGATCAGCCACCAACAATGATGTTATT | (NM_014302.3) |

### Transfection of CHO Cells and Selection of the Stable Transfectants

CHO cells were passaged one day prior to transfection at a density of 300,000 cell/ml. On the day of transfection, the cells were counted and 510,000 cells were harvested by centrifugation. The supernatant was removed and the cell pellet was resuspended in 30 ul of resuspension buffer (Buffer R, INVITROGEN). Four micrograms of linearized plasmid encoding one protein to be tested was added to the cells and the cells were electroporated using the Microporator-mini device from DIGITAL BIO TECHNOLOGY. The settings used for electroporation were 1230 volts, 20 us and 3 pulses.

The electroporated cells were cultured in 6 well plate containing 3 ml of culture medium (SFM4CHO, Hyclone™) supplemented with 8 mM glutamine and 2×HT. One day post-transfection, the selection of stable transfectants was started by adding 500 μg/ml of G418 to the medium. At day three of culture, the cells were harvested by centrifugation and the medium was renewed with 10 ml of fresh culture medium supplemented with antibiotics. After a week, 1.5×10⁶ cells were transferred into a 50 ml minireactor tube (TBS) containing 5 ml of culture medium supplemented with antibiotics and incubated in a shaking incubator. The culture was maintained by passaging twice a weak. At the time of sub-cultivation, the number of cells was recorded and the concentration of the product was determined by ELISA. Those numbers were used to calculate the specific productivity in order to compare the effect of the different protein tested.

Although the invention is illustrated and described in detail on the basis of the Figures and the corresponding description, this illustration and this detailed description are to be understood to be illustrative and exemplary and not as restricting the invention. It is self-evident that a person skilled in the art can make changes and adaptations without leaving the scope of the following claims. In particular, the invention also comprises embodiments with any combination of features which are mentioned herein in connection with different embodiments.

### BIBLIOGRAPHY

Agarwal et al. (1998). Scaffold attachment region-mediated enhancement of retroviral vector expression in primary T cells. J. Virol.; 72: 3720-3728.
Akita et al. (2007). Cell cycle dependent transcription, a determinant factor of heterogeneity in cationic lipid-mediated transgene expression. J Gene Med, 9, 197-207.
Allen et al. (1996) High-level transgene expression in plant cells: effects of a strong scaffold attachment region from tobacco. Plant Cell 8, 899-913.
Allen et al. (2002). DNA-dependent protein kinase suppresses double-strand break-induced and spontaneous homologous recombination. Proc Natl Acad Sci USA, 99, 3758-3763.
Bell, A. C. and Felsenfeld, G. (1999) Stopped at the border: boundaries and insulators. Curr Opin Genet Dev, 9, 191-198.
Bendix-Waltes R, Kalb R, Stumm M. (2005) Rad50 deficiency causes a variant form of Nijmegen breakage syndrome. Eur J Hum Genet. 13: 63.
Benson et al. (1994). Purification and characterization of the human Rad51 protein, an analogue of E. coli RecA. Embo J 13, 5764-5771.
Bernstein, K. A., and Rothstein, R. (2009) At loose ends: resecting a double-strand break. Cell 137, 807-810.
Boulton S J, Jackson S P (1996). Identification of a Saccharomyces cerevisiae Ku80 homologue: Role in DNA double strand break rejoining and in telemetric maintenance. Nucleic Acids Research 24:4639-4648.
Buck et al. (2006). Cernunnos, a novel nonhomologous end-joining factor, is mutated in human immunodeficiency with microcephaly. Cell 124, 287-299.
Bugreev et al. (2007). Rad54 dissociates homologous recombination intermediates by branch migration. Nat Struct Mol Biol 14, 746-753.
Burma et al. (2001). ATM phosphorylates histone H2AX in response to DNA double-strand breaks. J Biol Chem 276, 42462-42467.
Canman et al. (1998). Activation of the ATM kinase by ionizing radiation and phosphorylation of p53. Science 281, 1677-1679.
Capp et al. (2007). Involvement of DNA polymerase mu in the repair of a specific subset of DNA double-strand breaks in mammalian cells. Nucleic Acids Res 35, 3551-3560.
Castilla et al. (1998). Engineering passive immunity in transgenic mice secreting virus-neutralizing antibodies in milk. Nat Biotechnol 16, 349-354.
Chen et al. (1997). Cell cycle-dependent protein expression of mammalian homologs of yeast DNA double-strand break repair genes Rad51 and Rad52. Mutat Res 384, 205-211.
Chung et al. (1993). A 5′ element of the chicken betaglobin domain serves as an insulator in human erythroid cells and protects against position effect in Drosophila. Cell 74, 505-514.
Clikeman et al. (2001). Homologous recombinational repair of double-strand breaks in yeast is enhanced by MAT heterozygosity through yKU-dependent and -independent mechanisms. Genetics 157, 579-589.
Connolly et al. (1991). Requirement of GTP hydrolysis for dissociation of the signal recognition particle from its receptor. Science 252(5009): 1171-1173.
D'Amours, D., and Jackson, S. P. (2002). The Mre11 complex: at the crossroads of dna repair and checkpoint signalling. Nat Rev Mol Cell Biol 3, 317-327.
Darroudi, F., Wiegant, W., Meijers, M., Friedl, A. A., van der Burg, M., Fomina, J., van Dongen, J. J., van Gent, D. C., and Zdzienicka, M. Z. (2007). Role of Artemis in DSB repair and guarding chromosomal stability following exposure to ionizing radiation at different stages of cell cycle. Mutat Res 615, 111-124.
Davies, O. R., and Pellegrini, L. (2007). Interaction with the BRCA2 C terminus protects RAD51-DNA filaments from disassembly by BRC repeats. Nat Struct Mol Biol 14, 475-483.
De Jager et al. (2001). Human Rad50/Mre11 is a flexible complex that can tether DNA ends. Mol Cell 8, 1129-1135.
DeFazio et al. (2002). Synapsis of DNA ends by DNA-dependent protein kinase. Embo J 21, 3192-3200.
Delacote, F., and Lopez, B. S. (2008). Importance of the cell cycle phase for the choice of the appropriate DSB repair pathway, for genome stability maintenance: the trans-S double-strand break repair model. Cell Cycle 7, 33-38.
Delacote et al. (2002) An xrcc4 defect or Wortmannin stimulates homologous recombination specifically induced by double-strand breaks in mammalian cells. Nucleic Acids Res, 30, 3454-3463.
Derouazi et al. (2006) Genetic characterization of CHO production host DG44 and derivative recombinant cell lines. Biochem Biophys Res Commun, 340, 1069-1077.
Downs, J. A., and Jackson, S. P. (2004). A means to a DNA end: the many roles of Ku. Nat. Rev Mol Cell Biol 5, 367-378.
Dronkert et al. (2000). Mouse RAD54 affects DNA double-strand break repair and sister chromatid exchange. Mol Cell Biol 20, 3147-3156.
Dupre et al. (2006). Two-step activation of ATM by DNA and the Mre11-Rad50-Nbs1 complex. Nat Struct Mol Biol 13, 451-457.
Esashi, et al. (2007). Stabilization of RAD51 nucleoprotein filaments by the C-terminal region of BRCA2. Nat Struct Mol Biol 14, 468-474.
Folger et al. (1985) Nonreciprocal exchanges of information between DNA duplexes coinjected into mammalian cell nuclei. Mol Cell Biol, 5, 59-69.
Fusco, C., Reymond, A., and Zervos, A. S. (1998). Molecular cloning and characterization of a novel retinoblastoma-binding protein. Genomics 51, 351-358.
Galbete et al. (2009) MAR elements regulate the probability of epigenetic switching between active and inactive gene expression. Mol Biosyst, 5, 143-150.
Gilmore, R., G. Blobel, et al. (1982). “Protein translocation across the endoplasmic reticulum. I. Detection in the microsomal membrane of a receptor for the signal recognition particle.” J Cell Biol 95(2 Pt 1): 463-9.
Gilmore, R., P. Walter, et al. (1982). “Protein translocation across the endoplasmic reticulum. II. Isolation and characterization of the signal recognition particle receptor.” J Cell Biol 95(2 Pt 1): 470-7.
Girod et al. (2007) Genome-wide prediction of matrix attachment regions that increase gene expression in mammalian cells. Nat Methods, 4, 747-753.
Girod et al. (2005) Use of the chicken lysozyme 5′ matrix attachment region to generate high producer CHO cell lines. Biotechnol Bioeng, 91, 1-11.
Golzio et al. (2002) Cell synchronization effect on mammalian cell permeabilization and gene delivery by electric field. Biochimica et Biophysica Acta (BBA)-Biomembranes, Volume 1563 (1-2), Pages 23-28.
Gottlieb, T. M., and Jackson, S. P. (1993). The DNA-dependent protein kinase: requirement for DNA ends and association with Ku antigen. Cell 72, 131-142.
Grawunder et al. (1997). Activity of DNA ligase IV stimulated by complex formation with XRCC4 protein in mammalian cells. Nature 388, 492-495.
Greenberg et al. (2006). Multifactorial contributions to an acute DNA damage response by BRCA1/BARD1-containing complexes. Genes Dev 20, 34-46
Grosjean et al. (2002). Correlation between CHO cell cycle and transfection effiency using Ca/PO4. Cytotechnology 38.
Hart, C. M. and Laemmli, U. K. (1998) Facilitation of chromatin dynamics by SARs. Curr Opin Genet Dev, 8, 519-525.
Henikoff, S. (1996) Dosage-dependent modification of position-effect variegation in Drosophila. Bioessays, 18, 401-409.
Hinz et al. (2006) Repression of mutagenesis by Rad51D-mediated homologous recombination. Nucleic Acids Res, 34, 1358-1368.
Ira et al. (2004). DNA end resection, homologous recombination and DNA damage checkpoint activation require CDK1. Nature 431, 1011-1017.
Jackson, D. A. (1997) Chromatin domains and nuclear compartments: establishing sites of gene expression in eukaryotic nuclei. Mol Biol Rep, 24, 209-220.
Jayathilaka et al. (2008) A chemical compound that stimulates the human homologous recombination protein RAD51. PNAS 105 (41), 15848-15853.
Jeggo, P. A. (1997) DNA-PK: at the cross-roads of biochemistry and genetics. Mutat Res, 384, 1-14.
Johansson et al. (1993). Positively charged amino acids placed next to a signal sequence block protein translocation more efficiently in Escherichia coli than in mammalian microsomes. Mol Gen Genet. 239(1-2), 251-6.
Kalos, M. and Fournier, R. E. (1995) Position-independent transgene expression mediated by boundary elements from the apolipoprotein B chromatin domain. Mol Cell Biol, 15, 198-207.
Karlen et al. (2007). Statistical significance of quantitative PCR. BMC Bioinformatics, 8, 131.
Kaufman, R. J. (2000). Overview of vector design for mammalian gene expression. Mol Biotechnol, 16, 151-160.
Keenan et al. (2001). The signal recognition particle. Annu Rev Biochem 70, 755-75.
Kim et al. (2004). Improved recombinant gene expression in CHO cells using matrix attachment regions. J Biotechnol, 107, 95-105.
Kim et al. (2006). Signaling networks controlled by the MRN complex and MDC1 during early DNA damage responses. Mol Carcinog 45, 403-408.
Koch et al. (2004). Xrcc4 physically links DNA end processing by polynucleotide kinase to DNA ligation by DNA ligase IV. Embo J 23, 3874-3885.
Krappmann et al. (2006) Eukaryot. Cell. 5:212-215.
Lakkaraju, A. K., C. Mary, et al. (2008). “SRP keeps polypeptides translocation-competent by slowing translation to match limiting ER-targeting sites.” Cell 133(3): 440-51.
Lavin, M. F. (2007). ATM and the Mre11 complex combine to recognize and signal DNA double-strand breaks. Oncogene 26, 7749-7758.
Lee et al. (2004). Implication of DNA polymerase lambda in alignment-based gap filling for nonhomologous DNA end joining in human nuclear extracts. J Biol Chem 279, 805-811.
Lee et al. (1997). Evidence for DNAPK-dependent and -independent DNA double-strand break repair pathways in mammalian cells as a function of the cell cycle. Mol Cell Biol 17, 1425-1433.
Limbo et al. (2007). Ctp1 is a cell-cycle-regulated protein that functions with Mre11 complex to control doublestrand break repair by homologous recombination. Mol Cell 28, 134146.
Liu, F., and Lee, W. H. (2006). CtIP activates its own and cyclin D1 promoters via the E2F/RB pathway during G1/S progression. Mol Cell Biol 26, 3124-3134.
Luo et al. (1999). Disruption of mRad50 causes embryonic stem cell lethality, abnormal embryonic development, and sensitivity to ionizing radiation. Proc Natl Acad Sci USA 96, 7376-7381.
Ma et al. (2002). Hairpin opening and overhang processing by an Artemis/DNA-dependent protein kinase complex in nonhomologous end joining and V(D)J recombination. Cell 108, 781-794.
Milligan et al. (2000). H19 gene expression is up-regulated exclusively by stabilization of the RNA during muscle cell differentiation. Oncogene, 19, 5810-5816.
Mirzoeva, O. K., and Petrini, J. H. (2003). DNA replication-dependent nuclear dynamics of the Mre11 complex. Mol Cancer Res 1, 207-218.
Mohan et al (2007). Effect of doxycycline-regulated protein disulfide isomerase expression on the specific productivity of recombinant CHO cells: thrombopoietin and antibody. Biotechnol. Bioeng. 98(3), 611-615.
Monferran et al. (2004). The membrane form of the DNA repair protein Ku interacts at the cell surface with metalloproteinase 9. Embo J 23, 3758-3768.
Moore, J. K., and Haber, J. E. (1996). Cell cycle and genetic requirements of two pathways of nonhomologous end-joining repair of double-strand breaks in Saccharomyces cerevisiae. Mol Cell Biol 16, 2164-2173.
Moshous et al. (2001). Artemis, a novel DNA doublestrand break repair/V(D)J recombination protein, is mutated in human severe combined immune deficiency. Cell 105, 177-186.
Muller et al. (2005). The double life of the Ku protein: facing the DNA breaks and the extracellular environment. Cell Cycle 4, 438-441.
Newton, Harris et al. (1998) A dominant interfering mutant of FADD/MORT1 enhances deletion of autoreactive thymocytes and inhibits proliferation of mature T lymphocytes. EMBO J. 17(3):706-18.
Nikjoo et al. (1998). Track structure in radiation biology: theory and applications. Int J Radiat Biol 73, 355-364.
O'Driscoll et al. (2001). DNA ligase IV mutations identified in patients exhibiting developmental delay and immunodeficiency. Mol Cell 8, 1175-1185.
O'Driscoll et al. (2003). A splicing mutation affecting expression of ataxia-telangiectasia and Rad3-related protein (ATR) results in Seckel syndrome. Nat Genet. 33, 497-501.
Pierce et al. (2001) Ku DNA end-binding protein modulates homologous repair of double-strand breaks in mammalian cells. Genes Dev, 15, 3237-3242.
Pool, M. R., J. Stumm, et al. (2002). “Distinct modes of signal recognition particle interaction with the ribosome.” Science 297(5585): 1345-8.
Rass et al. (2009). Role of Mre11 in chromosomal nonhomologous end joining in mammalian cells. Nat Struct Mol Biol 16, 819-824.
Recillas-Targa et al. (2002) Position-effect protection and enhancer blocking by the chicken beta-globin insulator are separable activities. Proc Natl Acad Sci USA, 99, 6883-6888.
Robertson et al. (1995) Position-dependent variegation of globin transgene expression in mice. Proc Natl Acad Sci USA, 92, 5371-5375.
Sartori et al. (2007). Human CtIP promotes DNA end resection. Nature 450, 509-514.
Schaeper et al. (1998). Interaction between a cellular protein that binds to the C-terminal region of adenovirus E1A (CtBP) and a novel cellular protein is disrupted by E1A through a conserved PLDLS motif. J Biol Chem 273, 8549-8552.
Shen et al. (1996). Specific interactions between the human RAD51 and RAD52 proteins. J Biol Chem 271, 148-152.
Song, B., and Sung, P. (2000). Functional interactions among yeast Rad51 recombinase, Rad52 mediator, and replication protein A in DNA strand exchange. J Biol Chem 275, 15895-15904.
Stewart et al. (1999). The DNA double-strand break repair gene hMRE11 is mutated in individuals with an ataxia-telangiectasia-like disorder. Cell 99, 577-587.
Stiff et al. (2004). ATM and DNA-PK function redundantly to phosphorylate H2AX after exposure to ionizing radiation. Cancer Res 64, 2390-2396.
Sugiyama et al. (1997). A single-stranded DNA binding protein is needed for efficient presynaptic complex formation by the Saccharomyces cerevisiae Rad51 protein. J Biol Chem 272, 7940-7945.
Sun et al. (1991). Extensive 3′-overhanging, single-stranded DNA associated with the meiosis-specific double-strand breaks at the ARG4 recombination initiation site. Cell 64, 1155-1161.
Sung, P., and Klein, H. (2006). Mechanism of homologous recombination: mediators and helicases take on regulatory functions. Nat Rev Mol Cell Biol 7, 739-750.
Symington, L. S. (2002). Role of RAD52 epistasis group genes in homologous recombination and double-strand break repair. Microbiol. Mol Biol Rev 66, 630-670.
Szostak et al. (1983) The double-strand-break repair model for recombination. Cell, 33, 25-35.
Takata et al. (1998) Homologous recombination and non-homologous end-joining pathways of DNA double-strand break repair have overlapping roles in the maintenance of chromosomal integrity in vertebrate cells. Embo J, 17, 5497-5508.
Takeda et al. (2007). Ctp1/CtIP and the MRN complex collaborate in the initial steps of homologous recombination. Mol Cell 28, 351-352.
Taniguchi, T., and D'Andrea, A. D. (2006). Molecular pathogenesis of Fanconi anemia: recent progress. Blood 107, 4223-4233.
Taylor et al. (2004). Ataxia-telangiectasia-like disorder (ATLD)—its clinical presentation and molecular basis. DNA Repair (Amst) 3, 1219-1225.
Tourneur and Chiocchia (2010). FADD: a regulator of life and death. Trends Immunol, 31, 260-269.
Urlaub et al. (1983) Deletion of the diploid dihydrofolate reductase locus from cultured mammalian cells. Cell, 33, 405-412.
Van den Bosch et al. (2003). The MRN complex: coordinating and mediating the response to broken chromosomes. EMBO Rep 4, 844-849.
Wakimoto, B. T. (1998) Beyond the nucleosome: epigenetic aspects of position-effect variegation in Drosophila. Cell, 93, 321-324.
Walter, P. and G. Blobel (1981). “Translocation of proteins across the endoplasmic reticulum III. Signal recognition protein (SRP) causes signal sequence-dependent and site-specific arrest of chain elongation that is released by microsomal membranes.” J Cell Biol 91(2 Pt 1): 557-61.
Wang et al. (2005a). DNA ligase III as a candidate component of backup pathways of nonhomologous end joining. Cancer Res 65, 4020-4030.
Wang et al. (2005b). Artemis deficiency confers a DNA double-strand break repair defect and Artemis phosphorylation status is altered by DNA damage and cell cycle progression. DNA Repair (Amst) 4, 556-570.
Waterman et al. (1998). ATM dependent activation of p53 involves dephosphorylation and association with 14-3-3 proteins. Nat Genet. 19, 175-178.
West, S. C. (2003) Molecular views of recombination proteins and their control. Nat Rev Mol Cell Biol, 4, 435-445.
White, C. I., and Haber, J. E. (1990). Intermediates of recombination during mating type switching in Saccharomyces cerevisiae. Embo J 9, 663-673
Williams et al. (2007). Mre11-Rad50-Nbs1 is a keystone complex connecting DNA repair machinery, double-strand break signaling, and the chromatin template. Biochem Cell Biol 85, 509-520.
Wilson et al. (1999). The role of Schizosaccharomyces pombe Rad32, the Mre11 homologue, and other DNA damage response proteins in non-homologous end joining and telomere length maintenance. Nucleic Acids Res 27, 2655-2661.
Wold, M. S. (1997). Replication protein A: a heterotrimeric, single-stranded DNA-binding protein required for eukaryotic DNA metabolism. Annu Rev Biochem 66, 61-92.
Wong et al. (1998). Characterization of a carboxy-terminal BRCA1 interacting protein. Oncogene 17, 2279-2285.
Wong, E. A. and Capecchi, M. R. (1986) Analysis of homologous recombination in cultured mammalian cells in transient expression and stable transformation assays. Somat Cell Mol Genet, 12, 63-72.
Wu, G., and Lee, W. H. (2006). CtIP, a multivalent adaptor connecting transcriptional regulation, checkpoint control and tumor suppression. Cell Cycle 5, 1592-1596.
Xiao, Y., and Weaver, D. T. (1997). Conditional gene targeted deletion by Cre recombinase demonstrates the requirement for the double-strand break repair Mre11 protein in murine embryonic stem cells. Nucleic Acids Res 25, 2985-2991.
Yan et al. (2007). IgH class switching and translocations use a robust non-classical end-joining pathway. Nature 449, 478-482.
Yaneva et al. (1997). Interaction of DNA-dependent protein kinase with DNA and with Ku: biochemical and atomic-force microscopy studies. Embo J 16, 5098-5112
Yang et al. (1996). p300/CBP-associated factor that competes with the adenoviral oncoprotein EIA. Nature 382, 319-324.
Yasui et al. (2002) SATB1 targets chromatin remodelling to regulate genes over long distances. Nature, 419, 641-645.
You et al. (2009). CtIP links DNA double-strand break sensing to resection. Mol Cell 36, 954-969.
Yun, M. H., and Hiom, K. (2009). CtIP-BRCA1 modulates the choice of DNA double-strandbreak repair pathway throughout the cell cycle. Nature 459, 460-463.
Zahn-Zabal et al. (2001). Development of stable cell lines for production or regulated expression using matrix attachment regions. J Biotechnol 87, 29-42.
Zhu et al. (2001). Targeted disruption of the Nijmegen breakage syndrome gene NBS1 leads to early embryonic lethality in mice. Curr Biol 11, 105-109.

1. A method for transgene expression comprising:
(a) providing an eukaryotic, preferably a mammalian, host cell, wherein said host cell has been modified or treated to increase homologous recombination (HR), decrease non homologous end joining (NHEJ) and/or to enhance HR/NHEJ ratio in said cell, and
(b) transfecting said cell,
with at least one vector comprising said transgene, and
with, optionally, a matrix attachment region (MAR) element,
wherein said MAR element is provided to said transgene in cis or trans.

2. The method of claim 1, wherein the transfection in (b) is a subsequent transfection and is preceded by an initial transfection with nucleic acid such as a vector or nucleic acid fragments.

3. The method of claim 2, wherein a cell cycle of a cell population of said cell is synchronized.

4. The method of claim 2, wherein the said initial and subsequent transfection takes place at a time when a majority of the cells of the population are at the G1 phase of the cell cycle and, optionally more than 30%, more than 31%, 32%, 33%, 34%, 35%, 36%, 36%, 38%, 39%, 40%, 41%, 42%, 43%, 44% or 45% of the cells of the cell population are in the G1 phase.

5. (canceled)

6. The method of claim 3, wherein the cell cycle is synchronized by subjecting the cell population to a chemical or temperature treatment.

7. The method of claim 2, wherein an HR enzyme, an HR activator and/or a NHEJ suppressor is administered to said cell prior to said initial transfection.

8. The method of claim 1, wherein said cell is a recombinant eukaryotic host cell and comprises a transgenic sequence encoding an HR enzyme, an HR activator and/or a NHEJ suppressor and/or wherein said cell is mutated in a NHEJ or a HR gene.

9. The method of claim 1, wherein said cell is a recombinant eukaryotic host cell and the genome of said cell is mutated to inactivate NHEJ, to increase expression or activity of at least one HR enzyme, at least one HR activator and/or at least one NHEJ suppressor.

10. (canceled)

11. (canceled)

12. The method of claim 1, wherein the HR/NHEJ ratio of the cell is up to 2, 3, 4, 5, 6, 7, 8, 9, 10, 15, 20, 25, 30 times higher than a ratio found in the cell not comprising said transgenic sequence and not being mutated, respectively or wherein the NHEJ activity equals 0.

13. The method of claim 1, wherein an integrated copy number of said transgene integrated into the genome of said cell following said at least one subsequent transfection is more than twice that of a reference value representing the integrated copy number obtained by directly transfection the cell with the vector of (b).

14. The method of claim 2, wherein said at least one initial transfection is a single transfection and optionally the nucleic acid of the initial transfection is a vector comprising a MAR element and said transgene and, wherein, following the initial transfection, the expression of said transgene reaches an initial level and, wherein the expression of the transgene following the subsequent transfection, such as a single transfection, reaches a subsequent level that is more than additive, preferably, after a single subsequent transfection, more than twice, three or four times that of said initial level.

15. (canceled)

16. The method of claim 14, wherein the nucleic acid in (a) is a vector comprising a MAR element and said transgene and, wherein, after the initial transfection, the transgene copy number integrated into the genome of the cell equals (n) and, wherein following the at least one subsequent transfection, the transgene copy number integrated into the genome is more than 2(n), 3(n) or 4(n).

17. (canceled)

18. The method of claim 14, wherein the transgene is integrated into the genome of said cell as a concatemeric structure at a single locus.

19. The method of claim 1, wherein the MAR element in (b) ameliorates expression, substantially or fully prevents inhibitory effects from co-integration of multiple copies of the vector comprising the transgene.

20. The method of claim 2, wherein more than 50%, 60%, 70%, 80% of the vectors of the at least one subsequent transfection are transported into the nucleus.

21. The method claim 1, wherein, following the initial transfection, an initial level of transgene expression product and an initial transgene copy number is reached, and wherein, following said at least one subsequent transfection, the level of transgene expression product increases to a subsequent level and the initial transgene copy number increases to a subsequent transgene copy number, wherein the increase between the first and second level of transgene expression product exceeds the increase between the initial transgene copy number and the subsequent transgene copy number by 20%, 30%, 40%, 50% or 60%.

22. (canceled)

23. (canceled)

24. (canceled)

25. (canceled)

26. The method of claim 1, wherein said at least one MAR element in (b) is provided in cis as part of the vector in (b) and the transgene is flanked by said at least two MAR elements.

27. (canceled)

28. The method of claim 1, wherein the MAR sequence has at least 90% sequence identity with: SEQ ID NOs: 1-3 or is a variant thereof.

29. A recombinant eukaryotic, preferably mammalian, host cell, comprising
(a) transgenic sequence expressing a NHEJ suppressor,
(b) a transgenic sequence expressing one or more HR enzymes or HR activators,
(c) a mutation inactivating or downregulating a NHEJ gene, and/or
(d) a mutation enhancing expression or activity of an HR enzyme, an HR activator or a NHEJ suppressor,
wherein the recombinant eukaryotic host cell
has an HR/NHEJ ratio more than 2, 3, 4, 5, 6, 7, 8, 9, 10, 15, 20, 25, 30 times higher than a ratio found in the cell not comprising said transgenic sequence of (a) and/or (b), and
comprises, optionally, a matrix attachment region (MAR) element.

30. A recombinant eukaryotic, preferably mammalian, host cell, comprising
(a) a transgenic sequence expressing a NHEJ suppressor,
(b) a transgenic sequence expressing one or more HR enzymes or HR activators,
(c) a mutation inactivating or downregulating a NHEJ gene, and/or
(d) a mutation enhancing expression or activity of a HR enzyme, a HR activator or a NHEJ suppressor, and
a transgene integrated into the genome of said cell, and
optionally, a MAR element, wherein said MAR element is provided in cis or trans to said transgene.

31. The cell of claim 29, wherein the one or more HR enzymes are Rad 51, Rad 52, RecA, Rad 54, RuvC or BRCA2 and/or the HR activator is RS-1 and/or the NHEJ suppressor is NU7026 and/or wortmannin.

32. (canceled)

33. The cell of claim 29, wherein said mutation in (c) or (d) is a mutation in a xrcc4 gene, RAD51 strand transferase gene, a DNA-dependent protein kinase gene, the Rad 52 gene, the RecA gene, the Rad 54 gene, the RuvC gene and/or the BRCA2 gene.

34. The cell of claim 29, wherein the transgene is integrated into one locus of the genome of the cell and forms a concatemeric structure, optionally comprising at least 200, 300, 400, 500 or 600 copies of the transgene.

35. (canceled)

36. A recombinant eukaryotic, preferably mammalian host cell, comprising integrated into a single locus of the genome a concatemeric structure of a transgene functionally linked to a promoter, wherein the concatemeric structure comprises at least 300, 400, 500 or 600 copies of the transgene and at least one MAR element, wherein said MAR element is provided in cis or trans to said transgene, wherein said cell is preferably part of a cell population that has been synchronized.

37. The cell of claim 36, wherein the at least one MAR is provided in cis and the majority of said transgenes are provided with a MAR for each of said transgenes or wherein the transgene is flanked by at least two of said MAR elements.

38. (canceled)

39. The cell of claim 29, wherein the at least one MAR element has at least 90% sequence identity with SEQ ID NOs: 1-3 or is a variant of SEQ ID NOs: 1-3.

40. The cell of claim 29, wherein the MAR element is located upstream of a promoter/enhancer sequence of said transgene.

41. The cell of claim 29, wherein the cell is a CHO cell, a HEK 293 cell, a stem cell or a progenitor cell.

42. (canceled)

43. A kit comprising
(a) in a first container, a vector comprising optionally a MAR element and restriction sites for integration of a transgene into said vector,
(b) in a second container, a recombinant eukaryotic host cell of claim 29,
(c) instructions how to use said vector in transfecting said cell for transgene expression and optionally
(d) a synchronizing agent or instructions on how to synchronize a cell population comprising said cell(s).

44. (canceled)

45. The kit of claim 43, wherein the vector is used to transfect the cell with said vector at least twice when the majority of the cells of said cell population is at the G1 phase.

46. A non-primate recombinant eukaryotic host cell, such as a rodent cell, preferably a CHO cell, comprising
a transgenic sequence encoding at least one primate protein or a primate RNA involved in translocation across the endoplasmic reticulum (ER) membrane and/or secretion across the cytoplasmic membrane, such as a protein or a RNA of a signal recognition particle (SRP) or a protein of a secretory complex (translocon) or a subunit thereof.

47. The cell of claim 46, wherein said cell further comprises a transgene such as a immunoglobulin, a subunit or fragment thereof or a fusion protein functionally attached to a signal peptide coding sequence, wherein the signal peptide coding sequence preferably has at least 90% sequence identity with SEQ ID NOs: 4-11 or is a variant of any one of said sequences, and wherein said transgene is present in the cell in multiple copies, preferably in form of a concatemeric structure such as at least 200, 300, 400, 500 or 600 copies of the transgene and, optionally further comprising an epigenetic regulator element, such as an MAR element, located in cis or trans to said transgene.

48. (canceled)

49. (canceled)

50. (canceled)

51. The cell of claim 46, wherein said protein or RNA involved in translocation across the ER membrane and/or secretion across the cytoplasmic membrane is a protein or RNA of the SRP, in particular SRP9, SRP14, SRP19, SRP54, SRP68, SRP72 and/or 7SRNA.

52. The cell of claim 51, wherein the protein of the SRP is human SRP14 or SRP 54, preferably combined with one or more other of said proteins, such as human SR and/or human Translocon proteins, or RNA involved in in translocation across the ER membrane and/or secretion across the cytoplasmic membrane.

53. (canceled)

54. (canceled)

55. (canceled)

56. The cell of claim 46, wherein said protein or RNA involved in in translocation across the ER membrane and/or secretion across the cytoplasmic membrane is the one of the proteins of the translocon, in particular Sec61αβγ, Sec62, Sec63 and/or a subunit thereof or is a combination of SRP9, SRP14 and a Translocon protein.

57. (canceled)

58. (canceled)

59. (canceled)

60. (canceled)

61. (canceled)

62. A kit comprising
(a) in one container, non-primate recombinant host cell comprising, as part of the genome of the cell, a transgenic sequence encoding at least one protein or a RNA involved in in translocation across the ER membrane and/or secretion across the cytoplasmic membrane, such as a protein or a RNA of a signal recognition particle (SRP) or a protein of a secretory complex (translocon) or a subunit thereof,
(b) in a separate container, at least one vector comprising restriction sites for integration of a transgene into said vector and optionally a MAR element, and
(c) instructions for expressing and secreting a transgene expression product of said transgene using said cell.

63. A method for protein secretion of a transgene comprising:
providing a non-primate eukaryotic host cell comprising
(a) a transgenic sequence encoding at least one primate protein or a primate RNA involved in in translocation across the ER membrane and/or secretion across the cytoplasmic membrane, such as a protein or a RNA of a signal recognition particle (SRP) or a protein of a secretory complex (translocon) or a subunit thereof, said transgenic sequence preferably having at least 90% sequence identity with a sequence selected from the group of SEQ ID NOs: 4-11 or is a variant of any one of said sequences, and
(b) a transgene functionally attached to a signal peptide coding sequence and
(c) secreting said transgene.

64. The method for protein secretion of claim 63, wherein said transgenic sequence increases a total amount of protein or RNA involved in in translocation across the ER membrane and/or secretion across the cytoplasmic membrane present in said cell by more than 10%, 20%, 30%, 40% 50%, 60%, 70%, 80%, 90% or 100% above a level found in the cell prior to expressing said transgenic sequence.

65. (canceled)

66. (canceled)

67. (canceled)

68. (canceled)

69. (canceled)

70. A method for identifying a protein secretion and/or translocation increasing activity of a transgenic sequence comprising:
monitoring a first mammalian cell comprising a transgene encoding a recombinant protein, wherein said recombinant protein is secreted by said cell at a first level,
monitoring a second mammalian cell comprising said transgene encoding said recombinant protein, wherein the recombinant protein is secreted by said cell at a second level,
wherein said second level exceeds said first level,
introducing into said first mammalian cell the transgenic sequence encoding at least one protein or a RNA involved in translocation across the ER membrane and/or secretion across the cytoplasmic membrane, and
determining changes in the secretion level of said recombinant protein in said first cell,
wherein an increase beyond the first level identifies the protein secretion increasing activity of said transgenic sequence.
